SEQUENCE LISTING (88 sequences)

SEQ ID NO: 1 - DNA, 56 bp, Artificial Sequence
  Description of Artificial Sequence Synthetic oligonucleotide
gtaaggtaagaattgaatttctcagttgaaggatgcttacactcttgtccatctag

SEQ ID NO: 2 - DNA, 49 bp, Artificial Sequence
  Description of Artificial Sequence Synthetic oligonucleotide
gtgtgtatgctcaggggctgggaaaggaggggagggagctccggctcag

SEQ ID NO: 3 - DNA, 266 bp, Artificial Sequence
  Description of Artificial Sequence Synthetic polynucleotide
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtaccgtaaggtaagaattgaatttctcagttgaaggatgcttacactcttgtccatctag

SEQ ID NO: 4 - DNA, 259 bp, Artificial Sequence
  Description of Artificial Sequence Synthetic polynucleotide
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtaccgtgtgtatgctcaggggctgggaaaggaggggagggagctccggctcag

SEQ ID NO: 5 - DNA, 117 bp, Artificial Sequence
  Description of Artificial Sequence Synthetic polynucleotide
gtgatgacgtgtcccataaggcccctcggtctaaggcttccctatttcctggttcgccggcggccattttgggtggaagcgatagctgagtggcggcggctgctgattgtgttctag

SEQ ID NO: 6 - DNA, 117 bp, Artificial Sequence
  Description of Artificial Sequence Synthetic polynucleotide
gtgatgacgtgtcccatacttccgggtcaggtgggccggctgtcttgaccttctttgcggctcggccattttgtcccagtcagtccggaggctgcggctgcagaagtaccgcctgcg

SEQ ID NO: 7 - DNA, 117 bp, Artificial Sequence
  Description of Artificial Sequence Synthetic polynucleotide
gtgatgacgtgtcccatattttcatctcgcgagacttgtgagcggccatcttggtcctgccctgacagattctcctatcggggtcacagggacgctaagattgctacctggactttc

SEQ ID NO: 8 - DNA, 117 bp, Artificial Sequence
  Description of Artificial Sequence Synthetic polynucleotide
gtgatgacgtgtcccatggcctcattggatgagaggtcccacctcacggcccgaggcggggcttctttgcgcttaaaagccgagccgggccaatgttcaaatgcgcagctcttagtc

SEQ ID NO: 9 - DNA, 117 bp, Artificial Sequence
  Description of Artificial Sequence Synthetic polynucleotide
gtgatgacgtgtcccatcccccctccaccccctagcccgcggagcacgctgggatttggcgcccccctcctcggtgcaacctatataaggctcacagtctgcgctcctggtacacgc

SEQ ID NO: 10 - DNA, 100 bp, Artificial Sequence
  Description of Artificial Sequence Synthetic polynucleotide
cccccctccaccccctagcccgcggagcacgctgggatttggcgcccccctcctcggtgcaacctatataaggctcacagtctgcgctcctggtacacgc

SEQ ID NO: 11 - DNA, 100 bp, Artificial Sequence
  Description of Artificial Sequence Synthetic polynucleotide
ggcctcattggatgagaggtcccacctcacggcccgaggcggggcttctttgcgcttaaaagccgagccgggccaatgttcaaatgcgcagctcttagtc

SEQ ID NO: 12 - DNA, 100 bp, Artificial Sequence
  Description of Artificial Sequence Synthetic polynucleotide
gggtggggcccgcgcgtataaagggggcgcaggcgggctgggcgttccacaggccaagtgcgctgtgctcgaggggtgccggccaggcctgagcgagcga

SEQ ID NO: 13 - DNA, 100 bp, Artificial Sequence
  Description of Artificial Sequence Synthetic polynucleotide
ggtgcgatattcggattggctggagtcggccatcacgctccagctacgccacttccttttcgtggcactataaagggtgctgcacggcgcttgcatctct

SEQ ID NO: 14 - DNA, 100 bp, Artificial Sequence
  Description of Artificial Sequence Synthetic polynucleotide
acttccgggtcaggtgggccggctgtcttgaccttctttgcggctcggccattttgtcccagtcagtccggaggctgcggctgcagaagtaccgcctgcg

SEQ ID NO: 15 - DNA, 100 bp, Artificial Sequence
  Description of Artificial Sequence Synthetic polynucleotide
gctgagcgcgcgcgatggggcgggaggtttggggtcaaggagcaaactctgcacaagatggcggcggtagcggcagtggcggcgcgtaggaggcggtgag

SEQ ID NO: 16 - DNA, 100 bp, Artificial Sequence
  Description of Artificial Sequence Synthetic polynucleotide
attttcatctcgcgagacttgtgagcggccatcttggtcctgccctgacagattctcctatcggggtcacagggacgctaagattgctacctggactttc

SEQ ID NO: 17 - DNA, 100 bp, Artificial Sequence
  Description of Artificial Sequence Synthetic polynucleotide
tgggacccccggaaggcggaagttctagggcggaagtggccgagaggagaggagaatggcggcggaaggctggatttggcgttggggctggggccggcgg

SEQ ID NO: 18 - DNA, 100 bp, Artificial Sequence
  Description of Artificial Sequence Synthetic polynucleotide
aaggcccctcggtctaaggcttccctatttcctggttcgccggcggccattttgggtggaagcgatagctgagtggcggcggctgctgattgtgttctag

SEQ ID NO: 19 - DNA, 100 bp, Artificial Sequence
  Description of Artificial Sequence Synthetic polynucleotide
agtgacccggaagtagaagtggcccttgcaggcaagagtgctggagggcggcagcggcgaccggagcggtaggagcagcaatttatccgtgtgcagcccc

SEQ ID NO: 20 - DNA, 100 bp, Artificial Sequence
  Description of Artificial Sequence Synthetic polynucleotide
gggaggggcgcgctggggagcttcggcgcatgcgcgctgaggcctgcctgaccgaccttcagcagggctgtggctaccatgttctctcgcgcgggtgtcg

SEQ ID NO: 21 - DNA, 100 bp, Artificial Sequence
  Description of Artificial Sequence Synthetic polynucleotide
actgcgcacgcgcgcggtcgcaccgattcacgcccccttccggcgcctagagcaccgctgccgccatgttgagggggggaccgcgaccagctgggcccct

SEQ ID NO: 22 - DNA, 100 bp, Artificial Sequence
  Description of Artificial Sequence Synthetic polynucleotide
ccctcgaggggcggagcaaaaagtgaggcagcaacgcctccttatcctcgctcccgctttcagttctcaataaggtccgatgttcgtgtataaatgctcg

SEQ ID NO: 23 - DNA, 100 bp, Artificial Sequence
  Description of Artificial Sequence Synthetic polynucleotide
cttggtgaccaaatttgaaaaaaaaaaaaaaccgcgccaactcatgttgttttcaatcaggtccgccaagtttgtatttaaggaactgtttcagttcata

SEQ ID NO: 24 - DNA, 100 bp, Artificial Sequence
  Description of Artificial Sequence Synthetic polynucleotide
ggctgagctatcctattggctatcgggacaaaatttgcttgagccaatcaaagtgctccgtggacaatcgccgttctgtctataaaaaggtgaagcagcg

SEQ ID NO: 25 - DNA, 100 bp, Artificial Sequence
  Description of Artificial Sequence Synthetic polynucleotide
ggaagtgccagaccggaggtgcgtcattcaccggcgacgccgatacggttcctccaccgaggcccatgcgaagctttccactatggcttccagcactgtc

SEQ ID NO: 26 - DNA, 100 bp, Artificial Sequence; identical to SEQ ID NO: 22
  Description of Artificial Sequence Synthetic polynucleotide

SEQ ID NO: 27 - DNA, 100 bp, Artificial Sequence; identical to SEQ ID NO: 23
  Description of Artificial Sequence Synthetic polynucleotide

SEQ ID NO: 28 - DNA, 100 bp, Artificial Sequence; identical to SEQ ID NO: 24
  Description of Artificial Sequence Synthetic polynucleotide

SEQ ID NO: 29 - DNA, 100 bp, Artificial Sequence; identical to SEQ ID NO: 25
  Description of Artificial Sequence Synthetic polynucleotide

SEQ ID NO: 30 - DNA, 204 bp, Artificial Sequence
  Description of Artificial Sequence Synthetic polynucleotide
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 31 - DNA, 278 bp, Artificial Sequence
  Description of Artificial Sequence Synthetic polynucleotide
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 32 - DNA, 305 bp, Artificial Sequence
  Description of Artificial Sequence Synthetic polynucleotide
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 33 - DNA, 80 bp, Artificial Sequence
  Description of Artificial Sequence Synthetic oligonucleotide
gtacttatataagggggtgggggcgcgttcgtcctcagtcgcgatcgaacactcgagccgagcagacgtgcctacggacc

SEQ ID NO: 34 - DNA, 279 bp, Artificial Sequence
  Description of Artificial Sequence Synthetic polynucleotide
ggggaggctgctggtgaatattaaccaaggtcaccccagttatcggaggagcaaacaggggctaagtccacgctagcgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtc

SEQ ID NO: 35 - DNA, 262 bp, Artificial Sequence
  Description of Artificial Sequence Synthetic polynucleotide
gtttgctgcttgcaatgtttgcccattttagggtggacacaggacgctgtggtttctgagccagggctagcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgccac

SEQ ID NO: 36 - DNA, 203 bp, Artificial Sequence
  Description of Artificial Sequence Synthetic polynucleotide
tgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 37 - DNA, 251 bp, Artificial Sequence
  Description of Artificial Sequence Synthetic polynucleotide
gtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 38 - DNA, 304 bp, Artificial Sequence
  Description of Artificial Sequence Synthetic polynucleotide
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 39 - DNA, 1643 bp, Artificial Sequence
  Description of Artificial Sequence Synthetic polynucleotide
gttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaatt

SEQ ID NO: 40 - DNA, 231 bp, Artificial Sequence
  Description of Artificial Sequence Synthetic polynucleotide
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagg

SEQ ID NO: 41 - DNA, 681 bp, Homo sapiens
ggaggaagccatcaactaaactacaatgactgtaagatacaaaattgggaatggtaacatattttgaagttctgttgacataaagaatcatgatattaatgcccatggaaatgaaagggcgatcaacactatggtttgaaaagggggaaattgtagagcacagatgtgttcgtgtggcagtgtgctgtctctagcaatactcagagaagagagagaacaatgaaattctgattggccccagtgtgagcccagatgaggttcagctgccaactttctctttcacatcttatgaaagtcatttaagcacaactaactttttttttttttttttttttttgagacagagtcttgctctgttgcccaggacagagtgcagtagtgactcaatctcggctcactgcagcctccacctcctaggctcaaacggtcctcctgcatcagcctcccaagtagctggaattacaggagtggcccaccatgcccagctaatttttgtatttttaatagatacgggggtttcaccatatcacccaggctggtctcgaactcctggcctcaagtgatccacctgcctcggcctcccaaagtgctgggattataggcgtcagccactatgcccaacccgaccaaccttttttaaaataaatatttaaaaaattggtatttcacatatatactagt

SEQ ID NO: 42 - DNA, 502 bp, Mus musculus
agtttggacaagaactatagttctagctttctctgggtctccaccttgcagagaatgcagctttcattatctcatgagccaaactctcatcatctctttccatatatctgtcggtgctcttccatgagtactctaacacacacagaaggagcacttacacaggctgttgtttttctcttattatcatagctgttgttcagacatgtgcattctgttcttgttgcttcaatgctaaaggagtctcaggatatgagaactgtaccagccgaggcatcaggaaacatgggtggaaattcccacagtactatttgttcactgtgtgaccttgggccagtcacatccctttcctgaggcttcgattccccaagctataaaagaagcatctcttaacctttttttaggtcatgagtcaggcccagcacactctcagggagactcatgagagtacagatcatttcccatagaaaaaccatagttttatatccagaggcttttctgtaag

SEQ ID NO: 43 - DNA, 823 bp, Mus musculus
ggttccagttcagaggcagagcatttggggttcccagtcaggagctttcctctctccgctccttagtttcctctctttaaaaaaaaatgggtgatagtatagaaaggaagctctgggctcggggaccagggccctgggatccccgctcccagccactcgctcctgacccttccagggacaagctcccccccaccccgtcctttccaggctgccactagaagagatggggacgcgtggtcagccgcttctgtcgccccccagggaacggtctcacgctggagggggcagtgccctcggaacaggacagtcagcccaagccagccaagcgcgcgcggacgtccttcaccgcagagcaattgcaggtaccccgggcaagccccgaagcgtgtgggcggggcttcggagtgggcgtggttgttcgggacttgtgactccgccccttgtgcggggacccgcgtgaggccgctccaaggatgaagctgcctggggcgtggcctcggaccctgagcctctgattgggcggaggtctcagggcccttctgcgccccacaggttatgcaggcgcagttcgcgcaggacaacaacccggacgcgcagacgctgcagaagctggcggacatgacgggcctcagtcgcagggtcatccaggtggggctccggggtctcggccttcaggtctagggtgaaccttagggaagcgctgaagctcgtagtggtacggatggtcgcgcgtgcacgtggccgcccctctccagtgtggcctaaggaccccagtcggcacgggttgacccttttccttgattactgagagtgcagaggctgt

SEQ ID NO: 44 - DNA, 633 bp, Mus musculus
tggtgggaagacatgtccagggaagaaatggcctccagaggcctgaggtggggaaatgctggaggtggagagaggaacaactgactgaaaatgagcttccactgtggcttagtagcctataccaagtctagagtatagggtaggagaagattaggaaagcgatgggtctgagaatgatgtggcctgttgacttttgtaaacccaaagcaccttggactaaaccctatgaacagtgtggtgccaccaaagactataatgagctcagggaacagaattctgtgtgcatggtgattttttttttttttttctgctaactgcagtctgggtgatgcattgacaaaccaatcctggaaagtaagaggcaagggcagctgggacggtgagaggagcctgatgggaaccaggccaagcagggcagcagaggcgatgaagaggatgtggtgcatccagagactcacttcattagctggaggcactgctggatagggtctgaaggttctggtatctgagttggcgggctgggtgagtggtggctctgcttcctgaacagtgtgtgcaagaggaaacagggttaagggctaggacagtcacaggtgagtcagcctcacaagagcaaccttcccctagtgcaga

SEQ ID NO: 45 - DNA, 670 bp, Mus musculus
ggaggtctccttttgccccggttccaacaagagaatgcaaggctgtatctcaatttccttgagcctctctgtattatagaagaaaagtagggaagccatacgccccttctgagcttcagtgtctctctgtctctgcaaatgaggctggggaggctgggggcgggcgtgaaagaggcccgcgccaagccgacccccacctctgccccctccccaggtcaacaacctcatctggcacgtgcggtgcctcgagtgctccgtgtgtcgcacatcgctgaggcagcagaatagctgctacatcaagaacaaggagatctactgcaagatggactacttcaggtaggcagcggccatcccgccagcaagcgctggagcatgaacgccttgcacacgcgtgcctaggccacttgtgtggcctgtgctctccaattcctgagccctgctgttcagagtgcacaacgcggctcagcgcactggcccggccctcctactcagcacgtcttacacagaagggagcgccagtctcagcctgagttctggcgggggatctgcctcgggttcctccgatctgacaggcgctggccacgggtctggttccatctctggtcttttctggccccgagcaccagtgtgttctgttgagctctgatgtccgaggctctggcccggatca

SEQ ID NO: 46 - DNA, 1043 bp, Mus musculus
ctctggctacctcttatcttgggcattcacgacaatttctaattgcaggtagtttgtgtgtgtgcgcgtgttttttttccccctcagaggcttggattgcaaaggaactaagcgattacttcaagagccacgggttaagtgcagggagagggggagagagagggaaaaaaacccaatccaaattcaaattgcttcattagagagacaccgcttttgtggggaagggctttaaatgcccactacaaagttaggactcattgttcagcgccggtttatataacaggcgaggggaggcgctgggctctgacagctccgagccagttcagcagccgccgtcgcctgcattccctccccctcccccaggtgatggcccagccagggtccggctgcaaagcgaccacccgctgtctcgaagggaccgctccgcctgccatggtgagtcctttcggtcctgctttcggccccgagtccccccaacagcacaggccagggcttctggctcagccttccggctaccaacctctacccctgcgctggaaaactgccgataggagccgcctctcgttgagccttggtttttctggcctggaatgtgagctttggctgcttcctgcacccaggatgcgctgtgttaaaagttgggggccgtcccttcttctccaataggtcctttcattcttgtactccagcctagggcgcgacatccctggcacatttcggtgtcagtcggtgcgcgaggaaaccagattcaactctgagtactcggctaagcgcttcgctgttcctctctcccatttcaggctcagtcagacgcagaggccttggcaggcgctctggacaaggacgaaggtagagcctccccatgtacgcccagcacaccgtctgtctgctcgccgccctctgctgcctcttccgtgccgtctgccggcaagaatatctgctccagttgcggtctggagatcctggaccggtatctgctcaaggtgagtcagggtaggtgtgcctgcttgcccacgggtgtggtttgcagccccaagagctgt

SEQ ID NO: 47 - DNA, 507 bp, Mus musculus
caagacttttaaaagtttagataaataaacaaacatttgacggctttccatcacatctagactataatccaaagatctatatggtcccaaacgacttacacttaactaccgtctcccatatggcttcttcccccatcagtcattgtcctcagccatagtggcctccctgttcctttgggtacaagggaacaactccctgagaggttccattagctgctgttgcctgagatgctcttgagcccacaccatctgctcatttctctcctcacgtgtcagtgattaagaggctgtccttggcctcccgtcaaaattacatccctgccgctttccacttcttgccttcttattttctaaatagaactaactcaccactacccaacattctatataattggatatctgtcctctgtttaaatataatgttgacttcaagaaagaacgttgtcactgccctgtcaccagacttttaaacagtgcctatcgtgtggcacatgctcagtgaaattg

SEQ ID NO: 48 - DNA, 502 bp, Mus musculus
tcaacagggggacacttgggaaagaaggatggggacagagccgagaggactgttacacattagagaaacatcagtgactgtgccagctttggggtagactgcacaaaagccctgaggcagcacaggcaggatccagtctgctggtcccaggaagctaaccgtctcagacagagcacaaagcaccgagacatgtgccacaaggcttgtgtagagaggtcagaggacagcgtacaggtcccagagatcaaactcaacctcaccaggcttggcagcaagcctttaccaacccacccccaccccacccaccctgcacgcgcccctctcccctccccatggtctcccatggctatctcacttggccctaaaatgtttaaggatgacactggctgctgagtggaaatgagacagcagaagtcaacagtagattttaggaaagccagagaaaaaggcttgtgctgtttttagaaagccaagggacaagctaagatagggcccaagtaat

SEQ ID NO: 49 - DNA, 788 bp, Mus musculus
aaatagaactgtgagatagggggagagggggcaggaaggacaagagacccctgtctcattgtgatccccacctgtctgctctgtgggagggtacccatgagggccagcccacagcccttaggtggacattgtctggtcctgtctcactgtccctcccagcagccccagaggccaggagacaggggtctcagtcctcactgagagatgtgtaaactgaggcccagtgaatgttgagggccagggcatgcccttggtgggatgtgacctgggtctccttcgcacgggcttcctccccgaagccgagctgagcatttggagtttgaaatgtttccgtacttagcaatctgctcctctattcccgggcggacttccgatagctccggccttatgctgcactagataagatggagcagggagaggacacggcactacttatgtaaccggcctcttgaaaaatggagcagcggtcagggcggaacaagacgtcctctctctacgcatccctctcctttccctgctaaggctgcagctggagtcagaggcagggctgttccaatctgtctttgatcagtaacgcagccagcctccagcctccgtcagcctcctcatggctgagacccggcctcagtttcccccacttacatcccgaggatcagagcctgtgaggatgaaatgggataaggtagctggaaccgtctggcagagagcgagtcctcaggactgttgatgcctgtggctgcctggcttgaccccaagtgaccccgcctcctcatcctgcagcaggagaa

SEQ ID NO: 50 - DNA, 502 bp, Mus musculus
tctatagaatgtgtccccagccttgttttccacacttgatacgcaaggaatgcataccacagagagggatgagggtagcatccagcctgcttcctgtgtgtcggggcgctacagccacatctccccagtccatctcagaccgtcacagagcttcgccgaatgtatagctttgttctctgtgcagacagggagacagagccttgggaagcataggtgcttgcttctttgcccactgagtcttagctggacttgcacaccacatgcctcacagccgggcgcacttgcatttgtcacccaggcccagtgatgatggctctgcttgctttgtgctttgtgccaactacagctccagcacctgtgccctgggttttcactcctttagttgaacacgtagttactggggttgtagggatggagcctttctgcttccttctggcaaagtccttagcggcctgctgcgggggtggggggtgttcaggggagtggtgatgaagtatgacag

SEQ ID NO: 51 - DNA, 533 bp, Mus musculus
tctccagttggagaaacagatgctgtaactggggccacagtataaagagagcccagacattgaactgtcaacacagaagcctggcacactggaactggcagtccagctgggaacaaggggtagaggctgaggccactaagtcaactgaggcaggagacataggagctaaagcagctgaagggtgcaggacagctggggggtctgaagtgggcctcatgcccagagctatgaagtcaggggctgtagcctaggagccttggaagccagctggcaagctgtggcccaaagacgctgactcaccaggagggggcagctggagccaggcactcctaaggtttccaggaagggcagccttccagggctcagctaggggagacagtgttgacagcaagttgtcaggcaacttgagctactgggcagctgggaagctgtcccttggtccccagtatcatcatcaccccagacgctgcccacctgcctcaggtcccacacagtgatcctcccatctttaacacaacacatgaccagagaga

SEQ ID NO: 52 - DNA, 524 bp, Mus musculus
gtcaccctccccccaaacaaccccttcttctctggttcgagaaattacaggcatgaaagatataaatcgggatgcttgacttgggaatataaatcactaaagcttgggggcaggggtgggcgacctttgtgaccgtccttgtgcgtgccagtaaatcctgtggtccaggggagaagaaaaggctgtgtggcttctgctcacaaagctgcagaaaccattctttaagcccaaaagcacttccagagagagcagagcatccccaggctgctggctcagcaagttcactgtgctcaatctcaggaagtgaggataagagcagtgcctggagagtgcctggtgctgagctgagggtttctgaacacattaaagcggggagcatggaccgggcctcaggaggggtgttgaacatccctaggcagaggagtctagcttcctgggaaaagatatcaggttaagcacacacatgtcctctggaataagataatctttctgatcacacactatacacacacaaaagcctgctc

SEQ ID NO: 53 - DNA, 509 bp, Mus musculus
gccctctaggccacctgaccaggtcccctcagtcccccccttcccacactcccacactcagcccccctcccccccccccgacccctgcaggattatcctgtctgtgttcctgactcagcctgggagccacctgggcagcaggggccaagggtgtcctagaagggacctggagtccacgctgggccaagcctgccctttctccctctgtcttccgtccctgcttgcggttctgctgaatgtggttatttctctggctccttttacagagaatgctgctgctaattttatgtggagctctgaggcagtgtaattggaagccagacaccctgtcagcagtgggctcccgtcctgagctgccatgcttcctgctctcctcccgtcccggctcctcatttcatgcagccacctgtcccagggagagaggagtcacccaggcccctcagtccgccccttaaataagaaagcctccgttgctcggcacacataccaagcagccgctggtgcaatct

SEQ ID NO: 54 - DNA, 398 bp, Mus musculus
gtgttcttcccttcccctttggacccccgagacaagccaataaaatactcggcagggtggcttctctcctttttttgccagtaataaacagactcagagcaagttaagggtctggtccaaggtcatggctgggatcagtgacagagcccagaagagaacctgagacttcttgctgagccaagctggagaggacagaaaggaatgcgtctactccatgcatgaccctctgccagctttgctccttcctaagggaccatgaacgatatgtgcacaccgctcatacgtatgtgcacacctgcaagaggaggcatcccatgtacacctatgagacgcacagagaaacatatatgtagccataggctagaaattctttctctttctaggtctgcccctctgca

SEQ ID NO: 55 - DNA, 810 bp, Mus musculus
ggaccactcagtgtacacggaatgtagaattgagtctgccattggtcttccctcaaagtcttggaggcttgggactgatattgggagcatctgggcagagaaggccacaaagacagggtggtttttctacactgggacatactcgtgagcatgcacagaggcgtgtccccaacttccctgtcacccctgtcctctgccggctagaggggatgcgggggtggacatatgctgctattgggcagatatcacatgttaagaggtgggggggggctcaagaggcggagggctaggagcatcccatggggagaggttctggttttcttgctgcctctagctgctataaatacgttagcacttgagcaactggaaagctctgagtaatttaggatgcacaaagctgtaatttaactccagcatctcagtgtgcgagagcattaaagatgtaattaagatgtttacacaaagagattggagtctgtgacacttggggtgcaaaaccccaggaagggacacaatgggtgaggtgaggatctgtgggaggcctggggacagtcacttggatcccagctatgagatggcaggccacccagctgtttctccttggaaatgttttggcctgggggttgggggtggggcatcacactttgatatggagatggggcaacaaagcctgcaatatctgggggtggagaggtcaagtggatggagtcttttgagatcatgtcaggaagagggctcgatcccccaaaatcatggtgacatatggtgtctcggggttcacaggagctatgtctaaaatacaaaagtaaa

SEQ ID NO: 56 - DNA, 202 bp, Mus musculus
tctgcagaagcctgccattccaccatttaaacctgtgactccaggccttaagcctgttgaaggtcgagtcccagaagggtcatatgtgcaactgcctagggagagttcccactcgcagggccaagaggagtcccccggtctgaggtgtgggggcggggacgtgcactgggcgctgggaccacggctggggctcaggactcgc

SEQ ID NO: 57 - DNA, 937 bp, Mus musculus
tgcctcagtttcttcgcctagaaagccgggtctaagggtacatgccctgattcttttctggggtgtctcgaattttaaacaacacatactgttctgggctgatgacaagaggaagtactggtcggtggctgatggacatccaccatggtggcaactggagggagggggaacggacgttgaaaccctgccctcctggaatctgtcgcatgcacgcacgttgacaatgcttggcactggggacaggctgggatggatggagcggagcgtgaggaggagtgggcatgcaggcccgagtgtctgttttgctgattgctccttttgctttcaaggagattaaactatttttagtccatgcctactgctggtgagacgctggaggaagcctttccatcgttgagattttctggaagctgccaagtgtggtcttcagctcaattctgggagcctcccagagtgggagggaggaacatttccatctgggggcttcggggacaggctaagatcttccctggggtccttgctgcgctggcctcctcaaaccacgctgcctcggcctgcataaagcagtaatctgatgtgcccgatgtttgtaacgctgtgtttaaaaaaagtaatttattttctaattattccttgtcttgcataaccatgcattgccaaagtgtcgctatttaaaatatttatctctccacgccgcaggagcagctctggagcgtggagggggaagaaataaaagtccgcgtgccagtcgcaggcatattactttgactcgtcctggtggctttgacgtctccctgtaaatacatttatttttcattaggacgtttctgagcttgtggcccccggagagcggagtgattacgctgttcatctgcaagcgatgcaatagaggggtactcgcagaatgacttccgcccagagcatcctgcgcctgtct

SEQ ID NO: 58 - DNA, 922 bp, Mus musculus
taaaataccttatttttttccagtctctaaactgctaatctcccaggctaagggattctgggacaaaggcaaggcctggaagtggaaatctgtaaaattagcttcagcggtattagtgtttgcagttgaagattgaaaaactgctttcccagggcctgattggaggctccactctcctccaggaagaggcaaggactctgggctggcactgaggacaaatcctgggaggctgctatggggcctgggagccaggctgccttgtgctagaggcctagagagtgtctgtgtcccaagtcccaagctacccccagcagctaacagcttttccagttctcaggcacagcaggtgccaagatcacgctctggagtccagctgggccccttcctcttctttttttttttttttttttaagacctcctggacactgttcctctccccccccccgtgaccccccccctcagttctcaaacacgtgagggttgggggagggttccacagccagagagaggggccagctctggtgcctgtgggtacgcccgcccgtatggcccatcaggcctcttgtgtgcttgattgcctctgattggctgcagctgaattcagcaaaagctattatttgcccttgatgagccaatcagatggcctcattggccattcagagcaggcaccggaacctgagggtggggtggggggtgggggatggagatgggactcagtgagggggtgggaagctctaaaacagatgcaggacctgagcctgtctgtgtccaccacgaccttcacacaggtcacacccccttcccctgacttgtcaccccaaaccagggcttgttgcccaaccccacctcacaattccctcactctgtaacacctttccatatacctctgcatgtctaaacccaagacttgctctatgaaatc

SEQ ID NO: 59 - DNA, 639 bp, Mus musculus
agaccctgcttagcacagctcttagcgggtcctttagggggtctcccagcgggcccagtgggaatgagataaggaaggacacagctgtccattctcccgtgcctgctaaggaggaaatggggccgccttacataattggggcaatttgttccactcttgtcctcctggtatcatggctatcaccccctccttgctcagggagtccttgattgagcgagaagctcaggcctccctctctccctcctgctgggggttgctgaacagagggtgtaggagccataggctctgtcactgctgagatctgccagatgtctaggccaggagaaaatggaaagggctaagtcacagcatatgtggccactcaggcctatagccccaaatctgcctggtaacccattatgtccccagagaatttgcatgggcggacaccctcatgccgggtctcagtaagggaaggggtgggaggcaaaaatatccctccccaccctgaatctccaccccctccccccagaaactgacacttggccttgtctaaggatgggttttcccaaaatccttctgaaaaaaacagaatttcaagagtcactccctccgggtctcagcctagaacatatgcagtatcccctgacgtccataggg

SEQ ID NO: 60 - DNA, 716 bp, Mus musculus
aaactggcacagtaatggcgggctgacagacaagggagtctgtagcacccgctgcctccgcccaccccttctccgagcaattaaaaggtgtttatgtggggctggcagtggcttctgcctcccttccattacgaacattaagagatcttgacccttccactttccccgctcttgaaaggagctgcagacacgtggagccaattaggcgcacgcgtgggcgccaagggcctgagcagctttttctccctgattgcggcgtttacagctgattattctcccctcacccaaacagtgctgcttcctggcaaggtgccacccagaggagccggctgggggcccctggggacaggggaggactggattagtaaatgggcatctatcgaatggctttcatatgtgtggctggaagggagaagggtagggccaggaatggtggcagcaagggcccaggtagcaatgagggttcttctaacccaccatttagggatagcgatcagaaaagggccctcgaggaggtgacctaaatgtgtgtagaagctgacggccactacacacacacacacacacacacacacacatacacaagcatccttgtccttggagtcggtcagcatgagcaagagaaagatgttcccagtggccatgagagtggagccctcctccctacttacatccaggttggatggccaggagatcctgagatccttcaagactcc

SEQ ID NO: 61 - DNA, 147 bp, Mus musculus
aagccacatcctgggtggaaatatatggcttcaattcccactcttccggatgacctctgtggggagccctggcttcaccttggtccagcttcatcccttagcctcgctgccaggaaggcagtgaggtcagaggctggtgctggcgtg

SEQ ID NO: 62 - DNA, 297 bp, Mus musculus
cctacctggtgcccgccaacatctgggggccatcctggccagcgccagcgtggtggtgaaggcactgtgcgccgtggtactgtttctctacctgctttccttcgctgtggacacgggctgcctggccgtcaccccaggctaccttttcccacccaacttctggatctggaccctggccacccacgggctcatggaacagcacgtgtgggacgtggccattagcctggccacagtggttgtggccgggcgattactggagcccctctggggagccttggagctgctcatcttcttctc

SEQ ID NO: 63 - DNA, 101 bp, Homo sapiens
aaacggacgggcctccgctgaaccagtgaggccccagacgtgcgcataaataacccctgcgtgctgcaccacctggggagagggggaggaccacggtaaat

SEQ ID NO: 64 - DNA, 101 bp, Homo sapiens
ggagcgagcgcatagcaaaagggacgcggggtccttttctctgccggtggcactgggtagctgtggccaggtgtggtactttgatggggcccagggctgga

SEQ ID NO: 65 - DNA, 101 bp, Homo sapiens
gctcaaggaagcgtcgcagggtcacagatctgggggaaccccggggaaaagcactgaggcaaaaccgccgctcgtctcctacaatatatgggagggggagg

SEQ ID NO: 66 - DNA, 547 bp, Homo sapiens
ttgagtacgttctggattactcataagaccttttttttttccttccgggcgcaaaaccgtgagctggatttataatcgccctataaagctccagaggcggtcaggcacctgcagaggagccccgccgctccgccgactagctgcccccgcgagcaacggcctcgtgatttccccgccgatccggtccccgcctccccactctgcccccgcctaccccggagccgtgcagccgcctctccgaatctctctcttctcctggcgctcgcgtgcgagagggaactagcgagaacgaggaagcagctggaggtgacgccgggcagattacgcctgtcagggccgagccgagcggatcgctgggcgctgtgcagaggaaaggcgggagtgcccggctcgctgtcgcagagccgaggtgggtaagctagcgaccacctggacttcccagcgcccaaccgtggcttttcagccaggtcctctcctcccgcggcttctcaaccaaccccatcccagcgccggccacccaacctcccgaaatgagtgcttcctgccc

SEQ ID NO: 67 - DNA, 101 bp, Homo sapiens
cagcagccgaaggcgctactaggaacggtaacctgttacttttccaggggccgtagtcgacccgctgcccgagttgctgtgcgactgcgcgcgcggggcta

SEQ ID NO: 68 - DNA, 201 bp, Homo sapiens
gagtgcaaggtgactgtggttcttctctggccaagtccgagggagaacgtaaagatatgggcctttttccccctctcaccttgtctcaccaaagtccctagtccccggagcagttagcctctttctttccagggaattagccagacacaacaacgggaaccagacaccgaaccagacatgcccgccccgtgcgccctcccc

SEQ ID NO: 69 - DNA, 199 bp, Homo sapiens
gctcgctgcctttcctccctcttgtctctccagagccggatcttcaaggggagcctccgtgcccccggctgctcagtccctccggtgtgcaggaccccggaagtcctccccgcacagctctcgcttctctttgcagcctgtttctgcgccggaccagtcgaggactctggacagtagaggccccgggacgaccgagctg

SEQ ID NO: 70 - DNA, 1351 bp, Homo sapiens
aaacggacgggcctccgctgaaccagtgaggccccagacgtgcgcataaataacccctgcgtgctgcaccacctggggagagggggaggaccacggtaaatggagcgagcgcatagcaaaagggacgcggggtccttttctctgccggtggcactgggtagctgtggccaggtgtggtactttgatggggcccagggctggagctcaaggaagcgtcgcagggtcacagatctgggggaaccccggggaaaagcactgaggcaaaaccgccgctcgtctcctacaatatatgggagggggaggttgagtacgttctggattactcataagaccttttttttttccttccgggcgcaaaaccgtgagctggatttataatcgccctataaagctccagaggcggtcaggcacctgcagaggagccccgccgctccgccgactagctgcccccgcgagcaacggcctcgtgatttccccgccgatccggtccccgcctccccactctgcccccgcctaccccggagccgtgcagccgcctctccgaatctctctcttctcctggcgctcgcgtgcgagagggaactagcgagaacgaggaagcagctggaggtgacgccgggcagattacgcctgtcagggccgagccgagcggatcgctgggcgctgtgcagaggaaaggcgggagtgcccggctcgctgtcgcagagccgaggtgggtaagctagcgaccacctggacttcccagcgcccaaccgtggcttttcagccaggtcctctcctcccgcggcttctcaaccaaccccatcccagcgccggccacccaacctcccgaaatgagtgcttcctgccccagcagccgaaggcgctactaggaacggtaacctgttacttttccaggggccgtagtcgacccgctgcccgagttgctgtgcgactgcgcgcgcggggctagagtgcaaggtgactgtggttcttctctggccaagtccgagggagaacgtaaagatatgggcctttttccccctctcaccttgtctcaccaaagtccctagtccccggagcagttagcctctttctttccagggaattagccagacacaacaacgggaaccagacaccgaaccagacatgcccgccccgtgcgccctccccgctcgctgcctttcctccctcttgtctctccagagccggatcttcaaggggagcctccgtgcccccggctgctcagtccctccggtgtgcaggaccccggaagtcctccccgcacagctctcgcttctctttgcagcctgtttctgcgccggaccagtcgaggactctggacagtagaggccccgggacgaccgagctg

SEQ ID NO: 71 - DNA, 2051 bp, Homo sapiens
ggaggaagccatcaactaaactacaatgactgtaagatacaaaattgggaatggtaacatattttgaagttctgttgacataaagaatcatgatattaatgcccatggaaatgaaagggcgatcaacactatggtttgaaaagggggaaattgtagagcacagatgtgttcgtgtggcagtgtgctgtctctagcaatactcagagaagagagagaacaatgaaattctgattggccccagtgtgagcccagatgaggttcagctgccaactttctctttcacatcttatgaaagtcatttaagcacaactaactttttttttttttttttttttttgagacagagtcttgctctgttgcccaggacagagtgcagtagtgactcaatctcggctcactgcagcctccacctcctaggctcaaacggtcctcctgcatcagcctcccaagtagctggaattacaggagtggcccaccatgcccagctaatttttgtatttttaatagatacgggggtttcaccatatcacccaggctggtctcgaactcctggcctcaagtgatccacctgcctcggcctcccaaagtgctgggattataggcgtcagccactatgcccaacccgaccaaccttttttaaaataaatatttaaaaaattggtatttcacatatatactagtatttacatttatccacacaaaacggacgggcctccgctgaaccagtgaggccccagacgtgcgcataaataacccctgcgtgctgcaccacctggggagagggggaggaccacggtaaatggagcgagcgcatagcaaaagggacgcggggtccttttctctgccggtggcactgggtagctgtggccaggtgtggtactttgatggggcccagggctggagctcaaggaagcgtcgcagggtcacagatctgggggaaccccggggaaaagcactgaggcaaaaccgccgctcgtctcctacaatatatgggagggggaggttgagtacgttctggattactcataagaccttttttttttccttccgggcgcaaaaccgtgagctggatttataatcgccctataaagctccagaggcggtcaggcacctgcagaggagccccgccgctccgccgactagctgcccccgcgagcaacggcctcgtgatttccccgccgatccggtccccgcctccccactctgcccccgcctaccccggagccgtgcagccgcctctccgaatctctctcttctcctggcgctcgcgtgcgagagggaactagcgagaacgaggaagcagctggaggtgacgccgggcagattacgcctgtcagggccgagccgagcggatcgctgggcgctgtgcagaggaaaggcgggagtgcccggctcgctgtcgcagagccgaggtgggtaagctagcgaccacctggacttcccagcgcccaaccgtggcttttcagccaggtcctctcctcccgcggcttctcaaccaaccccatcccagcgccggccacccaacctcccgaaatgagtgcttcctgccccagcagccgaaggcgctactaggaacggtaacctgttacttttccaggggccgtagtcgacccgctgcccgagttgctgtgcgactgcgcgcgcggggctagagtgcaaggtgactgtggttcttctctggccaagtccgagggagaacgtaaagatatgggcctttttccccctctcaccttgtctcaccaaagtccctagtccccggagcagttagcctctttctttccagggaattagccagacacaacaacgggaaccagacaccgaaccagacatgcccgccccgtgcgccctccccgctcgctgcctttcctccctcttgtctctccagagccggatcttcaaggggagcctccgtgcccccggctgctcagtccctccggtgtgcaggaccccggaagtcctccccgcacagctctcgcttctctttgcagcctgtttctgcgccggaccagtcgaggactctggacagtagaggccccgggacgaccgagctg

SEQ ID NO: 72 - DNA, 1878 bp, Unknown
  Description of Unknown Human or mouse regulatory element sequence
tcaacagggggacacttgggaaagaaggatggggacagagccgagaggactgttacacattagagaaacatcagtgactgtgccagctttggggtagactgcacaaaagccctgaggcagcacaggcaggatccagtctgctggtcccaggaagctaaccgtctcagacagagcacaaagcaccgagacatgtgccacaaggcttgtgtagagaggtcagaggacagcgtacaggtcccagagatcaaactcaacctcaccaggcttggcagcaagcctttaccaacccacccccaccccacccaccctgcacgcgcccctctcccctccccatggtctcccatggctatctcacttggccctaaaatgtttaaggatgacactggctgctgagtggaaatgagacagcagaagtcaacagtagattttaggaaagccagagaaaaaggcttgtgctgtttttagaaagccaagggacaagctaagatagggcccaagtaatgctagtatttacatttatccacacaaaacggacgggcctccgctgaaccagtgaggccccagacgtgcgcataaataacccctgcgtgctgcaccacctggggagagggggaggaccacggtaaatggagcgagcgcatagcaaaagggacgcggggtccttttctctgccggtggcactgggtagctgtggccaggtgtggtactttgatggggcccagggctggagctcaaggaagcgtcgcagggtcacagatctgggggaaccccggggaaaagcactgaggcaaaaccgccgctcgtctcctacaatatatgggagggggaggttgagtacgttctggattactcataagaccttttttttttccttccgggcgcaaaaccgtgagctggatttataatcgccctataaagctccagaggcggtcaggcacctgcagaggagccccgccgctccgccgactagctgcccccgcgagcaacggcctcgtgatttccccgccgatccggtccccgcctccccactctgcccccgcctaccccggagccgtgcagccgcctctccgaatctctctcttctcctggcgctcgcgtgcgagagggaactagcgagaacgaggaagcagctggaggtgacgccgggcagattacgcctgtcagggccgagccgagcggatcgctgggcgctgtgcagaggaaaggcgggagtgcccggctcgctgtcgcagagccgaggtgggtaagctagcgaccacctggacttcccagcgcccaaccgtggcttttcagccaggtcctctcctcccgcggcttctcaaccaaccccatcccagcgccggccacccaacctcccgaaatgagtgcttcctgccccagcagccgaaggcgctactaggaacggtaacctgttacttttccaggggccgtagtcgacccgctgcccgagttgctgtgcgactgcgcgcgcggggctagagtgcaaggtgactgtggttcttctctggccaagtccgagggagaacgtaaagatatgggcctttttccccctctcaccttgtctcaccaaagtccctagtccccggagcagttagcctctttctttccagggaattagccagacacaacaacgggaaccagacaccgaaccagacatgcccgccccgtgcgccctccccgctcgctgcctttcctccctcttgtctctccagagccggatcttcaaggggagcctccgtgcccccggctgctcagtccctccggtgtgcaggaccccggaagtcctccccgcacagctctcgcttctctttgcagcctgtttctgcgccggaccagtcgaggactctggacagtagaggccccgggacgaccgagctg

SEQ ID NO: 73 - DNA, 19 bp, Homo sapiens
atttacatttatccacaca

SEQ ID NO: 74 - DNA, 800 bp, Mus musculus
tgccgctggactctcttccaaggaactaggagaaccaagatccgtttttctgccaagggctgccccccccacgcccccaaccccctcaccccgatccccacagaaagaaatcttgaggtagctggagcttcttctgtgggtgtgacaggactgccattctcctctgtagtctgcagaagcctgccattccaccatttaaacctgtgactccaggccttaagcctgttgaaggtcgagtcccagaagggtcatatgtgcaactgcctagggagagttcccactcgcagggccaagaggagtcccccggtctgaggtgtgggggcggggacgtgcactgggcgctgggaccacggctggggctcaggactcgcgagcttggattcggatcggtttgcgcgagccagtagggcaggctccggggtgaacggggacgaggggcgcgcgggcacaggcgggcgcgtgaccgcggcgggggcgcgcggaggcgggccggccaaggagagggagggagggaatgagggagggagcgacaggggagggcggcgccggcaggttggcggcggccgctatttgagcgcaggtcccgggccaggcgctcaaagcgcttggagccagcgcggcggggagatcgctgcgcgcagcccgcagaggcgctgcggccagtgcagccccggaggccccgcgcggagaaggaggtggagaagaggccggctttccgcccgccgcccgcgcccccccacctccatcccgccgccgccgtcccccctccctccccgcggcgccgcatcttgaatggaaac

SEQ ID NO: 75 - DNA, 1335 bp, Artificial Sequence
  Description of Artificial Sequence Synthetic polynucleotide
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 76 - DNA, 2051 bp, Artificial Sequence; identical to SEQ ID NO: 71
  Description of Artificial Sequence Synthetic polynucleotide

SEQ ID NO: 77 - DNA, 24 bp, Artificial Sequence
  Description of Artificial Sequence Synthetic primer
gaatgtgggaaatcattcagtcgc

SEQ ID NO: 78 - DNA, 24 bp, Artificial Sequence
  Description of Artificial Sequence Synthetic primer
gcaagttatcctctcgtgagaagg

SEQ ID NO: 79 - DNA, 29 bp, Artificial Sequence
  Description of Artificial Sequence Synthetic probe
gcgacaacctggtgagacatcaacgcacc

SEQ ID NO: 80 - DNA, 22 bp, Artificial Sequence
  Description of Artificial Sequence Synthetic primer
gctgttatctcttgtgggctgt

SEQ ID NO: 81 - DNA, 22 bp, Artificial Sequence
  Description of Artificial Sequence Synthetic primer
aaactcatgggagctgccggtt

SEQ ID NO: 82 - DNA, 25 bp, Artificial Sequence
  Description of Artificial Sequence Synthetic probe
ccacacaaatctctccctggcattg

SEQ ID NO: 83 - DNA, 24 bp, Artificial Sequence
  Description of Artificial Sequence Synthetic primer
gaatgtgggaaatcattcagtcgc

SEQ ID NO: 84 - DNA, 24 bp, Artificial Sequence
  Description of Artificial Sequence Synthetic primer
gcaagttatcctctcgtgagaagg

SEQ ID NO: 85 - DNA, 29 bp, Artificial Sequence
  Description of Artificial Sequence Synthetic probe
gcgacaacctggtgagacatcaacgcacc

SEQ ID NO: 86 - DNA, 19 bp, Artificial Sequence
  Description of Artificial Sequence Synthetic primer
aaccgcatcgagctgaagg

SEQ ID NO: 87 - DNA, 22 bp, Artificial Sequence
  Description of Artificial Sequence Synthetic primer
gccatgatatagacgttgtggc

SEQ ID NO: 88 - DNA, 25 bp, Artificial Sequence
  Description of Artificial Sequence Synthetic probe
aggaggacggcaacatcctggggca

(19) United States
(12) Patent Application Publication
(10) Pub. No.: US 2022/0168449 A1
(43) Pub. Date: Jun. 2, 2022

(54) COMPOSITIONS AND METHODS FOR ADMINISTRATION OF THERAPEUTICS

(71) Applicant: Encoded Therapeutics, Inc., South San Francisco, CA (US)

(72) Inventors: Archana Belle, South San Francisco, CA (US); Stephanie Tagliatela, South San Francisco, CA (US)

(21) Appl. No.: 17/602,936

(22) PCT Filed: Apr. 10, 2020
(86) PCT No.: PCT/US2020/027682

§ 371 Date: Oct. 11, 2021

Related U.S. Application Data
(60) Provisional application No. 62/833,447, filed on Apr. 12, 2019.

(51) Int. Cl. A61K 48/00; A61K 9/00; A61P 25/28; C12N 15/86

(52) U.S. Cl. CPC A61K 48/0058; A61K 48/0075; C12N 15/86; A61K 9/0085; A61P 25/28; A61K 9/0019

(57) ABSTRACT

Provided herein are methods for administering a vector comprising a cell-type selective regulatory element. Such methods of administering comprise administration of one or more nucleic acid molecules to the central nervous system using methods such as intracerebroventricular administration, intrathecal administration, or intravenous administration.

The present application claims the benefit of priority to U.S. Provisional Patent Application No. 62/833,447, filed on Apr. 12, 2019, which is incorporated herein by reference.

### BACKGROUND OF THE DISCLOSURE

Gene therapy and antisense oligonucleotide therapies have long been recognized for their significant potential as treatments for neurological diseases or disorders. Instead of relying on surgery or drugs that treat only the symptoms of a neurological disease or disorder, patients, especially those with underlying genetic factors, can be treated by directly targeting the underlying disease/disorder cause. Furthermore, by targeting the underlying genetic causes of a neurological disease or disorder, gene therapy and antisense oligonucleotide based therapeutic approaches can provide sustained treatment over a longer period of time than standard pharmaceutical therapies and have the potential to effectively cure patients. Yet, despite this, clinical applications of gene therapy and antisense oligonucleotide based therapeutic approaches to neurological disorders still require improvement in several aspects. One area of concern for these therapies is the effective delivery of the therapeutic to the central nervous system. Vectors such as AAV9 have been shown to cross the blood brain barrier when administered intravenously in mice, but intravenous delivery of these vectors in larger animals is difficult due to the extremely high vector dose required for efficacy and the high transduction in peripheral organs which may could be associated with toxicity. Another route of administration, intraparenchymal injections, require lower doses of vector, and are effective in transducing the targeted region of the central nervous system (CNS). However, intraparenchymal injections may not be suitable for treatment of disorders which require delivery of the vector throughout the CNS.

Thus, there is a need to identify elements and methods of use thereof for targeting gene therapy or gene expression to a tissue or cell type of interest in the CNS, which can decrease off-target effects, increase therapeutic efficacy in the target tissue and/or cell type, and/or increase patient safety and tolerance by lowering the effective dose needed to achieve efficacy.

### SUMMARY OF THE DISCLOSURE

Provided herein are compositions and methods, that, in some embodiments, may be used for treatment of neuronal diseases such as Dravet syndrome.

In some embodiments, the disclosure provides a method of administering a vector to a primate, comprising intracerebroventricular (ICV) administration of a vector to the primate, wherein the vector comprises a cell-type selective regulatory element. In some embodiments, the disclosure provides a method of administering a vector to a primate, comprising intracerebroventricular (ICV) administration of a vector to the primate, wherein the vector comprises a regulatory element, wherein the regulatory element results in increased transgene expression by at least 2 fold as compared to expression of the transgene when operably linked to a CMV promoter. In some embodiments, the disclosure provides a method of administering a vector to a primate, comprising intracerebroventricular (ICV) administration of a vector to the primate, wherein the vector is administered unilaterally. In some embodiments, the disclosure provides a method of administering a vector to a primate, comprising intracerebroventricular (ICV) administration of a vector to the primate, wherein the vector is not a self-complementary AAV. In certain embodiments, the primate is a human. In certain embodiments, the primate is a non-human primate. In certain embodiments, the non-human primate is an old world monkey, an orangutan, a gorilla, a chimpanzee, a crab-eating macaque, a rhesus macaque or a pig-tailed macaque. In certain embodiments, the vector comprises a nucleotide sequence operably linked to a regulatory element. In certain embodiments, the regulatory element is selectively expressed in neuronal cells. In certain embodiments, the neuronal cells are selected from the group consisting of unipolar, bipolar, multipolar, or pseudounipolar neurons. In certain embodiments, the neuronal cells are GABAergic neurons. In certain embodiments, the regulatory element is selectively expressed in glial cells. In certain embodiments, the glial cells are selected from the group consisting of astrocytes, oligodendrocytes, ependymal cells, Schwann cells, and satellite cells. In certain embodiments, the regulatory element is selectively expressed in non-neuronal cells. In certain embodiments, the vector is administered to more than one ventricle of the brain. In certain embodiments, the vector is administered bilaterally. In certain embodiments, the vector is administered simultaneously. In certain embodiments, the vector is administered sequentially. In certain embodiments, each dose of the vector is administered at least 24 hours apart. In certain embodiments, the vector is administered to one ventricle of the brain. In certain embodiments, the primate further receives an intravenous administration of the vector. In certain embodiments, the primate further receives an intrathecal administration of the vector. In certain embodiments, the intrathecal administration comprises intrathecal cisternal administration or intrathecal lumbar administration. In certain embodiments, the vector comprises a nucleotide sequence encoding a polypeptide. In certain embodiments, the polypeptide is a DNA binding protein. In certain embodiments, the DNA binding protein is selected from the group consisting of a zinc finger protein (ZFP), a zinc finger nuclease (ZFN), or a transcription activator-like effector nuclease (TALEN). In certain embodiments, the nucleotide sequence is a codon-optimized variant and/or a fragment thereof. In certain embodiments, the vector comprises a nucleotide sequence encoding a guide RNA (gRNA). In certain embodiments, the vector comprises a nucleotide sequence encoding an interfering RNA (RNAi) that reduces expression of a target gene. In certain embodiments, the RNAi reduces expression of a target gene selected from the group consisting of SOD1, HTT, Tau, or alpha-synuclein. In certain embodiments, the vector comprises a nucleotide sequence encoding an antisense oligonucleotide that reduces expression of a target gene. In certain embodiments, the vector is selected from the group consisting of a lentivirus, retrovirus, plasmid, or herpes simplex virus (HSV). In certain embodiments, the vector is an adeno-associated viral (AAV) vector. In certain embodiments, the AAV is a single-stranded AAV. In certain embodiments, the AAV is a self-complementary AAV. In certain embodiments, the adeno-associated viral vector is any one of AAV1, scAAV1, AAV2, AAV3, AAV4, AAV5, scAAV5, AAV6, AAV7, AAV8, AAV9, scAAV9, AAV10, AAV11, AAV12, rh10, avian AAV, bovine AAV, canine AAV, equine AAV, primate AAV, non-primate AAV, or ovine AAV, or any hybrids thereof. In certain embodiments, the AAV vector is AAV5. In certain embodiments, the AAV vector is AAV9. In certain embodiments, the vector comprises a 5′ AAV inverted terminal repeat (ITR) sequence and a 3′ AAV ITR sequence. In certain embodiments, the vector is administered in a pharmaceutically acceptable carrier. In certain embodiments, the vector is administered in combination with a contrast agent. In certain embodiments, the vector is not administered in combination with a contrast agent. In certain embodiments, the administration is by route of injection. In certain embodiments, the administration is by route of infusion.

In some embodiments, the disclosure provides a method for expressing a gene of interest or a biologically active variant and/or fragment thereof comprising administering to a primate a therapeutically effective amount of an adeno-associated virus 1 (AAV1) vector or an adeno-associated virus 5 (AAV5) vector encoding the gene of interest, wherein the route of administration is selected from the group consisting of intravenous administration, intrathecal administration, intracerebroventricular administration, intraparenchymal administration, or combinations thereof. In certain embodiments, the primate is a human. In certain embodiments, the primate is a non-human primate. In certain embodiments, the non-human primate is an old world monkey, an orangutan, a gorilla, a chimpanzee, a crab-eating macaque, a rhesus macaque or a pig-tailed macaque. In certain embodiments, the AAV1 vector or AAV5 vector comprises a nucleotide sequence operably linked to a regulatory element. In certain embodiments, the regulatory element is cell-type selective. In certain embodiments, the regulatory element is selectively expressed in a neuronal cell. In certain embodiments, the neuronal cells are selected from the group consisting of unipolar, bipolar, multipolar, or pseudounipolar neurons. In certain embodiments, the neuronal cells are GABAergic neurons. In certain embodiments, the regulatory element is selectively expressed in glial cells. In certain embodiments, the glial cells are selected from the group consisting of astrocytes, oligodendrocytes, ependymal cells, Schwann cells, and satellite cells. In certain embodiments, the regulatory element is selectively expressed in non-neuronal cells. In certain embodiments, the AAV1 or AAV5 is administered to more than one ventricle of the brain. In certain embodiments, the AAV1 or AAV5 is administered bilaterally. In certain embodiments, the AAV1 or AAV5 is administered simultaneously. In certain embodiments, the AAV1 or AAV5 is administered sequentially. In certain embodiments, each dose of the AAV1 or AAV5 is administered at least 24 hours apart. In certain embodiments, the AAV1 or AAV5 is administered to one ventricle of the brain. In certain embodiments, the AAV1 or AAV5 comprises a nucleotide sequence encoding a polypeptide. In certain embodiments, the polypeptide is a DNA binding protein. In certain embodiments, the DNA binding protein is selected from the group consisting of a zinc finger protein (ZFP), a zinc finger nuclease (ZFN), or a transcription activator-like effector nuclease (TALEN). In certain embodiments, the nucleotide sequence is a codon-optimized variant and/or a fragment thereof. In certain embodiments, the vector comprises a nucleotide sequence encoding a guide RNA (gRNA). In certain embodiments, the AAV1 or AAV5 comprises a nucleotide sequence encoding an interfering RNA (RNAi) that reduces expression of a target gene. In certain embodiments, the RNAi reduces expression of a target gene selected from the group consisting of SOD1, HTT, Tau, or alpha-synuclein. In certain embodiments, the AAV1 or AAV5 comprises a nucleotide sequence encoding an antisense oligonucleotide that reduces expression of a target gene. In certain embodiments, the vector is selected from the group consisting of a lentivirus, retrovirus, plasmid, or herpes simplex virus (HSV). In certain embodiments, the AAV1 or AAV5 is administered in a pharmaceutically acceptable carrier. In certain embodiments, the vector is administered in combination with a contrast agent. In certain embodiments, the vector is not administered in combination with a contrast agent. In certain embodiments, the administration is by route of injection. In certain embodiments, the administration is by route of infusion.

In some embodiments, the disclosure provides a method to inhibit or treat one or more symptoms associated with a neuronal disease in a primate in need thereof, comprising administering an adeno-associated vector (AAV) selected from the group consisting of adeno-associated vector 1 (AAV1) or adeno-associated vector 5 (AAV5) to the primate, wherein the route of administration is selected from the group consisting of intravenous administration, intrathecal administration, intracerebroventricular administration, intraparenchymal administration, or combinations thereof. In certain embodiments, the neuronal disease is selected from the group consisting of a lysosomal storage disease, Dravet syndrome, Alzheimer's disease, Parkinson's disease, Huntington's disease, amyotrophic lateral sclerosis (ALS), spinal muscular atrophy (SMA), epilepsy, neurodegeneration, motor disorders, movement disorders, or mood disorders. In certain embodiments, the primate is a human. In certain embodiments, the primate is a non-human primate. In certain embodiments, the non-human primate is an old world monkey, an orangutan, a gorilla, a chimpanzee, a crab-eating macaque, a rhesus macaque or a pig-tailed macaque.

In some embodiments, the disclosure provides a method of administering a vector to a primate, comprising intracerebroventricular (ICV) administration of a vector to the primate, wherein the vector comprises a transgene, and wherein ICV administration results in increased transgene expression in the central nervous system (CNS) by at least 1.25-fold as compared to expression of the transgene when the vector is administered by any other route of administration. In certain embodiments, ICV administration produces at least 1.5-fold, 1.75-fold, 2-fold, 3-fold, 5-fold, 10-fold, 15-fold, 20-fold, 25-fold, 30-fold, 35-fold, 40-fold, 45-fold, 50-fold, 55-fold, 60-fold, 65-fold, 70-fold, or 75-fold, or at least 20-90 fold, 20-80 fold, 20-70 fold, 20-60 fold, 30-90 fold, 30-80 fold, 30-70 fold, 30-60 fold, 40-90 fold, 40-80 fold, 40-70 fold, 40-60 fold, 50-90 fold, 50-80 fold, 50-70 fold, 50-60 fold, 60-90 fold, 60-80 fold, 60-70 fold, 70-90 fold, 70-80 fold, 80-90 fold greater expression of the transgene sequence in the central nervous system (CNS) as compared to expression of the transgene when the vector is administered by any other route of administration. In some embodiments, ICV administration results in gene transfer throughout the brain. In certain embodiments, the gene transfer occurs in the frontal cortex, parietal cortex, temporal cortex, hippocampus, medulla, and occipital cortex. In certain embodiments, the gene transfer is dose dependent. In certain embodiments, the vector further comprises a cell-type selective regulatory element. In certain embodiments, the regulatory element is selectively expressed in the brain. In certain embodiments, the regulatory element is selectively expressed in the frontal cortex, parietal cortex, temporal cortex, hippocampus, medulla, and occipital cortex. In certain embodiments, the regulatory element is selectively expressed in the spine. In certain embodiments, the regulatory element is selectively expressed in the spinal cord and dorsal root ganglion. In certain embodiments, the regulatory element is selectively expressed in neuronal cells. In certain embodiments, the neuronal cells are selected from the group consisting of unipolar, bipolar, multipolar, or pseudounipolar neurons. In certain embodiments, the neuronal cells are GABAergic neurons. In certain embodiments, the regulatory element is selectively expressed in glial cells. In certain embodiments, the glial cells are selected from the group consisting of astrocytes, oligodendrocytes, ependymal cells, Schwann cells, and satellite cells. In certain embodiments, the regulatory element is selectively expressed in non-neuronal cells.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an exemplary representation of tissue slabs harvested from brain samples and indicates the location and number of tissue punches obtained for each of the frontal cortex, parietal cortex, temporal cortex, hippocampus, cerebellum, medulla and occipital cortex. For each type of tissue sample, tissue punches were obtained from both the right and left hemispheres and in some cases punches from two slabs were obtained.

FIG. 2 shows tissue distribution across the different tissue slabs and punches for animals treated with AAV9-CBA-eGFP-KASH administered at the high dose (1E+13 vector genome copies (vg)/animal) via unilateral intracerebroventricular (ICV), intracisterna magna (ICM) and intrathecal lumbar (IT-lumbar) routes of administration. Data is represented as vector copy number per diploid genome (VCN/diploid genome). Coronal section (CS) 2L represents the tissue punch from the left hemisphere of slab 2, CS 2R represents the tissue punch from the right hemisphere of slab 2, CS 8L represents the tissue punch from the top punch from the left hemisphere of slab 8 (see FIG. 1), CS 8L2 represents the tissue punch from the bottom punch from the left hemisphere of slab 8 (see FIG. 1, etc.).

FIG. 3 shows the average VCN/diploid genome in the brain for animals treated with AAV9-CBA-eGFP-KASH administered at the high dose (1E+13 vg/animal) via unilateral ICV, ICM and IT-lumbar routes of administration. Each data point represents the VC/diploid gDNA for each tissue punch and the horizontal bars represent the average VCN/diploid genome for all tissue punches for each route of administration. The VCN/diploid genome obtained with unilateral ICV administration was statistically significantly higher than the VCN/diploid genome obtained with either ICM or IT-lumbar administration.

FIG. 4 shows the VCN/diploid genome across the different regions of the brain (e.g., frontal cortex (FC), parietal cortex (PC), temporal cortex (TC), occipital cortex (OC), hippocampus (Hip), cerebellum (Cb), and medulla (Med)) for animals treated with AAV9-CBA-eGFP-KASH administered at the high dose (1E+13 vg/animal) via unilateral ICV, ICM and IT-lumbar routes of administration.

FIG. 5 shows the VCN/diploid genome in the spinal cord (SC), dorsal route ganglion (DRG), heart, liver, kidney and spleen tissue samples for animals treated with AAV9-CBA-eGFP-KASH administered at the high dose (1E+13 vg/animal) via unilateral ICV, ICM and IT-lumbar routes of administration. C2 refers to cervical region level 2, T1 and T8 refer to thoracic region levels T1 and T8, and L4 refers to lumbar region level 4 of the spinal cord.

FIG. 6 shows tissue distribution across the different tissue slabs and punches for animals treated with AAV9-CBA-eGFP-KASH or AAV9-SEQ ID 76-eGFP-WPRE administered at the low dose (2.4E+12 vg/animal) via unilateral intracerebroventricular (ICV), intracisterna magna (ICM), intrathecal lumbar (IT-lumbar), and intravenous (tail vein injection) routes of administration. Data is represented as VCN/diploid genome. For unilateral ICV administration, the data points represent that average of three treated animals. One animal was treated with AAV9-CBA-eGFP-KASH as described in Example 1 and two animals were treated with AAV9-SEQ ID 76-eGFP-WPRE as described in Example 2. Tissue punches are labeled as noted above for FIG. 2. One punch (noted on figure) obtained from the medulla tissue in slab 12 had very high levels of VCN/diploid genome, which was believed to be attributable to the proximity of the punch to the site of ICM administration.

FIG. 7 shows the average VCN/diploid genome in the brain for animals treated with AAV9-CBA-eGFP-KASH or AAV9-SEQ ID 76-eGFP-WPRE administered at the low dose (2.4E+12 vg/animal) via unilateral ICV, ICM, IT-lumbar and IV routes of administration. Each data point represents the VCN/diploid genome for each tissue punch and the horizontal bars represent the average VCN/diploid genome for all tissue punches for each route of administration. The VCN/diploid genome obtained with unilateral ICV administration was statistically significantly higher than the VCN/diploid genome obtained with ICM, IT-lumbar, and IV administration. For unilateral ICV administration, the data points represent that average of three treated animals. One animal was treated with AAV9-CBA-eGFP-KASH as described in Example 1 and two animals were treated with AAV9-SEQ ID 76-eGFP-WPRE as described in Example 2. The ICM punch with very high levels of VCN/diploid genome (as noted in FIG. 6) was excluded from this data set.

FIG. 8 shows the VCN/diploid genome across the different regions of the brain (e.g., frontal cortex (FC), parietal cortex (PC), temporal cortex (TC), occipital cortex (OC), hippocampus (Hip), cerebellum (Cb), and medulla (Med)) for animals treated with AAV9-CBA-eGFP-KASH or AAV9-SEQ ID 76-eGFP-WPRE administered at the low dose (2.4E+12 vg/animal) via unilateral ICV, ICM and IT-lumbar routes of administration. For unilateral ICV administration, the data points represent that average of three treated animals. One animal was treated with AAV9-CBA-eGFP-KASH as described in Example 1 and two animals were treated with AAV9-SEQ ID 76-eGFP-WPRE as described in Example 2.

FIG. 9 shows the VCN/diploid genome in the spinal cord (SC), dorsal route ganglion (DRG), heart, liver, kidney and spleen tissue samples for animals treated with AAV9-CBA-eGFP-KASH or AAV9-SEQ ID 76-eGFP-WPRE administered at the low dose (2.4E+12 vg/animal) via unilateral ICV, ICM, IT-lumbar, and IV routes of administration. For unilateral ICV administration, the data points represent that average of three treated animals. One animal was treated with AAV9-CBA-eGFP-KASH as described in Example 1 and two animals were treated with AAV9-SEQ ID 76-eGFP-WPRE as described in Example 2.

FIG. 10 shows tissue distribution across the different tissue slabs and punches for animals treated with AAV9-CBA-eGFP-KASH administered at the high dose (1E+13 vg/animal) via unilateral intracerebroventricular (ICV) or bilateral ICV administration. Data is represented as VCN/diploid genome. Tissue punches are labeled as noted above for FIG. 2.

FIG. 11 shows tissue distribution across the different tissue slabs and punches for animals treated with AAV9-CBA-eGFP-KASH or AAV9-SEQ ID 76-eGFP-WPRE administered at the high dose (˜2.4E+13 vg/animal) via unilateral intracerebroventricular (ICV) or bilateral ICV administration. Data is represented as VCN/diploid genome. For unilateral ICV administration, the data points represent that average of three treated animals. One animal was treated with AAV9-CBA-eGFP-KASH as described in Example 1 and two animals were treated with AAV9-SEQ ID 76-eGFP-WPRE as described in Example 2. Tissue punches are labeled as noted above for FIG. 2.

FIG. 12 shows the average VCN/diploid genome in the brain for animals treated with AAV9-CBA-eGFP-KASH or AAV9-SEQ ID 76-eGFP-WPRE administered at the high dose (ICV-H) of 1E+13 vg/animal or low dose (ICV-L) of 2.4E+12 vg/animal via unilateral ICV or bilateral ICV routes of administration. Each data point represents the VCN/diploid genome for each tissue punch and the horizontal bars represent the average VCN/diploid genome for all tissue punches for each route of administration. The VCN/diploid genome obtained with unilateral ICV administration was higher than the VCN/diploid genome obtained with bilateral ICV at both the high and low doses. For unilateral ICV administration at the low dose (ICV-L), the data points represent that average of three treated animals. One animal was treated with AAV9-CBA-eGFP-KASH as described in Example 1 and two animals were treated with AAV9-SEQ ID 76-eGFP-WPRE as described in Example 2.

FIG. 13 shows the VCN/diploid genome across the different regions of the brain (e.g., frontal cortex (FC), parietal cortex (PC), temporal cortex (TC), occipital cortex (OC), hippocampus (Hip), cerebellum (Cb), and medulla (Med)) for animals treated with AAV9-CBA-eGFP-KASH or AAV9-SEQ ID 76-eGFP-WPRE administered at the high dose (ICV-H) of 1E+13 vg/animal or low dose (ICV-L) of 2.4E+12 vg/animal via unilateral ICV or bilateral ICV routes of administration. For unilateral ICV administration, the data points represent that average of three treated animals. One animal was treated with AAV9-CBA-eGFP-KASH as described in Example 1 and two animals were treated with AAV9-SEQ ID 76-eGFP-WPRE as described in Example 2.

FIG. 14 shows the VCN/diploid genome in the spinal cord (SC), dorsal route ganglion (DRG), heart, liver, kidney and spleen tissue samples for animals treated with AAV9-CBA-eGFP-KASH or AAV9-SEQ ID 76-eGFP-WPRE administered at the high dose (ICV-H) of 1E+13 vg/animal or low dose (ICV-L) of 2.4E+12 vg/animal via unilateral ICV or bilateral ICV routes of administration. For unilateral ICV administration at the low dose (ICV-L), the data points represent that average of three treated animals. One animal was treated with AAV9-CBA-eGFP-KASH as described in Example 1 and two animals were treated with AAV9-SEQ ID 76-eGFP-WPRE as described in Example 2.

FIG. 15 shows green fluorescent protein (GFP) protein expression 4 weeks after dosing with AAV9 in the cortex, cerebellum, spinal cord, dorsal root ganglion (DRG), liver and heart as determined using an immunohistochemistry assay. AAV9 at high (HD=1E+13 vg/animal) or low dose (LD=˜2.4E+12 vg/animal) titer were administered by either unilateral or bilateral Intracerebroventricular (ICV), Intra-cisterna magna (ICM) injection, Intrathecal (IT-Lumbar) or Intravenous (IV). Images shown were contrast adjusted the same amount. A white 100 μm scale bar is shown in the lower left of each image along with the animal ID in the upper left.

FIG. 16 shows tissue distribution across the different tissue slabs and punches for animals treated with AAV9-CBA-eGFP-KASH, AAV9-SEQ ID 76-eGFP-WPRE, AAV5-CBA-eGFP-KASH or AAV1-CBA-eGFP-KASH administered at the low dose (˜2.4E+12 vg/animal) via unilateral intracerebroventricular (ICV) administration. Data is represented as VCN/diploid genome. For unilateral ICV administration with AAV9, the data points represent that average of three treated animals. One animal was treated with AAV9-CBA-eGFP-KASH as described in Example 1 and two animals were treated with AAV9-SEQ ID 76-eGFP-WPRE as described in Example 2. Tissue punches are labeled as noted above for FIG. 2.

FIG. 17 shows the average VCN/diploid genome in the brain for animals treated with AAV9-CBA-eGFP-KASH, AAV9-SEQ ID 76-eGFP-WPRE, AAV5-CBA-eGFP-KASH or AAV1-CBA-eGFP-KASH administered at the low dose (˜2.4E+12 vg/animal) via unilateral intracerebroventricular (ICV) administration. Each data point represents the VCN/diploid genome for each tissue punch and the horizontal bars represent the average VCN/diploid genome for all tissue punches for each serotype (e.g., AAV9, AAV5 and AAV1). For unilateral ICV administration with AAV9, the data points represent that average of three treated animals. One animal was treated with AAV9-CBA-eGFP-KASH as described in Example 1 and two animals were treated with AAV9-SEQ ID 76-eGFP-WPRE as described in Example 2.

FIG. 18 shows the VCN/diploid genome across the different regions of the brain (e.g., frontal cortex (FC), parietal cortex (PC), temporal cortex (TC), occipital cortex (OC), hippocampus (Hip), cerebellum (Cb), and medulla (Med)) for animals treated with AAV9-CBA-eGFP-KASH, AAV9-SEQ ID 76-eGFP-WPRE, AAV5-CBA-eGFP-KASH or AAV1-CBA-eGFP-KASH administered at the low dose (˜2.4E+12 vg/animal) via unilateral intracerebroventricular (ICV) administration. For unilateral ICV administration with AAV9, the data points represent that average of three treated animals. One animal was treated with AAV9-CBA-eGFP-KASH as described in Example 1 and two animals were treated with AAV9-SEQ ID 76-eGFP-WPRE as described in Example 2.

FIG. 19 shows the VCN/diploid genome in the spinal cord (SC), dorsal route ganglion (DRG), heart, liver, kidney and spleen tissue samples for animals treated with AAV9-CBA-eGFP-KASH, AAV9-SEQ ID 76-eGFP-WPRE, AAV5-CBA-eGFP-KASH or AAV1-CBA-eGFP-KASH administered at the low dose (˜2.4E+12 vg/animal) via unilateral intracerebroventricular (ICV) administration. For unilateral ICV administration with AAV9, the data points represent that average of three treated animals. One animal was treated with AAV9-CBA-eGFP-KASH as described in Example 1 and two animals were treated with AAV9-SEQ ID 76-eGFP-WPRE as described in Example 2.

FIG. 20 shows GFP expression 4 weeks after dosing with different AAV serotypes in the cortex, cerebellum, spinal cord, dorsal root ganglion (DRG), liver and heart using an immunohistochemical assay. Animals were dosed with AAV9, AAV5 or AAV1 vectors administered by unilateral Intracerebroventricular (ICV) injection as indicated. Images shown were contrast adjusted the same amount. A white 100 μm scale bar is shown in the lower left of each image along with the animal ID in the upper left.

FIG. 21 shows the VG/diploid genome in frontal cortex (FC), Rostral parietal cortex (Rostral PC), temporal cortex (TC), Caudal parietal cortex (Caudal PC), hippocampus (Hip), medulla (Med), and occipital cortex (OC) tissue samples for animals treated with AAV9 containing an expression cassette encoding eTFSCN1A under the control of a GABA selective regulatory element (AAV9-RE^{GABA}-eTF^{SCN1A}) administered at 4.8E+13 or 8E+13 vg/animal via unilateral intracerebroventricular (ICV) administration (Example 3 and Example 4). Each data point represents the VG/diploid genome for the tissue sample and the horizontal bars represent the average VG/diploid genome for all tissue samples for each animal.

FIG. 22 shows the transcripts/μg RNA in frontal cortex (FC), Rostral parietal cortex (Rostral PC), temporal cortex (TC), Caudal parietal cortex (Caudal PC), hippocampus (Hip), medulla (Med), and occipital cortex (OC) tissue samples for animals treated with AAV9-RE^{GABA}-eTF^{SCN1A} administered at 4.8E+13 or 8E+13 vg/animal via unilateral intracerebroventricular (ICV) administration (Example 3 and Example 4). Each data point represents the VG/diploid genome for the tissue sample and the horizontal bars represent the average VG/diploid genome for all tissue samples for each animal. Average transcripts for ARFGAP2 were 1.85E+6/μg RNA, and are indicated by the dashed upper boundary line. The detection limit is indicated by the dashed lower boundary line.

FIG. 23 shows vector biodistribution (VG/diploid genome) and transgene expression (transcripts/μg RNA) in peripheral tissue samples outside of the brain. The peripheral tissue samples shown are spinal cord C2/L4 (SC C2/L4), dorsal root ganglion C2/L4 (DRG C2/L4), liver, spleen, heart, kidney, lung, pancreas, and testis/ovary. Average VCN (vector biodistribution) and transcript (transgene expression) in the primate brain is indicated by a dashed line.

### DETAILED DESCRIPTION OF THE DISCLOSURE

### A. General Techniques

Unless otherwise defined herein, scientific and technical terms recited herein shall have the meanings that are commonly understood by those of ordinary skill in the art. Generally, nomenclature used in connection with, and techniques of, pharmacology, cell and tissue culture, molecular biology, cell and cancer biology, neurobiology, neurochemistry, virology, immunology, microbiology, genetics and protein and nucleic acid chemistry, described herein, are those well-known and commonly used in the art. In case of conflict, the present specification, including definitions, will control.

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Sambrook and Russell, Molecular Cloning: A Laboratory Manual, 3rd. ed., Cold Spring Harbor Laboratory Press, Cold Spring Harbor, N.Y. (2001); Ausubel et al., Current Protocols in Molecular Biology, John Wiley & Sons, NY (2002); Harlow and Lane Using Antibodies: A Laboratory Manual, Cold Spring Harbor Laboratory Press, Cold Spring Harbor, N.Y. (1998); Coligan et al., Short Protocols in Protein Science, John Wiley & Sons, NY (2003); Short Protocols in Molecular Biology (Wiley and Sons, 1999).

Enzymatic reactions and purification techniques are performed according to manufacturer's specifications, as commonly accomplished in the art or as described herein. The nomenclatures used in connection with, and the laboratory procedures and techniques of, analytical chemistry, biochemistry, immunology, molecular biology, synthetic organic chemistry, and medicinal and pharmaceutical chemistry described herein are those well known and commonly used in the art. Standard techniques are used for chemical syntheses, and chemical analyses.

### B. Definitions

Throughout this specification and embodiments, the word “comprise,” or variations such as “comprises” or “comprising,” will be understood to imply the inclusion of a stated integer or group of integers but not the exclusion of any other integer or group of integers.

It is understood that wherever embodiments are described herein with the language “comprising,” otherwise analogous embodiments described in terms of “consisting of” and/or “consisting essentially of” are also provided.

The term “including” is used to mean “including but not limited to.” “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

By way of example, “an element” means one element or more than one element.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the disclosure.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “AAV” is an abbreviation for adeno-associated virus and may be used to refer to the virus itself or a derivative thereof. The term covers all serotypes, subtypes, and both naturally occurring and recombinant forms, except where required otherwise. The abbreviation “rAAV” refers to recombinant adeno-associated virus. The term “AAV” includes AAV1, AAV2, AAV3, AAV4, AAV5, AAV6, AAV7, AAV8, AAV9, AAV10, AAV11, AAV12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. A “rAAV vector” as used herein refers to an AAV vector comprising a polynucleotide sequence not of AAV origin (i.e., a polynucleotide heterologous to AAV), typically a sequence of interest for the genetic transformation of a cell. In general, the heterologous polynucleotide is flanked by at least one, and generally by two, AAV inverted terminal repeat sequences (ITRs). An ITR sequence is a term well understood in the art and refers to relatively short sequences found at the termini of viral genomes which are in opposite orientation. An rAAV vector may either be single-stranded (ssAAV) or self-complementary (scAAV). An “AAV virus” or “AAV viral particle” refers to a viral particle composed of at least one AAV capsid protein and an encapsidated polynucleotide rAAV vector. If the particle comprises a heterologous polynucleotide (i.e., a polynucleotide other than a wild-type AAV genome such as a transgene to be delivered to a mammalian cell), it is typically referred to as an “rAAV viral particle” or simply an “rAAV particle”.

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% above and/or below a given value.

The terms “determining”, “measuring”, “evaluating”, “assessing”, “assaying”, “analyzing”, and their grammatical equivalents can be used interchangeably herein to refer to any form of measurement and include determining if an element is present or not (for example, detection). These terms can include both quantitative and/or qualitative determinations. Assessing may be relative or absolute.

An “expression cassette” refers to a nucleic molecule comprising one or more regulatory elements operably linked to a coding sequence (e.g., a gene or genes) for expression.

The term “effective amount” or “therapeutically effective amount” refers to that amount of a composition described herein that is sufficient to affect the intended application, including but not limited to disease treatment, as defined below. The therapeutically effective amount may vary depending upon the intended treatment application (in a cell or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in a target cell. The specific dose will vary depending on the particular composition chosen, the dosing regimen to be followed, whether it is administered in combination with other compounds, timing of administration, the tissue to which it is administered, and the physical delivery system in which it is carried.

A “fragment” of a nucleotide or peptide sequence refers to a fragment of the sequence that is shorter than the full-length or reference DNA or protein sequence.

The term “biologically active” as used herein when referring to a molecule such as a protein, polypeptide, nucleic acid, and/or polynucleotide means that the molecule retains at least one biological activity (either functional or structural) that is substantially similar to a biological activity of the full-length or reference protein, polypeptide, nucleic acid, and/or polynucleotide.

The term “in vitro” refers to an event that takes places outside of a subject's body. For example, an in vitro assay encompasses any assay run outside of a subject. In vitro assays encompass cell-based assays in which cells alive or dead are employed. In vitro assays also encompass a cell-free assay in which no intact cells are employed.

The term “in vivo” refers to an event that takes place in a subject's body.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally, at a chromosomal location that is different from its natural chromosomal location, or contains only coding sequences.

As used herein, “operably linked”, “operable linkage”, “operatively linked”, or grammatical equivalents thereof refer to juxtaposition of genetic elements, e.g., a promoter, an enhancer, a polyadenylation sequence, etc., wherein the elements are in a relationship permitting them to operate in the expected manner. For instance, a regulatory element, which can comprise promoter and/or enhancer sequences, is operatively linked to a coding region if the regulatory element helps initiate transcription of the coding sequence. There may be intervening residues between the regulatory element and coding region so long as this functional relationship is maintained.

A “pharmaceutically acceptable carrier” refers to an ingredient in a pharmaceutical formulation or composition, other than an active ingredient, which is nontoxic to a subject. A pharmaceutically acceptable carrier includes, but is not limited to, a buffer, excipient, stabilizer, or preservative.

The terms “pharmaceutical formulation” or “pharmaceutical composition” refer to a preparation which is in such form as to permit the biological activity of an active ingredient contained therein to be effective, and which contains no additional components which are unacceptably toxic to a subject to which the formulation would be administered.

The term “regulatory element” refers to a nucleic acid sequence or genetic element which is capable of influencing (e.g., increasing, decreasing, or modulating) expression of an operably linked sequence, such as a gene. Regulatory elements include, but are not limited to, promoter, enhancer, repressor, silencer, insulator sequences, an intron, UTR, an inverted terminal repeat (ITR) sequence, a long terminal repeat sequence (LTR), stability element, posttranslational response element, or a polyA sequence, or any combinations thereof. Regulatory elements can function at the DNA and/or the RNA level, e.g., by modulating gene expression at the transcriptional phase, post-transcriptional phase, or at the translational phase of gene expression; by modulating the level of translation (e.g., stability elements that stabilize mRNA for translation), RNA cleavage, RNA splicing, and/or transcriptional termination; by recruiting transcriptional factors to a coding region that increase gene expression; by increasing the rate at which RNA transcripts are produced, increasing the stability of RNA produced, and/or increasing the rate of protein synthesis from RNA transcripts; and/or by preventing RNA degradation and/or increasing its stability to facilitate protein synthesis. In some embodiments, a regulatory element refers to an enhancer, repressor, promoter, or any combinations thereof, particularly an enhancer plus promoter combination or a repressor plus promoter combination. In some embodiments, the regulatory element is derived from a human sequence.

The terms “subject” and “individual” are used interchangeably herein to refer to a vertebrate, preferably a mammal, more preferably a human. The methods described herein can be useful in human therapeutics, veterinary applications, and/or preclinical studies in animal models of a disease or condition.

As used herein, the terms “treat”, “treatment”, “therapy” and the like refer to obtaining a desired pharmacologic and/or physiologic effect, including, but not limited to, alleviating, delaying or slowing progression, reducing effects or symptoms, preventing onset, preventing reoccurrence, inhibiting, ameliorating onset of a diseases or disorder, obtaining a beneficial or desired result with respect to a disease, disorder, or medical condition, such as a therapeutic benefit and/or a prophylactic benefit. “Treatment,” as used herein, covers any treatment of a disease in a mammal, particularly in a human, and includes: (a) preventing the disease from occurring in a subject which may be predisposed to the disease or at risk of acquiring the disease but has not yet been diagnosed as having it; (b) inhibiting the disease, i.e., arresting its development; and (c) relieving the disease, i.e., causing regression of the disease. A therapeutic benefit includes eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. In some cases, for prophylactic benefit, the compositions are administered to a subject at risk of developing a particular disease, or to a subject reporting one or more of the physiological symptoms of a disease, even though a diagnosis of this disease may not have been made. The methods of the present disclosure may be used with any mammal. In some cases, the treatment can result in a decrease or cessation of symptoms. A prophylactic effect includes delaying or eliminating the appearance of a disease or condition, delaying or eliminating the onset of symptoms of a disease or condition, slowing, halting, or reversing the progression of a disease or condition, or any combination thereof.

A “variant” of a nucleotide sequence refers to a sequence having a genetic alteration or a mutation as compared to the most common wild-type DNA sequence (e.g., cDNA or a sequence referenced by its GenBank accession number) or a specified reference sequence.

A “vector” as used herein refers to a nucleic acid molecule that can be used to mediate delivery of another nucleic acid molecule to which it is linked into a cell where it can be replicated or expressed. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Other examples of vectors include plasmids, viral vectors, and cosmids.

In general, “sequence identity” or “sequence homology”, which can be used interchangeably, refer to an exact nucleotide-to-nucleotide or amino acid-to-amino acid correspondence of two polynucleotides or polypeptide sequences, respectively. Two or more sequences (polynucleotide or amino acid) can be compared by determining their “percent identity”, also referred to as “percent homology”. The percent identity to a reference sequence (e.g., nucleic acid or amino acid sequence) may be calculated as the number of exact matches between two optimally aligned sequences divided by the length of the reference sequence and multiplied by 100. Conservative substitutions are not considered as matches when determining the number of matches for sequence identity. It will be appreciated that where the length of a first sequence (A) is not equal to the length of a second sequence (B), the percent identity of A:B sequence will be different than the percent identity of B:A sequence. Sequence alignments, such as for the purpose of assessing percent identity, may be performed by any suitable alignment algorithm or program, including but not limited to the Needleman-Wunsch algorithm (see, e.g., the EMBOSS Needle aligner available on the world wide web at ebi.ac.uk/Tools/psa/embossneedle/), the BLAST algorithm (see, e.g., the BLAST alignment tool available on the world wide web at blast.ncbi.nlm.nih.gov/Blast.cgi), the Smith-Waterman algorithm (see, e.g., the EMBOSS Water aligner available on the world wide web at ebi.ac.uk/Tools/psa/embosswater/), and Clustal Omega alignment program (see e.g., the world wide web at clustal.org/omega/and F. Sievers et al., Mol Sys Biol. 7: 539 (2011)). Optimal alignment may be assessed using any suitable parameters of a chosen algorithm, including default parameters. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin and Altschul, Proc. Natl. Acad. Sci. USA 90:5873-5877 (1993); and Altschul et al., Nucleic Acids Res. 25:3389-3402 (1997).

Unless otherwise indicated, all terms used herein have the same meaning as they would to one skilled in the art and the practice of the present invention will employ, conventional techniques of molecular biology, microbiology, and recombinant DNA technology, which are within the knowledge of those of skill of the art.

### C. Nucleic Acid Constructs

In some embodiments, the present disclosure relates to methods of administering a vector comprising a cell-type selective regulatory element. In some embodiments, the vector comprises a regulatory element. In some embodiments, the regulatory element results in increased transgene expression by at least 2 fold as compared to expression of the transgene when operably linked to a CMV promoter. In some embodiments, the methods comprise administering vectors (e.g. AAV9) comprising a nucleotide sequence (e.g. a nucleotide sequence encoding a polypeptide) operably linked to a regulatory element. Thus, in some aspects, provided herein are nucleic acid components and compositions useful for practicing the methods of the present disclosure.

In some embodiments, the nucleic acid is a DNA molecule. In some embodiments, the nucleic acid is an RNA molecule. In some embodiments, the nucleic acid is a DNA molecule in any of the vectors disclosed herein. In some embodiments, the nucleic acid molecule comprises any of the transgenes disclosed herein. In some embodiments, the nucleic acid molecule comprises any of the regulatory elements disclosed herein. In some embodiments, the nucleic acid is a DNA molecule comprising any of the transgenes disclosed herein and any of the regulatory elements disclosed herein. In some embodiments, the nucleic acid molecule is an RNA nucleic acid molecule comprising any of the transgenes disclosed herein. In some embodiments, the RNA molecule is transcribed from any of the DNA molecules disclosed herein (e.g., a DNA molecule comprising any of the transgenes and regulatory elements disclosed herein). In some embodiments, the RNA molecule is transcribed from any of the DNA molecules disclosed herein (e.g., a DNA molecule comprising any of the transgenes and regulatory elements disclosed herein), wherein the RNA molecule comprises a transgene sequence.

1. Transgenes

In some embodiments, any of the nucleic acid molecules provided herein that can be used according to the present methods comprises a transgene sequence operably linked to a regulatory element for use in the methods disclosed herein. In some embodiments, the transgenes of the present compositions and methods may be used to inhibit or treat one or more symptoms associated with a neuronal disease (e.g. Dravet syndrome).

Any transgene of interest can be designed and used in the present methods. In some embodiments, the transgene comprises a modified nucleotide sequence (e.g., alternative codons) as compared to a reference nucleotide sequence. In some embodiments, the transgene can be designed to have certain beneficial properties, e.g., the expressed transgene specifically expresses in a subset of cells which are therapeutically relevant to a disease (e.g. Alzheimer's disease). In some embodiments, the transgene is a DNA nucleic acid molecule. In some embodiments, the transgene is an RNA nucleic acid molecule that has been transcribed from any of the DNA nucleic acid molecules described herein.

In some embodiments, the transgene encodes a therapeutic protein. In some embodiments, expression of the therapeutic protein in a subject (e.g., a primate) reduces the risk of developing a disease or disorder (e.g., a neurological disease or disorder). In some embodiments, the transgene encodes a wildtype version of a protein and may be administered to a subject expressing a mutant version of a protein. In some embodiments, the transgene encodes a wildtype version of a protein and may be administered to a subject in order to increase expression levels of the wildtype version of the protein in the subject. In some embodiments, the transgene encodes a mutant form of a protein, wherein the mutant protein is associated with increased or constitutive activity as compared to a wildtype version of the protein. In some embodiments, the transgene encodes a specific isoform of a protein, wherein expression of the specific protein isoform in a subject is associated with reduced risk of development of a disease or disorder (e.g., human apolipoprotein E2). In some embodiments, the specific protein isoform is administered to a subject expressing a harmful isoform of the same protein (e.g., human apolipoprotein E4).

In some embodiments, the transgene comprises a sequence encoding a polypeptide. In some embodiments, the transgene comprises a sequence encoding a gene-editing polypeptide. In some embodiments, the polypeptide encoded by the transgene is a DNA binding protein. In some embodiments, the DNA binding protein is selected from the group consisting of a zinc finger protein (ZFP), a zinc finger nuclease (ZFN), and a transcription activator-like effector nuclease (TALEN). In some embodiments, the transgene comprises a nucleotide sequence that is a codon-optimized variant and/or fragment thereof.

In some embodiments, the transgene comprises a sequence encoding a guide RNA (gRNA). In some embodiments, the transgene comprises a sequence encoding a gRNA operably linked to a regulatory element. In some embodiments, the guide RNA can be used in combination with an RNA-guided DNA binding agent (e.g., Cas nuclease) and a donor construct. In some embodiments, the donor construct can be used with a gene editing system (e.g., CRISPR/Cas system; ZFN system; TALEN system).

As used herein, the terms “guide RNA” and “gRNA” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “gRNA” refers to both single guide RNA or dual guide RNA formats. The trRNA may be a naturally-occurring sequence, or a trRNA sequence with modifications or variations compared to naturally-occurring sequences. Guide RNAs, such as sgRNAs or dgRNAs, can include modified RNAs as described herein.

In some embodiments, the transgene comprises a sequence encoding an antisense oligonucleotide. In some embodiments, the transgene comprises a sequence encoding an antisense oligonucleotide operably linked to a regulatory element. In some embodiments, the antisense oligonucleotide reduces expression of a target gene. In some embodiments, the transgene encodes an antisense oligonucleotide that targets a gene associated with a CNS disorder, such as, for example, a voltage-gated ion channel or a subunit thereof. Voltage gated ion channels include sodium channels, calcium channels, potassium channels, and proton channels. Examples of voltage gated sodium channel subunits include SCN1B (NM_001037.4), SCN1A (NM_001165963.1), SCN2B, (NM_004588.4), SCN2A, SNC8A, KV3.1, KV3.2, or KV3.3. In some embodiments, the transgene encodes an antisense oligonucleotide that targets a pre-mRNA of SCN1A or SCN8A, or a natural antisense polynucleotide of SCN1A.

In some embodiments, the application provides a transgene encoding an antisense oligonucleotide that targets or is capable of upregulating a neurotransmitter regulator. A neurotransmitter regulator may be involved in regulating production or release of a neurotransmitter in the CNS. For example, a neurotransmitter regulator may assist with synaptic fusion to release neurotransmitters. An example of a neurotransmitter regulator is STXBP1 (NM_001032221.3).

In some embodiments, the application provides transgenes encoding an antisense oligonucleotide operably linked to a cell-type selective regulatory element, wherein the antisense oligonucleotide is capable of upregulating the expression or function of a gene of interest such as a voltage-gated ion channel or a subunit thereof. In some embodiments, the application provides transgenes encoding antisense oligonucleotides that promote splicing of a voltage gated sodium channel pre-mRNA that has a retained intron. In another embodiment, the application provides transgenes encoding antisense oligonucleotides that modulate the splicing of a voltage gated sodium channel pre-mRNA. In another embodiment, the application provides transgenes encoding antisense oligonucleotides that are targeted to natural antisense polynucleotides of a voltage gated sodium channel. In some embodiments, the transgene encodes an antisense oligonucleotide that is capable of upregulating the expression or function of SCN1A. In some embodiments, the transgene encodes an antisense oligonucleotide that is capable of downregulating the expression or function of SCN8A.

In some embodiments, the application provides transgenes encoding an antisense oligonucleotide that promotes exon skipping, exon inclusion, removal of a retained intron, or eradication, degradation or inactivation of deleterious mRNAs of a target gene, or eradication, degradation or inactivation of a natural antisense polynucleotide of a target gene. In some embodiments, the target gene is SCN1A or SCN8A. Various antisense oligonucleotides suitable for use in connection with the compositions and methods disclosed herein may be found, for example, in US 2017/0240904, U.S. Pat. No. 9,771,579, WO 2017/106377, U.S. Pat. No. 9,976,143, and WO 2017/106382.

As used herein, the term “antisense oligonucleotide” refers to oligonucleotides (e.g. RNA, DNA, mimetic, chimera, analogs or homologs thereof), ribozymes, external guide sequence (EGS) oligonucleotides, single- or double-stranded RNA interference (RNAi) compounds such as short interfering RNA (siRNA), micro interfering RNA (miRNA), a small, temporal RNA (stRNA), a short, hairpin RNA (shRNA), small RNA-induced gene activation (RNAa), small activating RNA (saRNA), or a small nuclear RNA (snRNA) such as a U1 or U7 snRNA, and other oligomeric compounds which hybridize to at least a portion of the target nucleic acid and modulate its function. As such, an antisense oligonucleotide may be DNA, RNA, DNA-like, RNA-like, or mixtures thereof, or may be mimetics of one or more of these. Antisense oligonucleotides may be single-stranded, double-stranded, circular or hairpin oligomeric compounds and may contain structural elements such as internal or terminal bulges, mismatches or loops. Double stranded antisense oligonucleotides can be formed by hybridizing two strands to form a wholly or partially double-stranded oligonucleotide or by a single strand with sufficient self-complementarity to allow for hybridization and formation of a fully or partially double-stranded oligonucleotide. The two strands can be linked internally leaving free 3′ or 5′ termini or can be linked to form a continuous hairpin structure or loop. The hairpin structure may contain an overhang on either the 5′ or 3′ terminus producing an extension of single stranded character. The double stranded antisense oligonucleotides optionally can include overhangs on the ends. When formed from only one strand, dsRNA can take the form of a self-complementary hairpin-type molecule that doubles back on itself to form a duplex. Thus, the dsRNAs can be fully or partially double stranded. Specific modulation of gene expression can be achieved by stable expression of antisense RNA oligonucleotides in transgenic cell lines or via gene therapy. When formed from two strands, or a single strand that takes the form of a self-complementary hairpin-type molecule doubled back on itself to form a duplex, the two strands (or duplex-forming regions of a single strand) are complementary RNA strands that base pair in Watson-Crick fashion. In some embodiments, antisense oligonucleotides provided herein are single stranded RNA oligonucleotides. In certain embodiments, the single stranded antisense RNAs are provided as part of a modified huU7 snRNA molecule.

In various embodiments, an antisense oligonucleotide encoded by a transgene as provided herein may be fully or partially complementary to a target gene or sequence. In certain embodiments, the homology, sequence identity or complementarity, between the antisense oligonucleotide and target sequence is from about 40% to about 60%. In some embodiments, homology, sequence identity or complementarity, is from about 60% to about 70%. In some embodiments, homology, sequence identity or complementarity, is from about 70% to about 80%. In some embodiments, homology, sequence identity or complementarity, is from about 80% to about 90%. In some embodiments, homology, sequence identity or complementarity, is about 90%, about 92%, about 94%, about 95%, about 96%, about 97%, about 98%, about 99% or about 100%.

In some embodiments, the transgene comprises a sequence encoding an RNA (RNAi). In some embodiments, the transgene comprises a sequence encoding an RNA) operably linked to a regulatory element. In some embodiments, the RNAi reduces expression of a target gene. In some embodiments, the RNAi reduces expression of a target gene selected from the group consisting of SOD1, HTT, Tau, or alpha-synuclein. As used herein, the term ““RNAi” refers to an RNA (or analog thereof), having sufficient sequence complementarity to a target RNA to direct RNA interference.

2. Regulatory Elements

Regulatory elements can function at the DNA and/or the RNA level. Regulatory elements can function to modulate gene expression selectivity in a cell type of interest. Regulatory elements can function to modulate gene expression at the transcriptional phase, post-transcriptional phase, or at the translational phase of gene expression. Regulatory elements include, but are not limited to, promoter, enhancer, intronic, or other non-coding sequences. At the RNA level, regulation can occur at the level of translation (e.g., stability elements that stabilize mRNA for translation), RNA cleavage, RNA splicing, and/or transcriptional termination. In some cases, regulatory elements can recruit transcriptional factors to a coding region that increase gene expression selectivity in a cell type of interest. In some cases, regulatory elements can increase the rate at which RNA transcripts are produced, increase the stability of RNA produced, and/or increase the rate of protein synthesis from RNA transcripts.

Regulatory elements are nucleic acid sequences or genetic elements which are capable of influencing (e.g., increasing) expression of a gene (e.g., a reporter gene such as EGFP or luciferase; a transgene; or a therapeutic gene) in one or more cell types or tissues. In some cases, a regulatory element can be a transgene, an intron, a promoter, an enhancer, UTR, an inverted terminal repeat (ITR) sequence, a long terminal repeat sequence (LTR), stability element, posttranslational response element, or a polyA sequence, or a combination thereof. In some cases, the regulatory element is a promoter, an enhancer, an intronic sequence, or a combination thereof. In some cases, the regulatory element is derived from a human sequence (e.g., hg19).

In some cases, a regulatory element of this disclosure results in high or increased expression of an operably linked transgene, wherein the high or increased expression is determined as compared to a control, e.g., a constitutive promoter, a CMV promoter, CAG, super core promoter (SCP), TTR promoter, Proto 1 promoter, UCL-HLP promoter, minCMV, EFS, or CMVe promoter. Other controls that can be used to determine high or increased transgene expression by a regulatory element disclosed herein include buffer alone or vector alone. In some cases, a positive control refers to a RE with known expression activity, such as SEQ ID NO: 39, which can be used for comparison. In some cases, a regulatory element drives comparable or higher transgene expression as comparable to a positive control (e.g., SEQ ID NO: 39 or a known promoter operably linked to the transgene).

In certain embodiments, the vector comprises a nucleotide sequence operably linked to a regulatory element. In certain embodiments, the nucleotide sequence is operably linked to a regulatory element having less than or equal to 400 base pairs (bp), 300 bp, 250 bp, 200 bp, 150 bp, 140 bp, 130 bp, 120 bp, 110 bp, 100 bp, 70 bp, or 50 bp. In certain embodiments, the regulatory element is any one of or combination of: any one of SEQ ID NOs: 1-29, CBA, CMV, SCP, SERpE_TTR, Proto1, minCMV, UCL-HLP, CMVe, CAG, or EFS. In certain embodiments, the regulatory element is any one of or combination of SEQ ID NO: 31, SEQ ID NO: 33, CBA, or minCMV. In certain embodiments, the regulatory element is SEQ ID NO: 33. In certain embodiments, the regulatory element is CBA. In certain embodiments, the regulatory element is minCMV. In certain embodiments, a vector disclosed herein comprises a promoter having any one of SEQ ID NOs: 1-40 (as shown below in Tables 5 and 6) operably linked to any transgene e.g., a DNA binding protein. In certain embodiments, the regulatory element is cell-type selective. In certain embodiments, the regulatory element is selectively expressed in neuronal cells. In certain embodiments, the regulatory element is selectively expressed in neuronal cells selected from the group consisting of unipolar, bipolar, multipolar, or pseudounipolar neurons. In certain embodiments, the regulatory element is selectively expressed in GABAergic neurons. In certain embodiments, the regulatory element is selectively expressed in glial cells. In certain embodiments, the glial cell is any one of the following glial cell types: astrocytes, oligodendrocytes, ependymal cells, Schwann cells, or satellite cells. In certain embodiments, the regulatory element is selectively expressed in microglia cells. In certain embodiments, the regulatory element is selectively expressed in non-neuronal cells.

In some embodiments, the regulatory element is derived from a human regulatory element. In some embodiments, a sequence is deemed to be human derived it has at least 80%, at least 85%, at least 90%, at least 91%, at least 92%, at least 93%, at least 94%, at least 95%, at least 96%, at least 97%, at least 98%, at least 99% or 100% identity to a human sequence. In some cases, a regulatory element contains a human derived sequence and a non-human derived sequence such that overall the regulatory element has low sequence identity to the human genome, while a part of the regulatory element has 100% sequence identity (or local sequence identity) to a sequence in the human genome.

In certain embodiments, the present disclosure provides a plurality of regulatory elements, that can be operably linked to any transgene to increase or to improve selectivity of the transgene expression in the CNS, e.g., in PV neurons. By increasing selectivity of gene expression using one or more regulatory elements disclosed herein, one can improve the efficacy of a gene therapy, decrease the effective dose needed to result in a therapeutic effect, minimize adverse effects or off-target effect, and/or increase patient safety and/or tolerance.

In one aspect, one or more regulatory elements can be operably linked to any transgene in an expression cassette to modulate gene expression in a cell, such as targeting expression of the transgene in a target cell type or tissue (e.g., PV cells) over one or more non-target cell type or tissue (e.g., non-PV CNS cell-types). In some cases, targeting expression of the transgene in a target cell type or tissue includes increased gene expression in the target cell type or tissue.

In some cases, operably linking one or more regulatory elements to a gene results in targeted expression of the gene in a target tissue or cell type in the CNS, such as a parvalbumin (PV) neuron. In some cases, one or more regulatory elements (e.g., SEQ ID NOs: 41-75, or a functional fragment or a combination thereof, or sequences having at least 80%, at least 90%, at least 95%, or at least 99% sequence identity thereto) increase selectivity of gene expression in a target tissue or cell type in the CNS, such as PV neurons. In some cases, a gene therapy comprises one or more regulatory elements disclosed herein, wherein the regulatory elements are operably linked to a transgene and drive selective expression of the transgene in PV neurons.

In some cases, selective expression of a gene in PV neurons is used to treat a disease or condition associated with a haploinsufficiency and/or a genetic defect in an endogenous gene, wherein the genetic defect can be a mutation in the gene or dysregulation of the gene. Such genetic defect can result in a reduced level of the gene product and/or a gene product with impaired function and/or activity. In some cases, an expression cassette comprises a gene, a subunit, a variant or a functional fragment thereof, wherein gene expression from the expression cassette is used to treat the disease or condition associated with the genetic defect, impaired function and/or activity, and/or dysregulation of the endogenous gene. In some cases, the disease or condition is Dravet syndrome, Alzheimer's disease, epilepsy, neurodegeneration, tauopathy, neuronal hypoexcitability and/or seizures.

In some cases, any one or more of the regulatory elements disclosed herein result in increased selectivity in gene expression in a parvalbumin cell. In some cases, regulatory elements disclosed herein are PV-cell-selective. In some cases, PV cell selective regulatory elements are associated with selective gene expression in PV cells more than expression in non-PV CNS cell-types. In some cases, PV cell selective regulatory elements as associated with reduced gene expression in non-PV CNS cell types. Non-limiting examples of regulatory elements include SEQ ID NOs: 41-75, as provided in Table 7.

In certain embodiments, the vector comprises a nucleotide sequence operably linked to a regulatory element, wherein the regulatory element results in increased transgene expression by at least 2 fold as compared to expression of the transgene when operably linked to a CMV promoter. In certain embodiments, the promoter sequence produces at least 5-fold, 10-fold, 15-fold, 20-fold, 25-fold, 30-fold, 35-fold, 40-fold, 45-fold, 50-fold, 55-fold, 60-fold, 65-fold, 70-fold, or 75-fold, or at least 20-90 fold, 20-80 fold, 20-70 fold, 20-60 fold, 30-90 fold, 30-80 fold, 30-70 fold, 30-60 fold, 40-90 fold, 40-80 fold, 40-70 fold, 40-60 fold, 50-90 fold, 50-80 fold, 50-70 fold, 50-60 fold, 60-90 fold, 60-80 fold, 60-70 fold, 70-90 fold, 70-80 fold, 80-90 fold greater expression of the transgene sequence in a mammalian cell relative to the level of expression of the same transgene sequence from the CMV promoter in the same type of mammalian cell. In certain embodiments, the promoter sequence drives expression of the transgene sequence in a high percentage of neuronal cells, e.g., at least 20%, 25%, 30%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or greater, or at least 20-90%, 20-80%, 20-70%, 30-90%, 30-80%, 30-70%, 40-90%, 40-80%, 40-70%, 50-90%, 50-80%, 50-70%, 60-90%, 60-80%, 60-70%, 70-90%, 70-80%, 80-100%, 80-95%, 80-90%, 90-100%, or 90-95% of GABAergic cells containing the vector express the transgene. In certain embodiments, the promoter sequence drives expression of the transgene in a high percentage of glial cells, e.g., at least 20%, 25%, 30%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or greater, or at least 20-90%, 20-80%, 20-70%, 30-90%, 30-80%, 30-70%, 40-90%, 40-80%, 40-70%, 50-90%, 50-80%, 50-70%, 60-90%, 60-80%, 60-70%, 70-90%, 70-80%, 80-100%, 80-95%, 80-90%, 90-100%, or 90-95% of oligodendrocytes containing the vector express the transgene.

In some aspects, an AAV expression cassette comprises a human-derived regulatory element of no more than 120 bp operably linked to a transgene of at least 3 kb, wherein the regulatory element results in increased transgene expression by at least 2 fold as compared to expression of the transgene when operably linked to a CMV promoter. In some cases, the increased transgene expression is at least 50 fold. In some cases, the increased transgene expression is at least 100 fold. In some cases, the increased transgene expression occurs in at least 2 different cell types (e.g., excitatory neurons and inhibitory neurons). In some cases, the increased transgene expression occurs in at least 3 different cell types (e.g., excitatory neurons, inhibitory neurons, and liver cells).

In some cases, such high expression of the transgene in a cell or in vivo is relative to expression of the transgene without said regulatory elements, wherein expression of the transgene with the regulatory elements is at least 1.5 fold, at least 2 fold, at least 3 fold, at least 4 fold, at least 5 fold, at least 6 fold, at least 7 fold, at least 8 fold, at least 9 fold, at least 10 fold, at least 15 fold, at least 20 fold, at least 25 fold, at least 50 fold, at least 100 fold, at least 150 fold, at least 200 fold, at least 250 fold, at least 300 fold, at least 400 fold, at least 500 fold, at least 600 fold, at least 700 fold, at least 800 fold, at least 900 fold, at least 1000 fold, at least 1010 fold, at least 1020 fold, at least 1030 fold, at least 1040 fold, or at least 1050 fold as compared to transgene expression without the regulatory elements, or as compared to transgene expression by a negative control (e.g., buffer alone, vector alone, or a vector comprising a sequence known to have no expression activity).

In some cases, one or more regulatory elements result in high transgene expression in at least 2, at least 3, at least 4, at least 5, at least 6, at least 7, at least 8, at least 9, or at least 10 different cell types. In some cases, one or more regulatory elements of this disclosure are operably linked to a transgene for a gene therapy treatment adapted for systemic administration. In some cases, one or more regulatory elements of this disclosure are operably linked to a transgene for a gene therapy treatment adapted for administration to the central nervous system. In some cases, one or more regulatory elements of this disclosure are operably linked to a transgene for a gene therapy treatment adapted for administration to the cerebral spinal fluid. In some cases, one or more regulatory elements of this disclosure are operably linked to a transgene for a gene therapy treatment adapted for expression in neurons or glia.

### D. Vectors

In some embodiments, the disclosure provides for a vector (e.g., any of the vectors disclosed herein) comprising any of the nucleic acid molecules disclosed herein. In some embodiments, the vector is a viral vector (e.g., an adeno-associated viral vector). In some embodiments, the vector is a viral particle. In some embodiments, the vector is a non-viral vector. In some embodiments, any of the methods disclosed herein may be used to administer any of the vectors disclosed herein to a subject (e.g., a primate).

In some embodiments, the nucleic acid molecules described herein are provided (or delivered) to cells or tissue, in vitro or in vivo, using various known and suitable methods available in the art. In some embodiments, the nucleic acid molecules described herein are provided (or delivered) to cells or tissue, in vitro or in vivo, using methods described herein. Conventional viral and non-viral based gene delivery methods can be used to introduce the nucleic acid molecules disclosed herein into cells (e.g., neuronal cells) and target tissues. Non-viral expression vector systems include nucleic acid vectors such as, e.g., linear oligonucleotides and circular plasmids; artificial chromosomes such as human artificial chromosomes (HACs), yeast artificial chromosomes (YACs), and bacterial artificial chromosomes (BACs or PACs)); episomal vectors; transposons (e.g., PiggyBac); and cosmids. Viral vector delivery systems include DNA and RNA viruses, such as, e.g., retroviral vectors, lentiviral vectors, adenoviral vectors, and adeno-associated viral vectors. Methods of incorporating the nucleic acid molecules described herein into any of the non-viral and viral expression systems are known to those of skill in the art.

Methods and compositions for non-viral delivery of nucleic acids are known in the art, including physical and chemical methods. Physical methods generally refer to methods of delivery employing a physical force to counteract the cell membrane barrier in facilitating intracellular delivery of genetic material. Examples of physical methods include the use of a needle, ballistic DNA, electroporation, sonoporation, photoporation, magnetofection, and hydroporation. Chemical methods generally refer to methods in which chemical carriers deliver a nucleic acid molecule to a cell and may include inorganic particles, lipid-based carriers, polymer-based carriers and peptide-based carriers.

In some embodiments, a non-viral expression vector is administered to a target cell using an inorganic particle. Inorganic particles may refer to nanoparticles, such as nanoparticles that are engineered for various sizes, shapes, and/or porosity to escape from the reticuloendothelial system or to protect an entrapped molecule from degradation. Inorganic nanoparticles can be prepared from metals (e.g., iron, gold, and silver), inorganic salts, or ceramics (e.g., phosphate or carbonate salts of calcium, magnesium, or silicon). The surface of these nanoparticles can be coated to facilitate DNA binding or targeted gene delivery. Magnetic nanoparticles (e.g., supermagnetic iron oxide), fullerenes (e.g., soluble carbon molecules), carbon nanotubes (e.g., cylindrical fullerenes), quantum dots and supramolecular systems may also be used.

In some embodiments, a non-viral expression vector is administered to a target cell using a cationic lipid (e.g., cationic liposome). Various types of lipids have been investigated for gene delivery, such as, for example, a lipid nano-emulsion (e.g., which is a dispersion of one immiscible liquid in another stabilized by emulsifying agent) or a solid lipid nanoparticle. In some embodiments, a non-viral expression vector can be delivered using lipid nanoparticles (LNPs). In some embodiments, the LNPs comprise cationic lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., lipids of WO2017/173054, WO2015/095340, and WO2014/136086, as well as references provided therein.

In some embodiments, a non-viral expression vector is administered to a target cell using a peptide based delivery vehicle. Peptide based delivery vehicles can have advantages of protecting the genetic material to be delivered, targeting specific cell receptors, disrupting endosomal membranes and delivering genetic material into a nucleus. In some embodiments, a non-viral expression vector is administered to a target cell using a polymer based delivery vehicle. Polymer based delivery vehicles may comprise natural proteins, peptides and/or polysaccharides or synthetic polymers. In one embodiment, a polymer based delivery vehicle comprises polyethylenimine (PEI). PEI can condense DNA into positively charged particles which bind to anionic cell surface residues and are brought into the cell via endocytosis. In other embodiments, a polymer based delivery vehicle may comprise poly-L-lysine (PLL), poly (DL-lactic acid) (PLA), poly (DL-lactide-co-glycoside) (PLGA), polyornithine, polyarginine, histones, protamines, dendrimers, chitosans, synthetic amino derivatives of dextran, and/or cationic acrylic polymers. In certain embodiments, polymer based delivery vehicles may comprise a mixture of polymers, such as, for example PEG and PLL.

In some embodiments, any of the nucleic acid molecules disclosed herein can be delivered using any known suitable viral vector including, e.g., retroviruses (e.g., A-type, B-type, C-type, and D-type viruses), adenovirus, parvovirus (e.g. adeno-associated viruses or AAV), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Examples of retroviruses include avian leukosis-sarcoma virus, human T-lymphotrophic virus type 1 (HTLV-1), bovine leukemia virus (BLV), lentivirus, and spumavirus. Other viruses include Norwalk virus, togavirus, flavivirus, reoviruses, papovavirus, hepadnavirus, and hepatitis virus, for example. Viral vectors may be classified into two groups according to their ability to integrate into the host genome—integrating and non-integrating. Oncoretroviruses and lentiviruses can integrate into host cellular chromatin while adenoviruses, adeno-associated viruses, and herpes viruses predominantly persist in the cell nucleus as extrachromosomal episomes.

In some embodiments, a suitable viral vector is a retroviral vector. Retroviruses refer to viruses of the family Retroviridae. Examples of retroviruses include oncoretroviruses, such as murine leukemia virus (MLV), and lentiviruses, such as human immunodeficiency virus 1 (HIV-1). Retroviral genomes are single-stranded (ss) RNAs and comprise various genes that may be provided in cis or trans. For example, a retroviral genome may contain cis-acting sequences such as two long terminal repeats (LTR), with elements for gene expression, reverse transcription and integration into the host chromosomes. Other components include the packaging signal (psi or ψ), for the specific RNA packaging into newly formed virions and the polypurine tract (PPT), the site of the initiation of the positive strand DNA synthesis during reverse transcription. In addition, in some embodiments, the retroviral genome may comprise gag, pol and env genes. The gag gene encodes the structural proteins, the pol gene encodes the enzymes that accompany the ssRNA and carry out reverse transcription of the viral RNA to DNA, and the env gene encodes the viral envelope. Generally, the gag, pol and env are provided in trans for viral replication and packaging.

In some embodiments, a retroviral vector provided herein may be a lentiviral vector. At least five serogroups or serotypes of lentiviruses are recognized. Viruses of the different serotypes may differentially infect certain cell types and/or hosts. Lentiviruses, for example, include primate retroviruses and non-primate retroviruses. Primate retroviruses include HIV and simian immunodeficiency virus (SIV). Non-primate retroviruses include feline immunodeficiency virus (FIV), bovine immunodeficiency virus (BIV), caprine arthritis-encephalitis virus (CAEV), equine infectious anemia virus (EIAV) and visnavirus. Lentiviruses or lentivectors may be capable of transducing quiescent cells. As with oncoretrovirus vectors, the design of lentivectors may be based on the separation of cis- and trans-acting sequences.

In some embodiments, the present disclosure provides expression vectors that have been designed for delivery by an optimized therapeutic retroviral vector. The retroviral vector can be a lentivirus comprising any one or more of: a left (5′) LTR; sequences which aid packaging and/or nuclear import of the virus; a promoter; optionally one or more additional regulatory elements (such as, for example, an enhancer or polyA sequence); optionally a lentiviral reverse response element (RRE); optionally an insulator; and a right (3′) retroviral LTR.

In some embodiments, a viral vector provided herein is an adeno-associated virus (AAV). AAV is a small, replication-defective, non-enveloped animal virus that infects humans and some other primate species. AAV is not known to cause human disease and induces a mild immune response. AAV vectors can also infect both dividing and quiescent cells without integrating into the host cell genome.

The AAV genome naturally consists of a linear single stranded DNA which is ˜4.7 kb in length. The genome consists of two open reading frames (ORF) flanked by an inverted terminal repeat (ITR) sequence that is about 145 bp in length. The ITR consists of a nucleotide sequence at the 5′ end (5′ ITR) and a nucleotide sequence located at the 3′ end (3′ ITR) that contain palindromic sequences. The ITRs function in cis by folding over to form T-shaped hairpin structures by complementary base pairing that function as primers during initiation of DNA replication for second strand synthesis. The two open reading frames encode for rep and cap genes that are involved in replication and packaging of the virion. In some embodiments, an AAV vector provided herein does not contain the rep or cap genes. Such genes may be provided in trans for producing virions as described further below.

In some embodiments, an AAV vector may include a stuffer nucleic acid. In some embodiments, the stuffer nucleic acid may encode a green fluorescent protein or antibiotic resistance gene providing resistance to antibiotics such as kanamycin or ampicillin. In certain embodiments, the stuffer nucleic acid may be located outside of the ITR sequences (e.g., as compared to the transgene sequence and regulatory sequences, which are located between the 5′ and 3′ ITR sequences).

In some embodiments, the AAV vector is any one of AAV1, AAV2, AAV3, AAV3b, AAV4, AAV5, AAV6, AAV7, AAV8, AAV9, AAV10, AAV11, AAV12, AAV13, AAV-DJ, AAV-DJ8, AAV-DJ9 or a chimeric, hybrid, or variant AAV. The AAV can also be a self-complementary AAV (scAAV). These serotypes differ in their tropism, or the types of cells they infect. In some embodiments, the AAV vector comprises the genome and capsids from multiple serotypes (e.g., pseudotypes). For example, an AAV may comprise the genome of serotype 2 (e.g., ITRs) packaged in the capsid from serotype 5 or serotype 9. Pseudotypes may improve transduction efficiency as well as alter tropism. In some embodiments, the AAV is an AAV9 serotype. In certain embodiments, an expression vector designed for delivery by an AAV comprises a 5′ ITR and a 3′ ITR.

In some embodiments, the ITRs of AAV serotype 6 or AAV serotype 9 can be used in any of the AAV vectors disclosed herein. However, ITRs from other suitable serotypes may be selected. AAV vectors of the present disclosure may be generated from a variety of adeno-associated viruses. The tropism of the vector may be altered by packaging the recombinant genome of one serotype into capsids derived from another AAV serotype. In some embodiments, the ITRs of the rAAV virus can be based on the ITRs of any one of AAV1-12 and may be combined with an AAV capsid selected from any one of AAV1-12, AAV-DJ, AAV-DJ8, AAV-DJ9 or other modified serotypes. In particular embodiments, the AAV ITRs and/or capsids are selected based on the cell or tissue to be targeted with the AAV vector.

In some embodiments, the disclosure provides for a vector comprising any of the nucleic acids disclosed herein, wherein the vector is an AAV vector or an AAV viral particle, or virion. In some embodiments, an AAV vector or an AAV viral particle, or virion, can be used to deliver any of the nucleic acid molecules disclosed herein comprising any of the regulatory elements disclosed herein operably linked to any of the transgenes disclosed herein, either in vivo, ex vivo, or in vitro. In some embodiments, such an AAV vector is replication-deficient. In some embodiments, an AAV virus is engineered or genetically modified so that it can replicate and generate virions only in the presence of helper factors.

In some embodiments, an expression vector designed for delivery by an AAV comprises a 5′ ITR, a promoter, a nucleic acid molecule comprising a regulatory element operably linked to a transgene (e.g. a transgene encoding SMNA1), and a 3′ ITR. In some embodiments, an expression vector designed for delivery by an AAV comprises a 5′ ITR, an enhancer, a promoter, a nucleic acid molecule comprising a regulatory element operably linked to a transgene (e.g. a transgene encoding SMNA1), a polyA sequence, and a 3′ ITR.

In some embodiments, the present disclosure provides for a viral vector comprising any of the nucleic acids disclosed herein. The terms “viral particle”, and “virion” are used herein interchangeably and relate to an infectious and typically replication-defective virus particle comprising the viral genome (e.g., the viral expression vector) packaged within a capsid and, as the case may be e.g., for retroviruses, a lipidic envelope surrounding the capsid. A “capsid” refers to the structure in which the viral genome is packaged. A capsid consists of several oligomeric structural subunits made of proteins. For example, AAV have an icosahedral capsid formed by the interaction of three capsid proteins: VP1, VP2 and VP3. In some embodiments, a virion provided herein is a recombinant AAV virion obtained by packaging an AAV vector that comprises a candidate regulatory element operably linked to a transgene and barcode sequence, as described herein, in a protein shell.

In some embodiments, a recombinant AAV virion provided herein may be prepared by encapsidating an AAV genome derived from a particular AAV serotype in a viral particle formed by natural Cap proteins corresponding to an AAV of the same particular serotype. In other embodiments, an AAV viral particle provided herein comprises a viral vector comprising ITR(s) of a given AAV serotype packaged into proteins from a different serotype. See e.g., Bunning H et al. J Gene Med 2008; 10: 717-733. For example, a viral vector having ITRs from a given AAV serotype may be packaged into: a) a viral particle constituted of capsid proteins derived from a same or different AAV serotype (e.g. AAV2 ITRs and AAV9 capsid proteins; AAV2 ITRs and AAV8 capsid proteins; etc.); b) a mosaic viral particle constituted of a mixture of capsid proteins from different AAV serotypes or mutants (e.g. AAV2 ITRs with AAV1 and AAV9 capsid proteins); c) a chimeric viral particle constituted of capsid proteins that have been truncated by domain swapping between different AAV serotypes or variants (e.g. AAV2 ITRs with AAV8 capsid proteins with AAV9 domains); or d) a targeted viral particle engineered to display selective binding domains, enabling stringent interaction with target cell specific receptors (e.g. AAV5 ITRs with AAV9 capsid proteins genetically truncated by insertion of a peptide ligand; or AAV9 capsid proteins non-genetically modified by coupling of a peptide ligand to the capsid surface).

The skilled person will appreciate that an AAV virion provided herein may comprise capsid proteins of any AAV serotype. In one embodiment, the viral particle comprises capsid proteins from an AAV serotype selected from the group consisting of an AAV1, an AAV2, an AAV5, an AAV6, an AAV8, and an AAV9.

Numerous methods are known in the art for production of recombinant AAV (rAAV) virions, including transfection, stable cell line production, and infectious hybrid virus production systems which include adenovirus-AAV hybrids, herpesvirus-AAV hybrids (Conway, J E et al., (1997) J. Virology 71(11):8780-8789) and baculovirus-AAV hybrids. In some embodiments, rAAV production cultures for the production of rAAV virus particles comprise; 1) suitable host cells, including, for example, human-derived cell lines such as HeLa, A549, or 293 cells, or insect-derived cell lines such as SF-9, in the case of baculovirus production systems; 2) suitable helper virus function, provided by wild-type or mutant adenovirus (such as temperature sensitive adenovirus), herpes virus, baculovirus, or a plasmid construct providing helper functions; 3) AAV rep and cap genes and gene products; 4) a nucleic acid molecule comprising a candidate regulatory element operably linked to a transgene (e.g., a nucleotide sequence encoding a nuclear binding domain operably linked to a reporter gene sequence as described herein), flanked by AAV ITR sequences; wherein the nucleic acid molecule comprises one or more barcode sequences, and 5) suitable media and media components to support rAAV production.

In some embodiments, the producer cell line is an insect cell line (typically Sf9 cells) that is infected with baculovirus expression vectors that provide Rep and Cap proteins. This system does not require adenovirus helper genes (Ayuso E, et al., Curr. Gene Ther. 2010, 10:423-436).

The term “cap protein”, as used herein, refers to a polypeptide having at least one functional activity of a native AAV Cap protein (e.g. VP1, VP2, VP3). Examples of functional activities of cap proteins include the ability to induce formation of a capsid, facilitate accumulation of single-stranded DNA, facilitate AAV DNA packaging into capsids (i.e. encapsidation), bind to cellular receptors, and facilitate entry of the virion into host cells. In principle, any Cap protein can be used in the context of the present disclosure.

Cap proteins have been reported to have effects on host tropism, cell, tissue, or organ specificity, receptor usage, infection efficiency, and immunogenicity of AAV viruses. Accordingly, an AAV cap for use in an rAAV may be selected taking into consideration, for example, the subject's species (e.g. human or non-human), the subject's immunological state, the subject's suitability for long or short-term treatment, or a particular therapeutic application (e.g. treatment of a particular disease or disorder, or delivery to particular cells, tissues, or organs). In certain embodiments, the cap protein is derived from the AAV of the group consisting of AAV1, AAV2, AAV5, AAV6, AAV8, and AAV9 serotypes.

In some embodiments, an AAV Cap for use in the methods provided herein can be generated by mutagenesis (i.e., by insertions, deletions, or substitutions) of one of the aforementioned AAV caps or its encoding nucleic acid. In some embodiments, the AAV cap is at least 70%, 75%, 80%, 85%, 90%, 95%, 98%, or 99% or more similar to one or more of the aforementioned AAV caps.

In some embodiments, the AAV cap is chimeric, comprising domains from two, three, four, or more of the aforementioned AAV caps. In some embodiments, the AAV cap is a mosaic of VP1, VP2, and VP3 monomers originating from two or three different AAV or a recombinant AAV. In some embodiments, a rAAV composition comprises more than one of the aforementioned caps.

In some embodiments, an AAV cap for use in a rAAV virion is engineered to contain a heterologous sequence or other modification. For example, a peptide or protein sequence that confers selective targeting or immune evasion may be engineered into a cap protein. Alternatively or in addition, the cap may be chemically modified so that the surface of the rAAV is polyethylene glycolated (i.e., pegylated), which may facilitate immune evasion. The cap protein may also be mutagenized (e.g., to remove its natural receptor binding, or to mask an immunogenic epitope).

The term “rep protein”, as used herein, refers to a polypeptide having at least one functional activity of a native AAV rep protein (e.g., rep 40, 52, 68, 78). Examples of functional activities of a rep protein include any activity associated with the physiological function of the protein, including facilitating replication of DNA through recognition, binding and nicking of the AAV origin of DNA replication as well as DNA helicase activity. Additional functions include modulation of transcription from AAV (or other heterologous) promoters and site-specific integration of AAV DNA into a host chromosome. In some embodiments, AAV rep genes may be from the serotypes AAV1, AAV2, AAV4, AAV5, AAV6, AAV7, AAV8, AAV9, AAV10 or AAVrh10.

In some embodiments, an AAV rep protein for use in the method of the invention can be generated by mutagenesis (i.e. by insertions, deletions, or substitutions) of one of the aforementioned AAV reps or its encoding nucleic acid. In some embodiments, the AAV rep is at least 70%, 75%, 80%, 85%, 90%, 95%, 98%, or 99% or more similar to one or more of the aforementioned AAV reps.

The expressions “helper functions” or “helper genes”, as used herein, refer to viral proteins upon which AAV is dependent for replication. The helper functions include those proteins required for AAV replication including, without limitation, those proteins involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus. Helper functions include, without limitation, adenovirus E1, E2a, VA, and E4 or herpesvirus UL5, ULB, UL52, and UL29, and herpesvirus polymerase. In a preferred embodiment, the proteins upon which AAV is dependent for replication are derived from adenovirus.

In some embodiments, a viral protein upon which AAV is dependent for replication for use in the method of the invention can be generated by mutagenesis (i.e. by insertions, deletions, or substitutions) of one of the aforementioned viral proteins or its encoding nucleic acid. In some embodiments, the viral protein is at least 70%, 75%, 80%, 85%, 90%, 95%, 98%, or 99% or more similar to one or more of the aforementioned viral proteins.

Methods for assaying the functions of cap proteins, rep proteins and viral proteins upon which AAV is dependent for replication are well known in the art.

In some embodiments, a viral expression vector can be associated with a lipid delivery vehicle (e.g., cationic liposome or LNPs as described here) for administering to a target cell.

The various delivery systems containing the nucleic acid molecules described herein or known in the art can be administered to an organism for delivery to cells in vivo or administered to a cell or cell culture ex vivo. Administration is by any of the routes normally used for introducing a molecule into ultimate contact with blood, fluid, or cells including, but not limited to, injection, infusion, topical application and electroporation. Suitable methods of administering such nucleic acids are available and known to those of skill in the art.

The nucleic acid molecules can be delivered in vivo, or ex vivo to target various cells and/or tissues. In some embodiments, delivery can be targeted to various organs/tissues and corresponding cells, e.g., to the brain, heart, skeletal muscle, liver, kidney, spleen, or stomach. In some embodiments, the nucleic acid molecules are delivered to one or both of neuronal cells or glial cells. In some embodiments, delivery can be targeted to diseased cells, such as, e.g., tumor or cancer cells. In some embodiments, delivery can be targeted to stem cells, blood cells, or immune cells.

In some embodiments, the disclosure provides for a mixture of any of the vectors disclosed herein, or any of the nucleic acids disclosed herein. In some embodiments, the mixture or nucleic acid molecules comprises about 10, about 50, about 100, about 250, about 500, about 750, about 1000, about 1250, about 1500, about 1750, about 2000, about 2500, about 3000, about 3500, about 4000, about 4500, about 5000, about 5500, about 6000, about 6500, about 7000, about 7500, about 8000, about 8500, about 9000, about 9500, about 10000, or more different regulatory elements.

### E. Pharmaceutical Compositions

In certain embodiments, the disclosure provides compositions comprising any of the nucleic acid constructs, expression vectors, viral vectors, or viral particles disclosed herein. In some embodiments, the disclosure provides compositions comprising a viral vector or viral particle which comprises a nucleotide sequence operably linked to a regulatory element. In particular embodiments, such compositions are suitable for gene therapy applications. Pharmaceutical compositions are preferably sterile and stable under conditions of manufacture and storage. Sterile solutions may be accomplished, for example, by filtration through sterile filtration membranes.

Acceptable carriers and excipients in the pharmaceutical compositions are preferably nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, and lysine, and carbohydrates such as glucose, mannose, sucrose, and sorbitol. Pharmaceutical compositions of the disclosure can be administered parenterally in the form of an injectable formulation. Pharmaceutical compositions for injection can be formulated using a sterile solution or any pharmaceutically acceptable liquid as a vehicle. Pharmaceutically acceptable vehicles include, but are not limited to, sterile water and physiological saline.

The pharmaceutical compositions of the disclosure may be prepared in microcapsules, such as hydroxylmethylcellulose or gelatin-microcapsules and polymethylmethacrylate microcapsules. The pharmaceutical compositions of the disclosure may also be prepared in other drug delivery systems such as liposomes, albumin microspheres, microemulsions, nanoparticles, and nanocapsules. The pharmaceutical composition for gene therapy can be in an acceptable diluent, or can comprise a slow release matrix in which the gene delivery vehicle is embedded.

Pharmaceutical compositions provided herein may be formulated for parenteral administration, subcutaneous administration, intravenous administration, systemic administration, intramuscular administration, intra-arterial administration, intraparenchymal administration, intrathecal administration, intrathecal cisternal administration (also known as intra-cisterna magna administration), intrathecal lumbar administration, intracerebroventricular administration, or intraperitoneal administration. In a particular embodiment, the pharmaceutical composition is formulated for intracerebroventricular administration. In one embodiment, the pharmaceutical composition is formulated for intrathecal administration. In one embodiment, the pharmaceutical composition is formulated for intrathecal cisternal administration. In one embodiment, the pharmaceutical composition is formulated for intrathecal lumbar administration. In one embodiment, the pharmaceutical composition is formulated for intravenous administration. In one embodiment, the pharmaceutical composition is formulated for systemic administration.

The pharmaceutical composition may be formulated for, or administered via nasal, spray, oral, aerosol, rectal, or vaginal administration. The tissue target may be specific, for example the central nervous system, or it may be a combination of several tissues, for example the central nervous system and liver tissues. Exemplary tissue or other targets may include liver, skeletal muscle, heart muscle, adipose deposits, kidney, lung, vascular endothelium, epithelial, hematopoietic cells, neuronal cells, glial cells, central nervous system and/or CSF. In a particular embodiment, a pharmaceutical composition provided herein is administered to the CSF, i.e. by intracerebroventricular injection, intrathecal cisternal injection or intrathecal lumbar injection. One or more of these methods may be used to administer a pharmaceutical composition of the disclosure.

In certain embodiments, a pharmaceutical composition provided herein comprises an “effective amount” or a “therapeutically effective amount.” As used herein, such amounts refer to an amount effective, at dosages and for periods of time necessary to achieve the desired therapeutic result.

The dosage of the pharmaceutical compositions of the disclosure depends on factors including the route of administration, the disease to be treated, and physical characteristics (e.g., age, weight, general health) of the subject. Dosage may be adjusted to provide the optimum therapeutic response. Typically, a dosage may be an amount that effectively treats the disease without inducing significant toxicity. In one embodiment, an AAV vector provided herein can be administered to the patient for the treatment of a neuronal disease (including for example, Dravet syndrome) in an amount or dose within a range of 5×10¹⁰ to 1×10¹⁴ gc/kg (genome copies per kilogram of patient body weight (gc/kg)). In a more particular embodiment, the AAV vector is administered in an amount comprised within a range of about 5×10¹⁰ gc/kg to about 1×10¹³ gc/kg, or about 1×10¹¹ to about 1×10¹⁵ gc/kg, or about 1×10¹¹ to about 1×10¹⁴ gc/kg, or about 1×10¹¹ to about 1×10¹³ gc/kg, or about 1×10¹¹ to about 1×10¹² gc/kg, or about 1×10¹² to about 1×10¹⁴ gc/kg, or about 1×10¹² to about 1×10¹³ gc/kg, or about 5×10¹¹ gc/kg, 1×10¹² gc/kg, 1.5×10¹² gc/kg, 2.0×10¹² gc/kg, 2.5×10¹² gc/kg, 3×10¹² gc/kg, 3.5×10¹² gc/kg, 4×10¹² gc/kg, 4.5×10¹² gc/kg, 5×10¹² gc/kg, 5.5×10¹² gc/kg, 6×10¹² gc/kg, 6.5×10¹² gc/kg, 7×10¹² gc/kg, 7.5×10¹² gc/kg, 8×10¹² gc/kg, 8.5×10¹² gc/kg, 9×10¹² gc/kg, 9.5×10¹² gc/kg, 1×10¹³ gc/kg, 1.5×10¹³ gc/kg, 2.0×10¹³ gc/kg, 2.5×10¹³ gc/kg, 3×10¹³ gc/kg, 3.5×10¹³ gc/kg, 4×10¹³ gc/kg, 4.5×10¹³ gc/kg, 5×10¹³ gc/kg, 5.5×10¹³ gc/kg, 6×10¹³ gc/kg, 6.5×10¹³ gc/kg, 7×10¹³ gc/kg, 7.5×10¹³ gc/kg, 8×10¹³ gc/kg, 8.5×10¹³ gc/kg, 9×10¹³ gc/kg, or 9.5×10¹³ gc/kg. The gc/kg may be determined, for example, by qPCR or digital droplet PCR (ddPCR) (see e.g., M. Lock et al, Hum Gene Ther Methods. 2014 April; 25(2): 115-25). In another embodiment, an AAV vector provided herein can be administered to the patient for the treatment of a neuronal disease (including for example, Dravet syndrome) in an amount or dose within a range of 1×10⁹ to 1×10¹¹ iu/kg (infective units of the vector (iu)/subject's or patient's body weight (kg)). In certain embodiments, the pharmaceutical composition may be formed in a unit dose as needed. Such single dosage units may contain about 1×10⁹ gc to about 1×10¹⁵ gc.

Pharmaceutical compositions of the disclosure may be administered to a subject in need thereof, for example, one or more times (e.g., 1-10 times or more) daily, weekly, monthly, biannually, annually, or as medically necessary. In an exemplary embodiment, a single administration is sufficient. In one embodiment, the pharmaceutical composition is suitable for use in human subjects and is administered by intracerebroventricular administration. In one embodiment, the pharmaceutical composition is suitable for use in human subjects and is administered by intracerebroventricular administration, intravenous administration, intrathecal administration, intraparenchymal administration, or combinations thereof. In one embodiment, the pharmaceutical composition is delivered via a peripheral vein by bolus injection. In other embodiments, the pharmaceutical composition is delivered via a peripheral vein by infusion over about 10 minutes (±5 minutes), over about 20 minutes (±5 minutes), over about 30 minutes (±5 minutes), over about 60 minutes (±5 minutes), or over about 90 minutes (±10 minutes). In one embodiment, the pharmaceutical composition is delivered to the CSF by bolus injection. In other embodiments, the pharmaceutical composition is delivered to the CSF by infusion over about 10 minutes (±5 minutes), over about 20 minutes (±5 minutes), over about 30 minutes (±5 minutes), over about 60 minutes (±5 minutes), or over about 90 minutes (±10 minutes).

In another aspect, the disclosure further provides a kit comprising a nucleic acid construct, viral vector, viral particle, or pharmaceutical composition as described herein in one or more containers. A kit may include instructions or packaging materials that describe how to administer a nucleic acid molecule, vector, or virion contained within the kit to a patient. Containers of the kit can be of any suitable material, e.g., glass, plastic, metal, etc., and of any suitable size, shape, or configuration. In certain embodiments, the kits may include one or more ampoules or syringes that contain a nucleic acid construct, viral vector, viral particle, or pharmaceutical composition in a suitable liquid or solution form.

### F. Methods of Administration

In some embodiments, the disclosure provides for methods of administering any of the nucleic acid constructs, viral vectors, viral particles, and/or pharmaceutical compositions disclosed herein to a subject (e.g., a primate) in need thereof via any of the routes of administration disclosed herein. In some embodiments, the method comprises administering any of the nucleic acid constructs, viral vectors, viral particles, and/or pharmaceutical compositions disclosed herein via intracerebroventricular administration. In some embodiments, the method comprises administering any of the nucleic acid constructs, viral vectors, viral particles, and/or pharmaceutical compositions disclosed herein via intravenous administration. In some embodiments, the method comprises administering any of the nucleic acid constructs, viral vectors, viral particles, and/or pharmaceutical compositions disclosed herein via intrathecal administration. In some embodiments, the method comprises administering any of the nucleic acid constructs, viral vectors, viral particles, and/or pharmaceutical compositions disclosed herein via intraparenchymal administration. Methods of administering any of the vectors disclosed herein are discussed in greater detail below. These methods could also be used for administering any of the nucleic acid constructs, viral particles, and/or pharmaceutical compositions disclosed herein.

The present disclosure contemplates methods of administering a vector to a primate (e.g., a human), comprising intracerebroventricular (ICV) administration of the vector. Also described herein are compositions and methods for expressing a gene of interest or a biologically active variant and/or fragment thereof comprising administering to a primate a therapeutically effective amount of an adeno-associated virus 1 (AAV1) vector or an adeno-associated virus 5 (AAV5) vector encoding the gene of interest, wherein the route of administration is selected from the group consisting of intravenous administration, intrathecal administration, intracerebroventricular administration, intraparenchymal administration, or combinations thereof. Furthermore, described herein are compositions and methods to inhibit or treat one or more symptoms associated with a neuronal disease in a primate in need thereof, comprising administering an AAV selected from the group consisting of AAV1 or AAV5 to the primate, wherein the route of administration is selected from the group consisting of intravenous administration, intrathecal administration, intracerebroventricular administration, intraparenchymal administration, or combinations thereof.

In some embodiments, the disclosure provides for methods of administering any of the vectors disclosed herein to a subject (e.g., a primate) via intrathecal administration or intracerebroventricular administration. The intrathecal space, into which the vector of the present invention is delivered in the case of intrathecal administration, is a space which is located around the spinal cord and filled with cerebrospinal fluid. This space is surrounded by a double-layer membrane consisting of arachnoid mater and dura mater. The intrathecal space is a space beneath the arachnoid mater, the inner layer of the double-layer membrane, and therefore, intrathecal administration means administration into the subarachnoid space. The space around the brain and the space around the spinal cord are both filled with CSF, and the cerebral ventricles in the brain are also filled with CSF. The cerebral ventricles, the pericerebral space and the intrathecal space are connected to form one continuous space, in which the CSF circulates. Therefore, intracerebroventricular administration and intrathecal administration are contemplated as being methods of administering any of the vectors disclosed herein to the CSF.

In some embodiments, the disclosure provides for methods of administering any of the vectors disclosed herein to a subject (e.g., a primate). In some embodiments, the vector is delivered to the CNS. In some embodiments, the vector is delivered to the cerebrospinal fluid. In some embodiments, the vector is administered to the brain parenchyma. In some embodiments, the vector is delivered to a primate by intracerebroventricular administration. In some embodiments, the vector is delivered to a subject (e.g., a primate) by intravenous administration. In some embodiments, the vector is delivered to a subject (e.g., a primate) by intrathecal administration, e.g. intrathecal cisternal or intrathecal lumbar administration. In some embodiments, the vector is delivered to the subarachnoid cistern, e.g. the cisterna magna. In some embodiments, the vector is delivered into the lumbar subarachnoid space surrounding the spinal nerves. In some embodiments, the vector is delivered to a subject (e.g., a primate) by intraparenchymal administration. Broad distribution of vectors, described herein, within the central nervous system may be achieved with intraparenchymal administration, intrathecal administration, or intracerebroventricular administration.

In some embodiments, any of the vectors disclosed herein is administered to a subject (e.g., a primate) in combination with a contrast agent, e.g. gadolinium or gadoteridol. In other embodiments, the vector is not administered in combination with a contrast agent, e.g. gadolinium or gadoteridol.

In some embodiments, any of the vectors disclosed herein is administered via intracerebroventricular (ICV) administration to any one or more ventricles of the brain. In some embodiments, the vector is administered via ICV administration unilaterally into one ventricle, e.g. into the left lateral ventricle or right lateral ventricle. In some embodiments, the vector is administered via ICV administration unilaterally into the left lateral ventricle. In some embodiments, the vector is administered via ICV administration unilaterally into the right lateral ventricle. In some embodiments, the vector is administered via ICV administration bilaterally, e.g. into the left and right lateral ventricle. In some embodiments, the vector is administered via ICV administration to one ventricle of the brain, e.g. into only the left ventricle. In some embodiments, the vector is administered via ICV administration to only the left lateral ventricle. In some embodiments, the vector is administered via ICV administration to only the right lateral ventricle. In some embodiments, the vector is administered via ICV administration to only the third ventricle. In some embodiments, the vector is administered via ICV administration to only the fourth ventricle. In some embodiments, the vector is administered via ICV administration to more than one ventricle of the brain, e.g. into the left ventricle, right ventricle, and third ventricle. In some embodiments, the vector is administered via ICV administration simultaneously, e.g., into the left ventricle and right ventricle at the same time point. In some embodiments, the vector is administered via ICV administration sequentially, e.g. into the left ventricle and right ventricle at different time points. In some embodiments, each dose of the vector is administered via ICV administration at least 24 hours apart.

In some embodiments, the disclosure provides a method of administering a vector to a primate, comprising intracerebroventricular (ICV) administration of a vector to the primate, wherein the vector comprises a transgene, and wherein ICV administration results in increased transgene expression in the central nervous system (CNS) by at least 1.25-fold as compared to expression of the transgene when the vector is administered by any other route of administration. In certain embodiments, ICV administration produces at least 1.5-fold, 1.75-fold, 2-fold, 3-fold 5-fold, 10-fold, 15-fold, 20-fold, 25-fold, 30-fold, 35-fold, 40-fold, 45-fold, 50-fold, 55-fold, 60-fold, 65-fold, 70-fold, or 75-fold, or at least 20-90 fold, 20-80 fold, 20-70 fold, 20-60 fold, 30-90 fold, 30-80 fold, 30-70 fold, 30-60 fold, 40-90 fold, 40-80 fold, 40-70 fold, 40-60 fold, 50-90 fold, 50-80 fold, 50-70 fold, 50-60 fold, 60-90 fold, 60-80 fold, 60-70 fold, 70-90 fold, 70-80 fold, 80-90 fold greater expression of the transgene sequence in the central nervous system (CNS) as compared to expression of the transgene when the vector is administered by any other route of administration. In some embodiments, ICV administration results in gene transfer throughout the brain. In certain embodiments, the gene transfer occurs in the frontal cortex, parietal cortex, temporal cortex, hippocampus, medulla, and occipital cortex. In certain embodiments, the gene transfer is dose dependent. In certain embodiments, the vector further comprises a cell-type selective regulatory element. In certain embodiments, the regulatory element is selectively expressed in the brain. In certain embodiments, the regulatory element is selectively expressed in the frontal cortex, parietal cortex, temporal cortex, hippocampus, medulla, and occipital cortex. In certain embodiments, the regulatory element is selectively expressed in the spine. In certain embodiments, the regulatory element is selectively expressed in the spinal cord and dorsal root ganglion. In certain embodiments, the regulatory element is selectively expressed in neuronal cells. In certain embodiments, the neuronal cells are selected from the group consisting of unipolar, bipolar, multipolar, or pseudounipolar neurons. In certain embodiments, the neuronal cells are GABAergic neurons. In certain embodiments, the regulatory element is selectively expressed in glial cells. In certain embodiments, the glial cells are selected from the group consisting of astrocytes, oligodendrocytes, ependymal cells, Schwann cells, and satellite cells. In certain embodiments, the regulatory element is selectively expressed in non-neuronal cells.

In some embodiments, the disclosure provides for administering any of the vectors disclosed herein by multiple routes of administration to a subject (e.g., a primate). In some embodiments, the disclosure provides for methods of administering any of the vectors disclosed herein by one route of administration (e.g., intracerebroventricular administration) and the same vector(s) also by another route of administration (e.g., intravenous administration). In some embodiments, the disclosure provides for methods of administering any of the vectors disclosed herein by intracerebroventricular administration and the same vector(s) also by intravenous administration. In some embodiments, the disclosure provides for methods of administering any of the vectors disclosed herein by intrathecal administration and the same vector(s) also by intravenous administration. In some embodiments, the disclosure provides for methods of administering any of the vectors disclosed herein by one route of administration (e.g., intracerebroventricular administration) and an additional therapeutic agent (e.g., any of the additional therapeutic agents disclosed herein) by another route of administration (e.g., intravenous administration). In some embodiments, the disclosure provides for methods of administering any of the vectors disclosed herein by intracerebroventricular administration and an additional therapeutic agent by intravenous administration. In some embodiments, the disclosure provides for methods of administering any of the vectors disclosed herein by intrathecal administration and an additional therapeutic agent by intravenous administration. In some embodiments, the disclosure provides for methods of administering any of the vectors disclosed herein by intravenous administration and an additional therapeutic agent by intracerebroventricular administration. In some embodiments, the disclosure provides for methods of administering any of the vectors disclosed herein by intravenous administration and an additional therapeutic agent by intrathecal administration. In some embodiments, the intrathecal administration comprises an intrathecal cisternal administration. In some embodiments, the intrathecal administration comprises an intrathecal lumbar administration. In some embodiments, the route of administration is any one or combination of intravenous administration, intrathecal administration, intracerebroventricular administration, or intraparenchymal administration. In some embodiments, the route of administration is any one or combination of subcutaneous administration, intramuscular administration, intraarterial administration, intraperitoneal administration, or intracranial administration.

In some embodiments, the administration comprises administration through an injection. In some embodiments, the administration comprises administration through a cannula. In some embodiments, the vector is administered as a bolus, e.g., as a single injection. In some embodiments, the vector is administered continuously, e.g., an infusion using a syringe pump.

In some embodiments, intracerebroventricular (ICV) administration comprises inserting a cannula through a hole in the skull, through the brain tissue, into a CSF-filled ventricle of the brain. In some embodiments, a single cannula is inserted (e.g. into either of the two lateral ventricles). In some embodiments, two cannulas may be inserted (into both lateral ventricles). In some embodiments, the cannula may be connected to a syringe or infusion pump for one-time administration, or a controlled device, such as an Ommaya reservoir. In some embodiments, the disclosure provides for administration of any of the vectors disclosed herein to one or more lateral ventricles of a subject. Because of the concern for neurovascular injury and intracranial hemorrhage, repeated “taps” of the ventricle are not routinely performed. An exception to this rule might be in premature neonates who during pathologic conditions often have very large ventricles, a thin cortical mantle, and an open fontanelle, making the cumulative risks of repeated taps lower in this population.

Intrathecal intracisternal infusions are less frequently performed in humans due to the proximity of the cisterns to vital brain tissues. However, in some embodiments, intrathecal infusion devices (e.g. Medtronic devices) can be inserted in the lumbar subarachnoid space and a catheter extended upwards toward the cranium for administration. In some embodiments, intrathecal administration to a human being comprises surgically inserting a catheter at about the L4/L5 interspace and administering either (i) a bolus dose (via syringe or Ommaya reservoir), (ii) a short term infusion (via a pump), or (iii) a long term infusion (via an implantable programmable pump system, e.g. Synchromed II, Medtronic, where the pump is placed in a subcutaneous pocket somewhere in the body such as the abdominal region). See, e.g., Hamza M, et al. Neuromodulation, 2015; 18(7):636-48).

In some embodiments, intrathecal administration of any of the vectors disclosed herein comprises administering the vector(s) into the lumbar cistern by means of a lumbar puncture. In some embodiments, a spinal tap can be performed at the bedside with local anesthetic under sterile conditions. In some embodiments, a spinal needle is advanced into the thecal sac through an interlaminar space in the lower lumbar spine. In some embodiments, access into the lumbar cistern is confirmed when CSF is obtained. See, e.g., Cook A M, et al. Pharmacotherapy. 2009; 29(7):832-45.

In some embodiments, any of the vectors disclosed herein are administered to a subject (e.g., a primate) by injecting the vector(s) through a spinal needle. This technique is used frequently for administration of chemotherapeutic drugs. Advantages of this technique include its relatively low risk and ability to be performed at the bedside under local anesthetic. The major disadvantage of this technique is that a separate puncture must be performed each time a dose is given, resulting in a cumulative risk of introducing infection, developing a cutaneous-CSF fistula, injuring nerve roots, and causing intraspinal hemorrhage. In some embodiments, to circumvent this problem, a temporary indwelling catheter can be placed by using a similar technique with a larger Touhy needle.

In some embodiments, any of the vectors disclosed herein may be administered to a subject (e.g., a primate) by advancing a catheter into the thecal sac of the subject through the center of the needle, wherein the needle is subsequently withdrawn. In some embodiments, the catheter is then tunneled subcutaneously through the skin where it can be accessed sterilely for scheduled doses of a chosen intrathecal drug. The main disadvantage of this technique include the risk of infection with prolonged catheter placement and catheter malfunction from occlusion, kinking, or displacement. However, this disadvantage may be mitigated by removing or replacing the catheter after a few days (e.g., 1-4 days).

In some embodiments, any of the vectors disclosed herein is administered via a catheter-based device. In some embodiments, a permanent catheter-based device is implanted. In some embodiments, a temporary catheter-based device is implanted. In some embodiments, for permanent access, a catheter that is connected to a subcutaneous reservoir (e.g., an Ommaya reservoir) is implanted. In some embodiments, the catheter is connected to the Ommaya reservoir. The Ommaya reservoir can be accessed repeatedly at the bedside with a sterile puncture through the scalp into the reservoir by using a 25-gauge needle. In some embodiments, a few milliliters of CSF is withdrawn before injecting the therapeutic agent. Contamination and infection of the Ommaya reservoir is a risk, although less likely than with other methods of accessing the intraventricular compartment (approximately 10% of patients ultimately have CSF contaminated with bacteria). Infection rates often appear higher in case series reporting infectious complications with Ommaya reservoirs because of the duration of implantation (often >1 yr) compared with other more temporary access devices. Other rare complications that may occur with Ommaya reservoirs include leukoencephalopathy, white matter necrosis, and intracerebral hemorrhage.

In situations that require limited access to the CSF space, a ventriculostomy can be placed. With this technique, the catheter is tunneled under the skin away from the burr hole. The catheter is usually connected to a sterile collection chamber. The catheter can be accessed sterilely as needed for administration of any of the vectors disclosed herein. In some embodiments, the vector may be administered by injecting the solution into the most proximal port of the ventriculostomy and flushing the solution into the brain with a small amount of normal saline (3-5 ml). After this instillation, the ventriculostomy tubing is typically clamped for at least 15 minutes to allow for the injected solution to equilibrate in the CSF before reopening the drain. Patients with persistently elevated intracranial pressure may not tolerate the abrupt cessation of CSF drainage, so ventriculostomy clamping should be done with caution and close monitoring of the patient. A ventriculostomy is ideal for a condition that requires a limited time period for CSF drainage or intraventricular administration of any of the vectors disclosed herein.

In some embodiments, the disclosure provides for methods of administering any of the vectors disclosed herein to a subject, wherein the subject is a primate. In some embodiments, the primate is a human. In some embodiments, the primate is a non-human primate. In some embodiments, the non-human primate is an old world monkey, an orangutan, a gorilla, a chimpanzee, a crab-eating macaque, a rhesus macaque or a pig-tailed macaque.

### G. Methods of Treatment

The present disclosure contemplates methods of treating a subject (e.g., a primate such as a human or a cynomolgus monkey) in need thereof, comprising administering to the subject any of the nucleic acids, vectors, viral particles, and/or compositions disclosed herein.

In some embodiments, the disclosure provides for methods of treating a primate (e.g., a human or a cynomolgus monkey) comprising intracerebroventricular (ICV) administration of any of the vectors disclosed herein to a primate. In particular embodiments, the disclosure provides compositions and methods for expressing a gene of interest or a biologically active variant and/or fragment thereof comprising administering to a primate (e.g., a human or cynomolgus monkey) in need thereof a therapeutically effective amount of an adeno-associated virus 1 (AAV1) vector and/or an adeno-associated virus 5 (AAV5) vector encoding a gene of interest. In some embodiments, the AAV1 or AAV5 vector is administered to the primate via intravenous administration, intrathecal administration, intracerebroventricular administration, intraparenchymal administration, or combinations thereof. The disclosure further provides for compositions and methods to inhibit or treat one or more symptoms associated with a neuronal disease or disorder in a primate (e.g., a human or cynomolgus monkey) in need thereof, comprising administering an adeno-associated vector (AAV) selected from the group consisting of adeno-associated vector 1 (AAV1) or adeno-associated vector 5 (AAV5) to said primate. In some embodiments, the AAV1 or AAV5 vector is administered to the primate via intravenous administration, intrathecal administration, intracerebroventricular administration, intraparenchymal administration, or combinations thereof.

In some embodiments, the disclosure provides methods for treating neuronal diseases or disorders. Neuronal diseases or disorders appropriate for treatment include, but are not limited to, Dravet Syndrome, Alzheimer's disease, Parkinson's disease, Huntington's disease, amyotrophic lateral sclerosis (ALS), spinal muscular atrophy (SMA), epilepsy, neurodegenerative disorders, motor disorders, movement disorders, mood disorders, motor neuron diseases, progressive muscular atrophy (PMA), progressive bulbar palsy, pseudobulbar palsy, primary lateral sclerosis, neurological consequences of AIDS, developmental disorders, multiple sclerosis, neurogenetic disorders, stroke, spinal cord injury and traumatic brain injury.

In certain embodiments, the disclosure provides methods for treating a neuronal disease or disorder in a subject (e.g., a primate) in need thereof comprising administering to the subject a therapeutically effective amount of any of the nucleic acid constructs, viral vectors, viral particles, and/or pharmaceutical compositions disclosed herein. In some embodiments, such subject has been diagnosed with or is at risk for a neuronal disease or disorder, wherein the neuronal disease or disorder is any one or more of: Dravet Syndrome, Alzheimer's disease, Parkinson's disease, Huntington's disease, amyotrophic lateral sclerosis (ALS), spinal muscular atrophy (SMA), epilepsy, neurodegenerative disorders, motor disorders, movement disorders, mood disorders, motor neuron diseases, progressive muscular atrophy (PMA), progressive bulbar palsy, pseudobulbar palsy, primary lateral sclerosis, neurological consequences of AIDS, developmental disorders, multiple sclerosis, neurogenetic disorders, stroke, spinal cord injury and traumatic brain injury.

In some cases, treatment using a nucleic acid construct, vector, viral vector, viral particle, or pharmaceutical composition described herein results in improved symptoms associated with a neuronal disease or disorder. For instance, a Parkinson's patient can be monitored symptomatically for improved motor functions indicating positive response to treatment. Administration of a therapy using a method as described herein to a subject at risk of developing a neuronal disorder can prevent the development of or slow the progression of one or more symptoms.

In certain embodiments, methods and compositions of this disclosure can be used to treat a subject who has been diagnosed with a neuronal disease, for example, Dravet syndrome. In various embodiments, any of the neuronal diseases or disorders disclosed herein are caused by a known genetic event (e.g., any of the SCN1A mutations known in the art) or have an unknown cause.

In certain embodiments, methods and compositions of this disclosure can be used to treat a subject who is at risk of developing a disease or disorder. In some embodiments, the subject can be known to be predisposed to a disease, for example, a neuronal disease (e.g. Dravet syndrome). In some embodiments, the subject can be predisposed to a disease due to a genetic event, or due to known risk factors. For example, a subject can carry a mutation in SCN1A which is associated with Dravet syndrome.

In certain embodiments, one or more additional therapeutic agents (e.g. pharmaceutical compounds) are co-administered with any of the nucleic acid constructs, viral vectors, viral particles, and/or pharmaceutical compositions disclosed herein. In certain embodiments, the additional therapeutic agent(s) are designed to treat the same disease, disorder, or condition as any of the nucleic acid constructs, viral vectors, viral particles, and/or pharmaceutical compositions disclosed herein. In certain embodiments, the additional therapeutic agent(s) is/are designed to treat a different disease, disorder, or condition as any of the nucleic acid constructs, viral vectors, viral particles, and/or pharmaceutical compositions disclosed herein. In certain embodiments, the additional therapeutic agent(s) is/are designed to treat an undesired side effect of one or more of any of the nucleic acid constructs, viral vectors, viral particles, and/or pharmaceutical compositions disclosed herein. In certain embodiments, any of the nucleic acid constructs, viral vectors, viral particles, and/or pharmaceutical compositions disclosed herein are administered in combination with an additional pharmaceutical agent to treat an undesired effect of the additional pharmaceutical agent. In certain embodiments, one or more therapeutic agents are co-administered with any of the nucleic acid constructs, viral vectors, viral particles, and/or pharmaceutical compositions disclosed herein to produce a combinational effect. In certain embodiments, one or more therapeutic agents are co-administered with any of the nucleic acid constructs, viral vectors, viral particles, and/or pharmaceutical compositions disclosed herein to produce a synergistic effect in the treated subject (e.g., primate).

In certain embodiments, any of the nucleic acid constructs, viral vectors, viral particles, and/or pharmaceutical compositions disclosed herein and an additional therapeutic agent are administered at the same time. In certain embodiments, any of the nucleic acid constructs, viral vectors, viral particles, and/or pharmaceutical compositions disclosed herein and an additional therapeutic agent are administered at different times. In certain embodiments, any of the nucleic acid constructs, viral vectors, viral particles, and/or pharmaceutical compositions disclosed herein and an additional therapeutic agent are prepared together in a single formulation. In certain embodiments, any of the nucleic acid constructs, viral vectors, viral particles, and/or pharmaceutical compositions disclosed herein and an additional therapeutic agent are prepared separately.

In certain embodiments, therapeutic agents that may be co-administered with any of the nucleic acid constructs, viral vectors, viral particles, and/or pharmaceutical compositions disclosed herein include antipsychotic agents, such as, e.g., haloperidol, chlorpromazine, clozapine, quetapine, and olanzapine; antidepressant agents, such as, e.g., fluoxetine, sertraline hydrochloride, venlafaxine and nortriptyline; tranquilizing agents such as, e.g., benzodiazepines, clonazepam, paroxetine, venlafaxin, and beta-blockers; mood-stabilizing agents such as, e.g., lithium, valproate, lamotrigine, and carbamazepine; paralytic agents such as, e.g., Botulinum toxin; and/or other experimental agents including, but not limited to, tetrabenazine (Xenazine), creatine, conezyme Q10, trehalose, docosahexanoic acids, ACR16, ethyl-EPA, atomoxetine, citalopram, dimebon, memantine, sodium phenylbutyrate, ramelteon, ursodiol, zyprexa, xenasine, tiapride, riluzole, amantadine, [123I]MNI-420, atomoxetine, tetrabenazine, digoxin, detromethorphan, warfarin, alprozam, ketoconazole, omeprazole, cholinesterase inhibitors, donepezil, rivastigmine, galantamine, levodopa, and minocycline.

In certain embodiments, one or more nucleic acid constructs, viral vectors, viral particles, and/or pharmaceutical compositions disclosed herein are administered in combination with an osmolyte, e.g. mannitol or sorbitol. In some embodiments, the osmolyte is a polyol/polyhydric alcohol, e.g. mannitol and sorbitol. In some embodiments, the osmolyte is a sugar, e.g., sucrose or maltose. In some embodiments, the osmolyte is an amino acid or its derivative, e.g. glycine or proline. In certain embodiments, the osmolyte is co-administered to the CSF by way of injection or infusion. In some embodiments, the osmolyte is introduced by intravascular injection or infusion, intracerebroventricular injection or infusion, intrathecal cisternal injection or infusion, or intrathecal lumbar injection or infusion. In some embodiments, the introduction of the osmolyte can be simultaneous with the administration of any of the nucleic acid constructs, viral vectors, viral particles, and/or pharmaceutical compositions disclosed herein. In some embodiments, the osmolyte can be introduced into the CSF before administration of any of the nucleic acid constructs, viral vectors, viral particles, and/or pharmaceutical compositions disclosed herein. In some embodiments, the osmolyte can be introduced into the CSF after administration of any of the nucleic acid constructs, viral vectors, viral particles, and/or pharmaceutical compositions disclosed herein.

In some embodiments, once the osmolyte (e.g., mannitol) and therapeutic agent (e.g., any of the nucleic acid constructs, viral vectors, viral particles, and/or pharmaceutical compositions disclosed herein) are prepared as a solution for administration to a subject, it is administered into the CSF. In some embodiments, the prepared solution is administered by the routes such as intravascular injection or infusion, intracerebroventricular injection or infusion, intrathecal cisternal injection or infusion, or intrathecal lumbar injection or infusion. In some embodiments, the injections or infusions are for a period of time and a flow rate appropriate for the specific nucleic acid construct, viral vector, viral particle, and/or pharmaceutical composition. In some embodiments, it may be more desirable to pre-infuse an osmolyte (e.g., mannitol) solution intrathecally so that it can act on the local environment before therapeutic is administered intrathecally.

### H. Examples

Gene therapy using adeno associated viral (AAV) vectors has transformational potential to treat disorders affecting the central nervous system. Studies in small animal models have shown that delivery of AAV vectors into the cerebrospinal fluid (CSF) can successfully result in gene transfer to cells throughout the brain and spinal cord, making neurological diseases amenable to gene therapy approaches. Essential to the translation of this approach into the clinic is the identification of safe and effective routes for AAV delivery into the CSF of large animal models.

In this study, we directly compared the biodistribution and transduction efficiency of AAV9 across five different routes of CSF delivery at a controlled dose: unilateral Intracerebroventricular (ICV), bilateral ICV, intrathecal lumbar (IT-lumbar), and intracisterna magna (ICM) routes in juvenile neutralizing antibody (NAb) negative male cynomolgus macaques (Macaca fascicularis). Intra-CSF routes were additionally compared to intravenous (IV) injection at a similar dose. We also systematically quantified biodistribution and transduction efficiency of clinically-validated AAV serotypes, including AAV serotype 9 (AAV9), AAV serotype 5 (AAV5) and AAV serotype 1 (AAV1) via ICV administration.

We used AAV vectors expressing green fluorescent protein (eGFP) driven by a chicken beta actin promoter (CBA) via triple transfection of HEK293 cells. Vectors were titered via digital droplet PCR (ddPCR). Biodistribution was evaluated across CNS tissues and peripheral organs.

Thus, in this multi layered study, we demonstrate the efficacy of various routes of administration and AAV serotypes to target viral delivery to various brain structures. Our findings inform the selection of an intra-CSF route of administration and AAV capsid serotype selection for clinical translation of CNS-directed gene therapy.

### Example 1: Route of Administration Study in Cynomologus Monkeys

The objective of this study was to compare the biodistribution in the central nervous system (CNS) of cynomolgus macaque monkeys across five different Routes of administration: unilateral intracerebroventricular (ICV), bilateral ICV, intrathecal (IT) lumbar, intracisternal magna (ICM), or intravenous (IV) injection. Each animal was injected with AAV9 containing an expression cassette encoding eGFP-KASH under the control of a chicken beta actin (CBA) promoter (called AAV9-CBA-eGFP-KASH). The AAV9 particles were formulated in PBS+0.001% PF-68 and administered at either a high dose (1.0E+13 vg/animal) or a low dose (2.4E+12 vg/animal). A volume of 2 ml of formulated viral particles was administered to each animal regardless of route of administration. The study design is set forth below in Table 1.

TABLE 1
Study design for route of administration study.

| | | | | | Dose |
|---|---|---|---|---|---|
| Animal# | Group | Vector | ROA | Terminal Day | (VG/animal) |
| 4001 | 1A | AAV9-CBA-eGFP-KASH | Unilateral ICV | 28 | 1.0E+13 |
| 1005 | 1A | | Unilateral ICV | 28 | 2.4E+12 |
| 1002 | 1B | | Bilateral ICV | 29 | 1.0E+13 |
| 1003 | 1B | | Bilateral ICV | 29 | 2.4E+12 |
| 1006 | 1C | | IT | 27 | 1.0E+13 |
| 1001 | 1C | | IT | 27 | 2.4E+12 |
| 1008 | 1D | | ICM | 29 | 1.0E+13 |
| 1007 | 1D | | ICM | 28 | 2.4E+12 |
| 1009 | 1E | | IV | 27 | 2.5E+12 |

Experimentally naïve male cynomolgus monkeys (Macaca fascicularis) were used in this study. At the initiation of dosing, the animals were 10 to 11 months old and weighed 1.4±0.2 kg. Animals were assigned to study groups by a simple randomization procedure. Prior to initiation of the study, blood samples from the animals were tested for levels of neutralizing antibody (Nab) titer to AAV9, AAV5, and AAV1. Animals with low or negative results for antibodies were selected for the study.

### Intracerebroventricular Administration

The animals were anesthetized, prepared for surgery and mounted in a MRI compatible stereotaxic frame (Kopf). A baseline MRI was performed to establish target coordinates. An incision was made and a single hole was drilled through the skull over the target location. The needle was lowered into place and the AAV9-CBA-eGFP-KASH vector was infused into the lateral ventricle. Contrast media injections and fluoroscopy were used to verify needle placement into the ventricle. The AAV9-CBA-eGFP-KASH was infused at a rate of 0.1 mL/minute for 10 minutes for each the left and right bilateral ICV treatment and 0.1 mL/minute for 20 minutes for unilateral ICV treatment. The needle remained in place for between 1 to 2 minutes after the completion of the infusion. Following completion of dosing, the skin was closed in a standard manner and the animals were allowed to recover.

### Intrathecal (IT) Lumbar Injection

The animals were anesthetized with Isoflurane and placed in a lateral recumbency. The lumbar cistern was accessed via a percutaneous needle stick. The needle was inserted between L3/L4 as verified by contrast dye fluoroscopy. After placing the needle, positive CSF flow was confirmed. The syringe containing AAV9-CBA-eGFP-KASH was attached to the needle and the vector slowly infused by hand over 1 minute. After completion of the injection, the syringe was removed and CSF flow confirmed. Animals were placed in Trendelenburg position for 10 minutes following the completion of dosing.

### Intracisternal Magna (ICM) Injection

Animals were anesthetized with Isoflurane and placed in a lateral recumbency. The cisterna magna was accessed via a percutaneous needle stick. The needle was inserted between the base of the skull and C1. The syringe containing AAV9-CBA-eGFP-KASH was attached to the needle and the vector slowly infused by hand over 1 minute. After completion of the injection, the syringe was removed and CSF flow confirmed.

### Intravenous Injection

Animals were injected with AAV9-CBA-eGFP-KASH using a bolus injection into the tail vein.

Following dosing, animals were routinely monitored throughout the duration of the study and blood samples were withdrawn weekly. The following parameters and endpoints were evaluated: mortality, clinical observations, body weight, physical examinations, clinical pathology parameters (clinical chemistry), Neutralizing Antibody sample analysis, PBMC, CSF, biodistribution and gene expression analysis, gross necropsy findings, and histopathologic examinations.

The results of this study demonstrated that administration of the test article was not associated with any unexpected mortality, clinical findings, changes in body weights, or macroscopic observations. Upon evaluation of clinical chemistry endpoints, all animals administered AAV9-CBA-eGFP, regardless of route of administration, had increases in individual alanine aminotransferase (ALT), aspartate aminotransferase (AST), and/or glutamate dehydrogenase (GLDH) activities, which were considered AAV vector-related and indicative of hepatocellular effects.

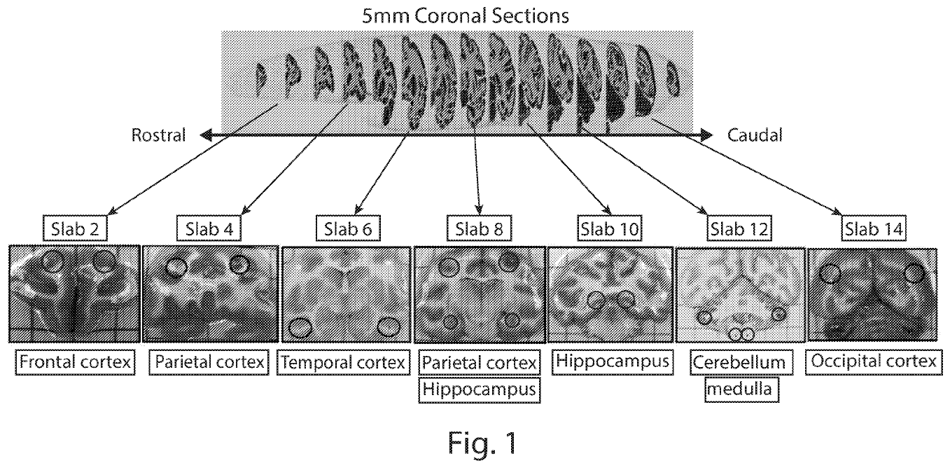

All animals survived to the scheduled necropsy. Following euthanasia and saline perfusion, the brain was removed and cut into 4 to 5 mm coronal sections (see FIG. 1), and the qPCR samples collected from even slabs, bilaterally, using an 8 mm biopsy punch. A new punch used for each site. Each biopsy punch was cut in half (one half for qPCR and the other half for RT-qPCR). Tissue samples collected from the brain included: 4 cortex regions ((frontal, parietal, temporal, and occipital) 2 sections when possible), hippocampus (2 sections when possible), medulla, and cerebellum.

For biodistribution studies using qPCR, tissue samples (100 to 200 mg per tissue sample with the exception of the spleen) were collected from the heart, liver, lungs, kidney (both), brain, spinal cord (SC), dorsal root ganglia (DRG), testes, and spleen (50 to 100 mg). Spinal cord and DRG's collected from cervical (C2), thoracic (T1 and T8), and lumbar (L4) regions. Samples were collected in individually prelabeled cryotubes, snap frozen in liquid nitrogen, and placed on dry ice. Samples were stored frozen at −60° C. to −90° C.

For gene expression studies using RT-PCR, tissue samples were collected from the heart, liver, lungs, kidney (both), spleen, lymph node, brain, spinal cord, DRG, and testes. Spinal cord and DRG's collected from (C3, C4, T2, T3, T9, T10, L2, and L5). Samples were individually placed in prelabeled cryotubes containing RNA-Later and refrigerated (2° C. to 8° C.) for 24 to 48 hours. Samples were removed from refrigeration and stored frozen at −60° C. to −90° C.

Histopathology Tissue Collection. Following qPCR and RT-qPCR sample collections, all remaining brain tissue, spinal cord, and DRG's, peripheral organs (lungs inflated with 4%) were fixed in 4% paraformaldehyde (PFA) for 24 to 48 hours at room temperature and then transferred to 70% ethanol.

### Vector Copy Number Assay

Vector copy number (VCN) was determined in various brain regions, spinal cord, dorsal root ganglion, heart, liver, kidney and spleen. For brain samples, tissue punches (see FIG. 1) from various brain regions, e.g., frontal cortex (2 punches, 1 from each hemisphere of slab 2), parietal cortex (4 punches, 1 from each hemisphere of slabs 4 and 8), temporal cortex (2 punches, 1 from each hemisphere of slab 6), hippocampus (4 punches, 1 from each hemisphere of slabs 8 and 10), cerebellum (2 punches, 1 from each hemisphere of slab 12), medulla (2 punches, 1 from each hemisphere of slab 12), and occipital cortex (2 punches, 1 from each hemisphere of slab 14) were used. All tissue samples were processed as set forth below.

Tissue DNA was isolated with DNeasy Blood & Tissues kits (Qiagen). DNA quantity was determined and normalized using UV spectrophotometer. 100 ng of tissue DNA was added to a 50 μl reaction along with TaqPath ProAmp Multiplex Master Mix (Thermo Fisher Scientific) and TaqMan primers and probes directed against regions of eGFP. The plasmid standard curves were prepared by restriction enzyme linearization and purification with a DNA Clean & Concentrator kit (Zymo Research). The linearized DNA was quantified by UV spectrophotometry and 10-fold serially diluted from 10⁶ to 50 copies per 10 μl. Diluted standard curves were added into 50 μl reaction as for the tissue samples. TaqMan qPCR was performed using the Lightcycler 96 system (Roche, Life Science) to determine vector copy number in tissues for biodistribution studies, using a two-step cycling protocol (initial denature/enzyme activation: 95° C. for 10 minutes, 40 cycles: 95° C. for 15 seconds, 60° C. for 60 seconds). Monkey genomic albumin (Alb) sequence served as an internal control for genomic DNA content and was amplified in a separate reaction. Samples were considered eligible if the Alb Ct value was less than 26.

| eGFP primers probe sequences: | |
|---|---|
| FW: | AACCGCATCGAGCTGAAGG; |
| RV: | GCCATGATATAGACGTTGTGGC; |
| Probe: | AGGAGGACGGCAACATCCTGGGGCA |
| Cynomolgus monkey albumin sequences: | |
| FW: | GCTGTTATCTCTTGTGGGCTGT |
| RV: | AAACTCATGGGAGCTGCCGGTT |
| Probe: | CCACACAAATCTCTCCCTGGCATTG |

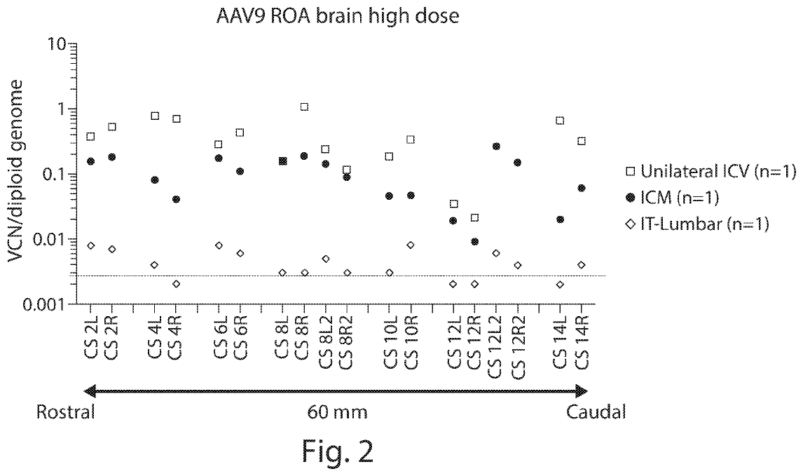

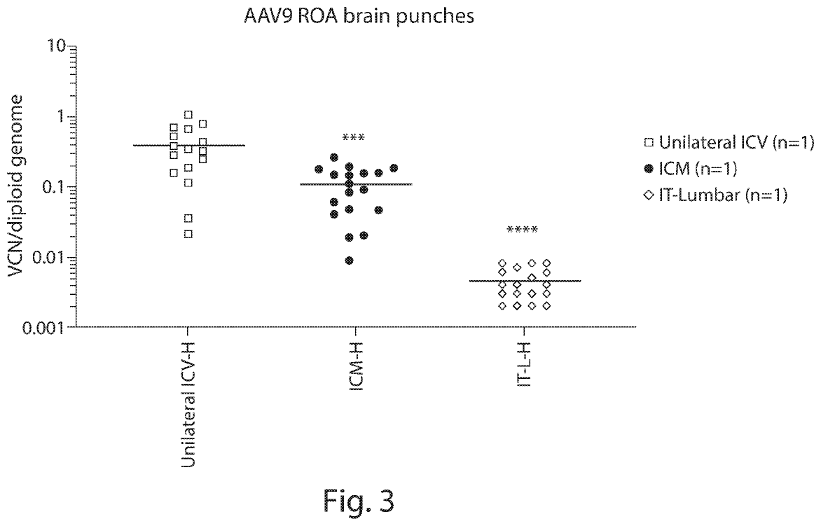

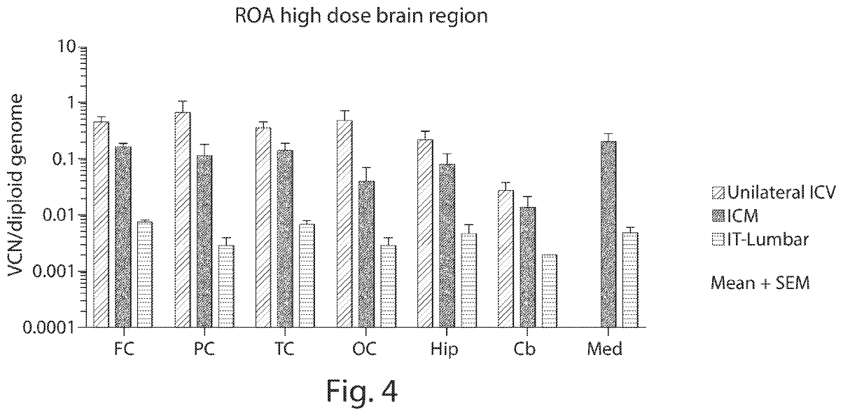

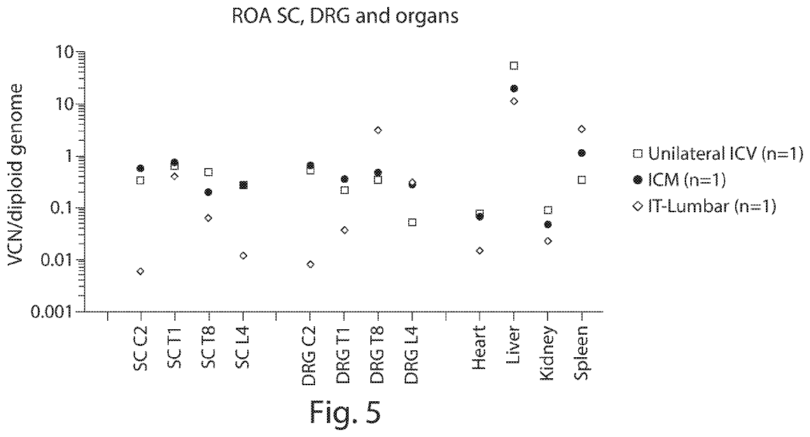

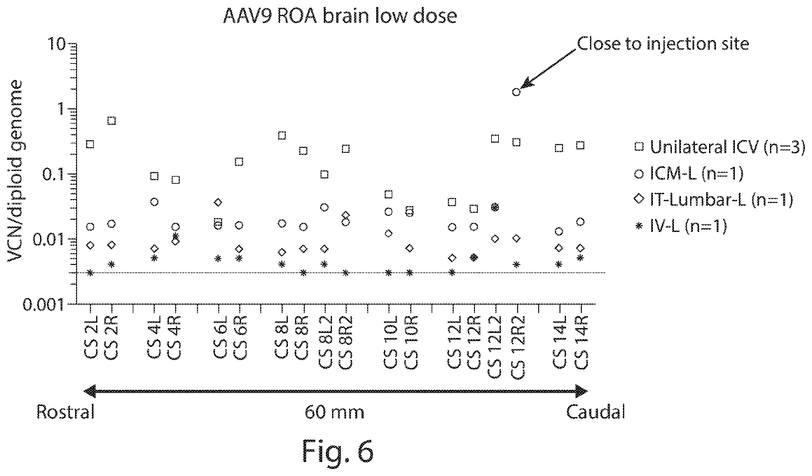

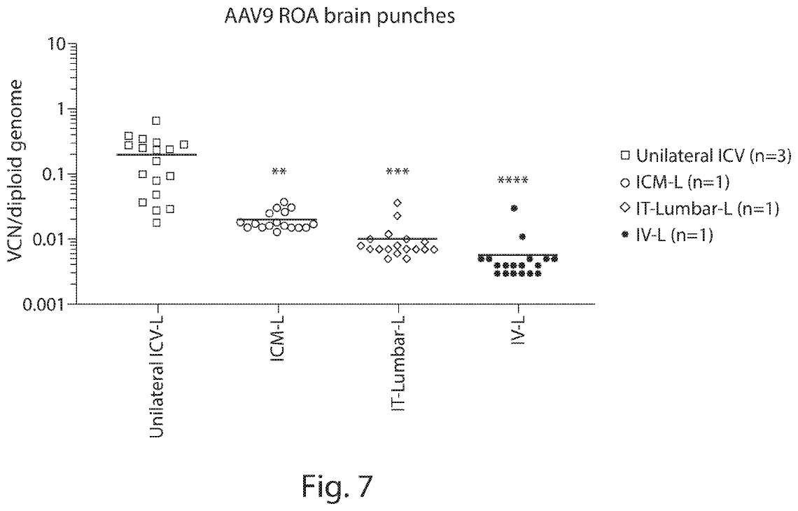

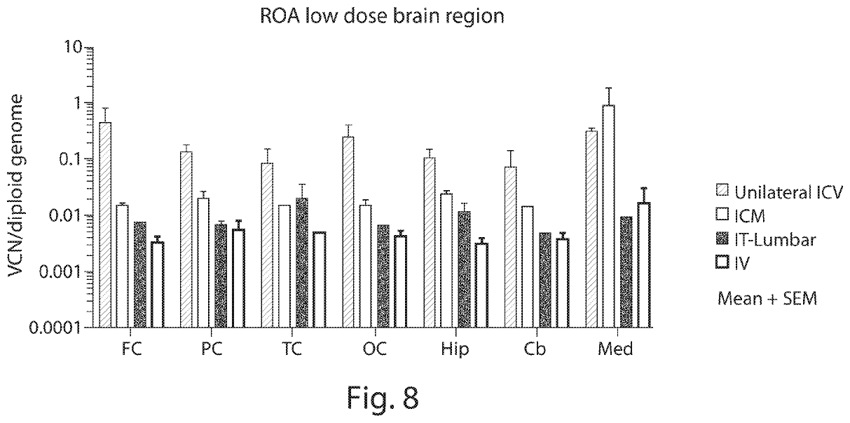

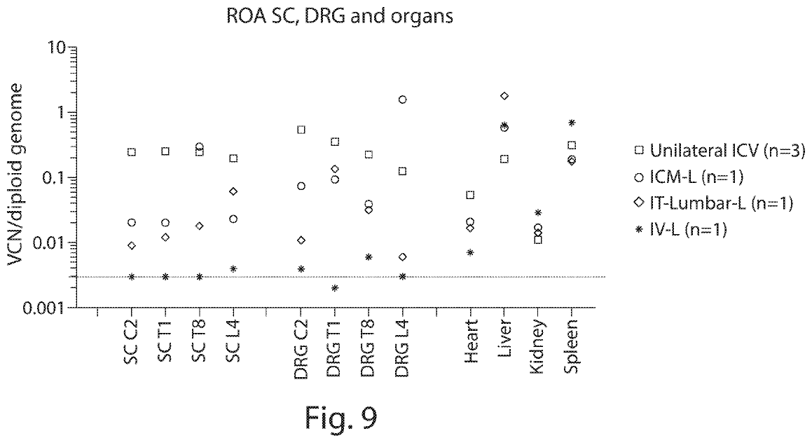

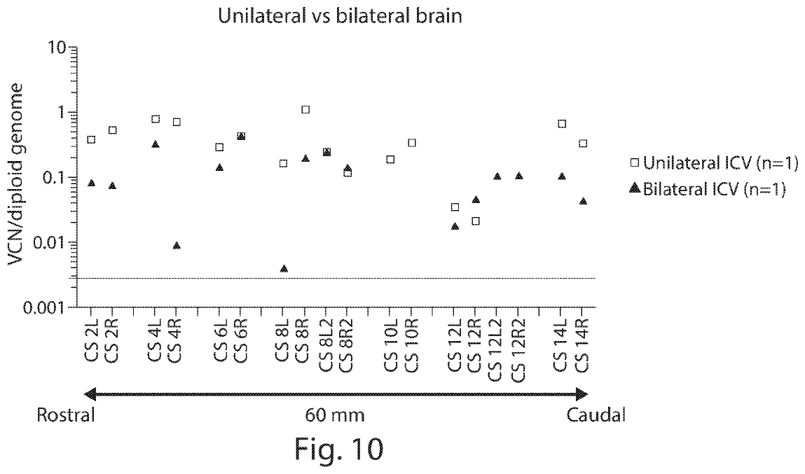

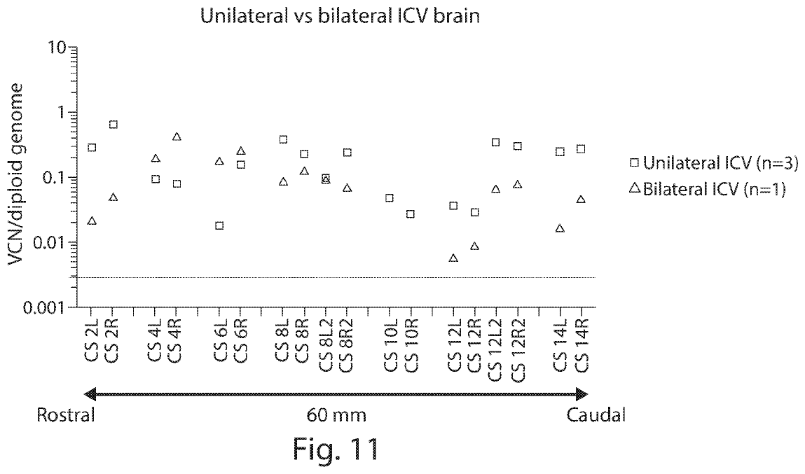

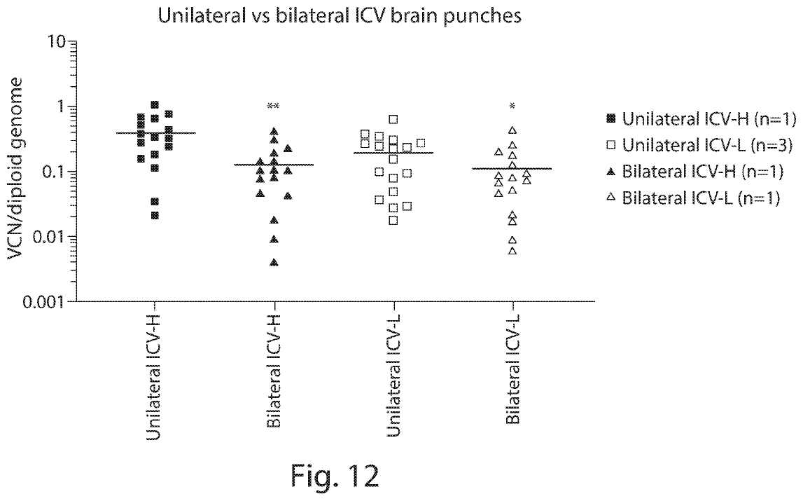

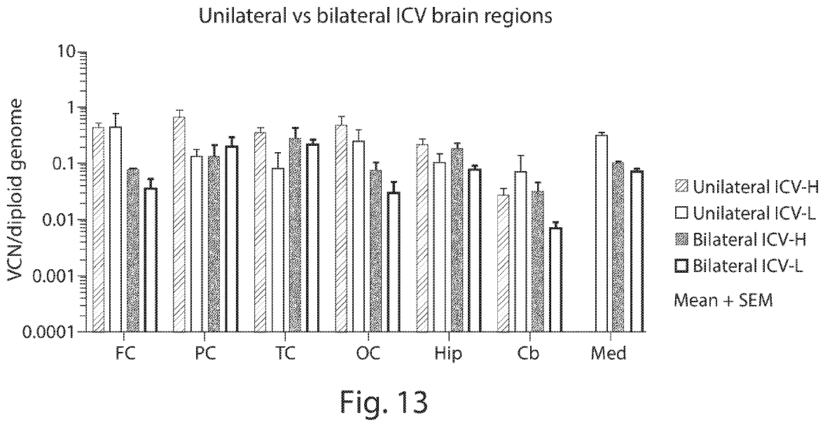

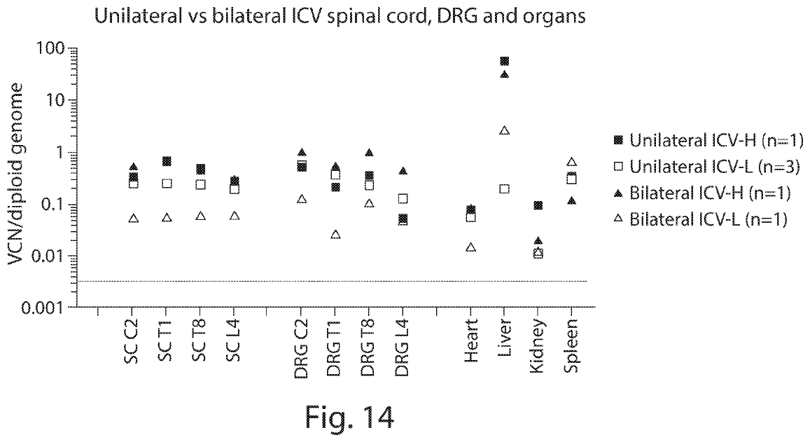

The results of the vector copy number assay show that ICV administration is more efficient at delivering AAV to the brain than ICM administration and ICV is significantly more efficient at delivering AAV to the brain than IT-lumbar or IV administration (see FIGS. 2-9). In addition, the results show that unilateral ICV administration is comparable or more efficient at delivery of AAV to the brain than bilateral ICV administration (see FIGS. 10-14).

### Determination of Anti-AAV Neutralizing Antibody (NAb) Titer in Non-Human Primate Sera

The titer of neutralizing antibody following before and after treatment with viral vectors was determined. The 293AAV Cell Line was purchased from Cell Biolabs, Inc. (San Diego, Calif.) and cultured in DMEM supplemented with 10% Heat-inactivated FBS. Nano-Glo® Luciferase Assay System and GloMax®-Multi+ Microplate Multimode Reader (Promega (Madison, Wis.)) were used. NHP sera were obtained from blood draws obtained pre-dose and at days 1, 14 and 28 after dosing. The serum samples were heat-inactivated at 56° C. for 30 minutes prior to use.

On day-1 of the assay, 293AAV cells were plated in a 96-well flat-bottom culture plate at 1×10⁴/100 ul (AAV1 and AAV5) or 1.5×10⁴/100 ul (AAV9), and incubated overnight at 37° C., 5% CO₂. On day-2, serial dilutions of NHP serum samples were made before mixing the samples with AAV-CMV_NLuc vectors and incubated at 37° C. for 1 hour. An 100% vector transduction control and 0% transduction (signal background) control were also generated for each plate. Finally, co-incubated mixtures were transferred to the 96-well flat-bottom culture plate to reach MOI (multiplicity of infection) of 1000, 2000 and 10000 for AAV1, AAV5 and AAV9 respectively. After a 48-hour incubation at 37° C., Nano-Glo® Luciferase Assay Reagent was prepared per manufacture instruction and added to the plate, and luminescence was measured in Greiner Bio-One White Polystyrene LUMITRAC 200 Microplate (Greiner Bio-One)).

The results of the assay are shown in Table 2 below. Anti-AAV neutralizing antibody titer is defined as the reciprocal of the highest serum dilution at which AAV transduction was reduced by >50% compared to negative control.

$$\% Inhibition = 100 - ( \frac{RLU test sample - RLU no virus}{RLU max - RLU no Virus} \times 100 )$$

Post AAV9 vector administration all animals had measurable anti AAV9 capsid neutralizing antibodies that were sustained until the end of study (see Table 2).

TABLE 2
Neutralizing antibody titers for animals treated with AAV9 vectors.

| | | | | Dose | Neutralizing Antibody Titer | | | |
|---|---|---|---|---|---|---|---|---|
| Animal# | Group | Vector | ROA | (VG/animal) | Pre-dose | Day 1 | Day 14 | Day 28 |
| 4001 | 1A | AAV9- | Unilateral | 1.0E+13 | <5 | <5 | 80 | 80 |
| | | CBA- | ICV | | | | | |
| 1005 | 1A | eGFP- | Unilateral | 2.4E+12 | <5 | <5 | 320 | 1280 |
| | | KASH | ICV | | | | | |
| 1002 | 1B | | Bilateral ICV | 1.0E+13 | <5 | <5 | 80 | 80 |
| 1003 | 1B | | Bilateral ICV | 2.4E+12 | <5 | <5 | 320 | 80 |
| 1006 | 1C | | IT | 1.0E+13 | <5 | <5 | 80 | 80 |
| 1001 | 1C | | IT | 2.4E+12 | <5 | <5 | 80 | 80 |
| 1008 | 1D | | ICM | 1.0E+13 | <5 | <5 | 20 | 5 |
| 1007 | 1D | | ICM | 2.4E+12 | <5 | <5 | 320 | 1280 |
| 1009 | 1E | | IV | 2.5E+12 | 40 | 5 | 320 | 320 |
| 11004 | 4 | AAV9- | Unilateral | 2.7E+12 | 40 | 80 | 20 | 80 |
| | | SEQ ID | ICV | | | | | |
| 4002 | 4 | 76 - | Unilateral | 2.7E+12 | <5 | 5 | 80 | 80 |
| | | eGFP- | ICV | | | | | |
| | | WPRE | | | | | | |

### Immunohistochemistry Assay for Route of Administration Study

The level of green fluorescent protein (GFP) expression in various tissues was determined following AAV administration. Following saline perfusion, tissue was fixed in 4% paraformaldehyde for 48 hours, transferred to 70% ethanol, paraffin embedded and sectioned at 5 μm. After removing the paraffin with xylene and alcohol, heat retrieval was performed in citrate buffer (pH 6) for 20 min at 95° C. Primary antibody staining with chicken anti-GFP (Ayes Labs GFP10201) was performed overnight at 1:5000 then detected with goat anti-chicken-HRP (Thermo A16054) at 1:1000 for 1 hr. TSA-FITC (PerkinElmer) was used at 1:100 for 10 min followed by DAPI staining. Slides were imaged with a PE Vectra3 using a 10× objective and images of DAPI and FITC staining was taken at 4 and 40 ms respectively.

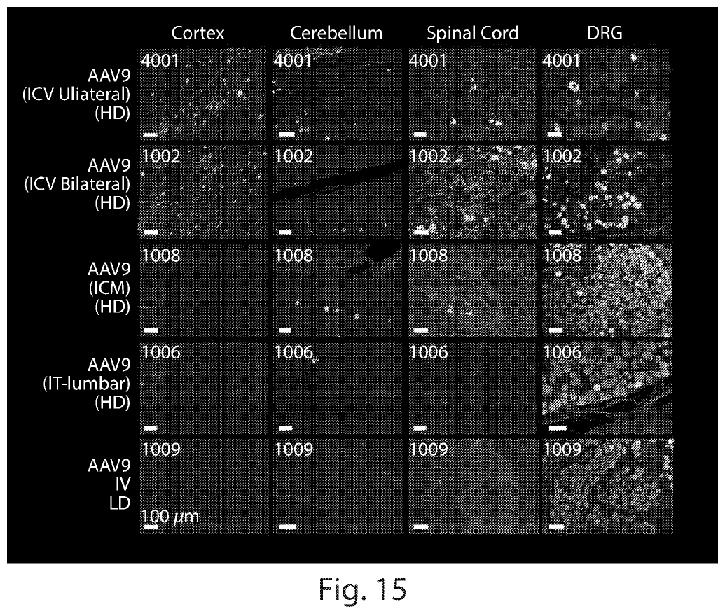

As shown in FIG. 15, animals dosed with AAV9 vectors administered by different routes of administration show different extents of GFP expression in the brain regions, spinal cord and dorsal root ganglia.

### Example 2: AAV Serotype Study in Cynomologus Monkeys

The objective of this study was to compare the biodistribution in the central nervous system (CNS) of cynomolgus macaque monkeys using 3 different AAV serotypes: AAV1, AAV5 and AAV9. The animals were injected with an AAV vector (either AAV1, AAV5 or AAV9) containing an expression cassette encoding eGFP-KASH under the control of a chicken beta actin (CBA) promoter (called AAVX-CBA-eGFP-KASH) or an AAV9 vector containing an expression cassette encoding eGFP under the control of a promoter having SEQ ID NO: 76 and containing a woodchuck hepatitis virus posttranscriptional regulatory element (WPRE) (called AAV9-SEQ ID 76-eGFP-WPRE). The AAV particles were formulated in PBS+0.001% PF-68 and administered at the dose listed in the table below. A volume of 2 ml of formulated viral particles was administered to each animal. The study design is set forth below in Table 3.

TABLE 3
Study design for AAV serotype study.

| | | | | Terminal | Dose |
|---|---|---|---|---|---|
| Animal# | Group | Vector | ROA | Day | (VG/animal) |
| 2001 | 2 | AAV5-CBA- | Unilateral ICV | 30 | 2.8E+12 |
| 2002 | 2 | eGFP-KASH | Unilateral ICV | 30 | 2.8E+12 |
| 3001 | 3 | AAV1-CBA- | Unilateral ICV | 28 | 2.0E+12 |
| 3002 | 3 | eGFP-KASH | Unilateral ICV | 14 | 2.0E+12 |
| 11004 | 4 | AAV9-SEQ | Unilateral ICV | 29 | 2.7E+12 |
| 4002 | 4 | ID 76-eGFP- | Unilateral ICV | 29 | 2.7E+12 |
| | | WPRE | | | |

The animals were dosed as set forth in Example 1 for unilateral ICV injection. Animals were routinely monitored and blood samples withdrawn weekly as set forth in Example 1. All animals survived to the scheduled necropsy with the exception of animal 3002. On Day 14, animal 3002 was noted to be ataxic with decreased and abnormal activity. The animal continued to decline and was euthanized.

All animals administered AAV9-CBA-eGFP, regardless of route of administration or lot, and a few individuals administered AAV5-CBA-eGFP or AAV1-CBA-eGFP, had increases in individual alanine aminotransferase (ALT), aspartate aminotransferase (AST), and/or glutamate dehydrogenase (GLDH) activities, which were considered AAV vector-related and indicative of hepatocellular effects. Similar effects were not observed following AAV9-SEQ ID 76-eGFP-WPRE administration.

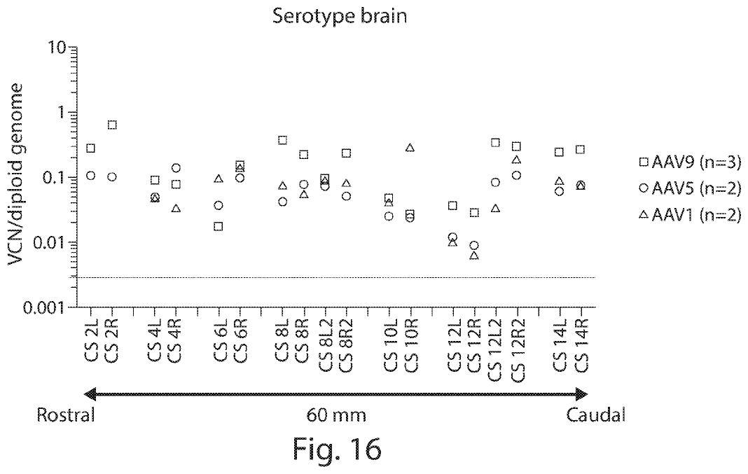

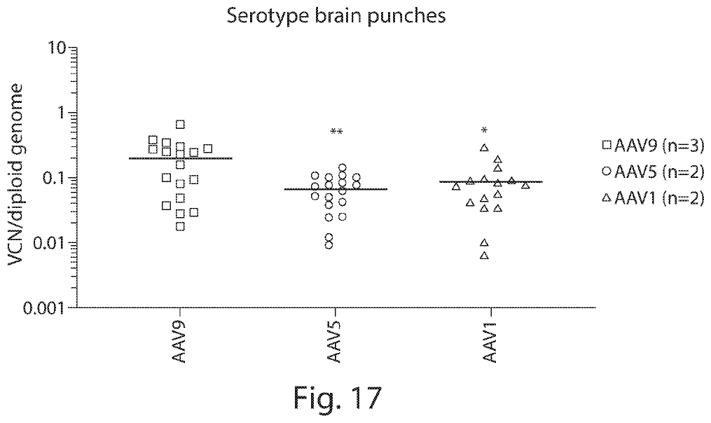

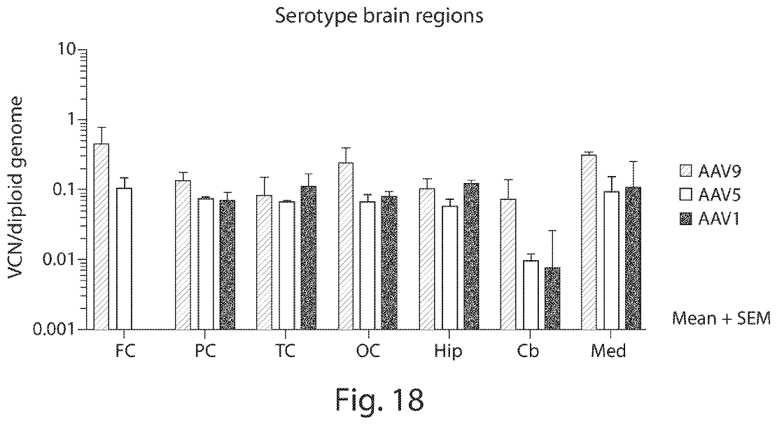

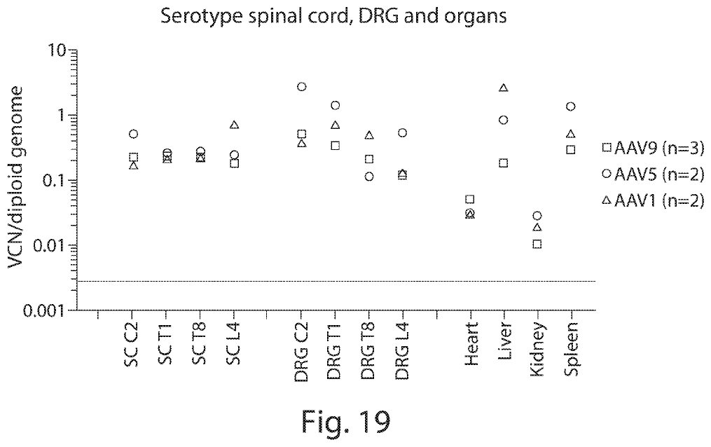

Following euthanasia, tissues were processed for qPCR, RT-qPCR and histopathology as set forth in Example 1. Vector copy number was determined as described in Example 1. The results show that AAV1, AAV5 and AAV9 showed comparable vector transduction in the brain, although the AAV9 levels were slightly higher (see FIGS. 16-19).

Neutralizing antibody titers were also determined for the serotype study as set forth above in Example 1. The results for AAV9 vectors are shown above in Table 2 and the results for AAV5 and AAV1 vectors are shown below in Table 4.

TABLE 4
Neutralizing antibody titers for animals treated with AAV5 and AAV1 vectors.

| | | | | Dose | Neutralizing Antibody Titer | | | |
|---|---|---|---|---|---|---|---|---|
| Animal# | Group | Vector | ROA | (VG/animal) | Pre-dose | Day 1 | Day 14 | Day 28 |
| 2001 | 2 | AAV5- | Unilateral | 2.8E+12 | <5 | <5 | 5120 | 5120 |
| | | CBA- | ICV | | | | | |
| 2002 | 2 | eGFP- | Unilateral | 2.8E+12 | <5 | <5 | 5120 | 5120 |
| | | KASH | ICV | | | | | |
| 3001 | 3 | AAV1- | Unilateral | 2.0E+12 | <5 | 5 | 1280 | 320 |
| | | CBA- | ICV | | | | | |
| 3002 | 3 | eGFP- | Unilateral | 2.0E+12 | <5 | 20 | 80 | N/A |
| | | KASH | ICV | | | | | |

Post AAV1, AAV5 and AAV9 vector administration all animals had measurable anti AAV capsid neutralizing antibodies that were sustained until the end of study (see Tables 2 and 4).

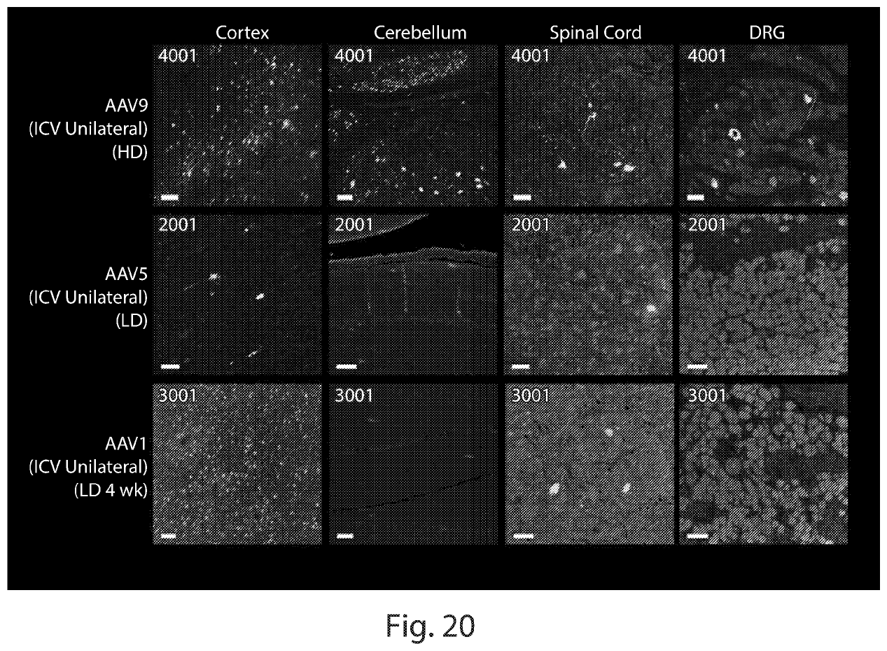

IHC analysis of GFP expression levels was also determined for animals treated with AAV1, AAV5 and AAV9 as set forth above in Example 1. As shown in FIG. 20, a varied extent of GFP expression was observed across all three serotypes in the brain and spinal cord tissues.

### Example 3: eTF^{SCN1A} Biodistribution

The objective of this study was to compare the biodistribution of eTF^{SCN1A} in the central nervous system (CNS) of juvenile cynomolgus macaque monkeys when administered at a dose of 4.8E+13 vg/animal or 8E+13 vg/animal via unilateral intracerebroventricular (ICV) injection. Each animal was injected with AAV9 containing an expression cassette encoding eTF^{SCN1A} under the control of a GABA selective regulatory element (RE^{GABA}-eTF^{SCN1A}). The AAV9 particles were formulated in PBS+0.001% pluronic acid and administered at a dose of 4.8E+13 vg/animal or 8E+13 vg/animal. A volume of 2 ml of formulated viral particles was administered to each animal. The study design is set forth in Table 8.

Twenty-four month old cynomolgus macaque monkeys were grouped as indicated in Table 8. Prior to initiation of the study, blood samples from the animals were tested for levels of neutralizing antibody titer to AAV9 using the NAb titer assay described above. Animals with low or negative results for antibodies were selected for the study. Samples were administered via ICV injection using standard surgical procedures. Thawed dosing material was briefly stored on wet ice and warmed to room temperature just prior to dosing. The animals were anesthetized, prepared for surgery, and mounted in a MRI compatible stereotaxic frame (Kopf). A baseline MRI was performed to establish target coordinates. An incision was made and a single hole was drilled through the skull over the target location. A 3 mL BD syringe attached to a 36″ micro-bore extension set was prepared with sample and placed in an infusion pump. The extension line was primed. The dura was opened, and the dosing needle was advanced to a depth of 13.0 to 18.1 mm from the pia. Contrast media injection and fluoroscopy was used to confirm placement of the spinal needle into the right lateral ventricle. The 3.0″ 22 g Quinke BD spinal huber point needle was filled with contrast to determine placement prior to attaching the primed extension line and syringe. Pump settings were 0.1 mL/minute for 19 to 20 minutes. Buffer was pushed by hand post dose to clear the extension line. The needle remained in place for 1 to 2 minutes post completion of infusion and then the needle was withdrawn. The vehicle and test article were administered once on day 1 and the subjects were maintained for a 27- or 29-day recovery period.

TABLE 8
Biodistribution Study design

| Group | Gender | ID | Dose (VG/animal) |
|---|---|---|---|
| Group 1 | M | 21001 | |
| (Buffer Control) | F | 11501 | |
| Group 2 | M | 2001 | 4.8E+13 |
| (RE^{GABA}-eTF^{SCN1A}) | F | 2501 | 4.8E+13 |
| | M | 3001 | 4.8E+13 |
| | M | 3002 | 8E+13 |

Following dosing, animals were routinely monitored throughout the duration of the study and blood samples were periodically withdrawn. eTF^{SCN1A} administration was not associated with any unexpected mortality, clinical findings, or macroscopic observations. AAV9-RE^{GABA}-eTF^{SCN1A} treated animals survived until scheduled necropsy at day 28±2 days. No clinical or behavioral signs, increases in body temperature, or body weight reduction were observed during daily or weekly physical examinations. Transient elevation in liver transaminases (ALT and AST) in AAV9-RE^{GABA}-eTF^{SCN1A} treated animals were observed, but were fully resolved by the end of study without immunomodulation, and no concomitant increase in serum bilirubin or alkaline phosphatases was noted. No other measured clinical chemistry endpoint was remarkable. No microscopic observations were reported in the liver histopathology studies. CSF leukocytes were elevated in terminal collection relative to pre-treatment values but comparable between control and AAV9-RE^{GABA}-eTF^{SCN1A} treated animals. No AAV9-RE^{GABA}-eTF^{SCN1A} associated pleocytosis was observed. Macro-observations and detailed micro-histopathology examination of non-neuronal tissues across all animals were unremarkable. Tissues included major peripheral organs (i.e. heart, lungs, spleen, liver and gonads). Macro-observations and detailed micro-histopathology of neuronal tissues did not show any notable findings. Tissues included brain, spinal cord, and associated dorsal root ganglia (from cervical, thoracic and lumbar region). Studies were conducted by three independent pathologists including one at a specialized neuropathology site.

ICV administration of AAV9 did not prevent post-dose immune response in the serum, as anti-AAV9 capsid neutralizing antibodies were observed four weeks post-dose. However, neutralizing anti-AAV9 antibody levels in the CSF remained unchanged and comparable to pre-dose levels (Table 9).

TABLE 9
AAV9 serum NAb titer

| | AAV9 Serum NAb Titer | | | AAV9 CSF NAb Titer | |
|---|---|---|---|---|---|
| | | | 4-Weeks | | 4-Weeks |
| Subject | | | Post | | Post |
| Number | Pre-Screen | At Injection | Injection | At Injection | Injection |
| 21001 | 1:5 | <1:5 | <1:5 | <1:5 | <1:5 |
| 11501 | <1:5 | <1:5 | <1:5 | <1:5 | <1:5 |
| 2001 | <1:5 | <1:5 | 1:405 | <1:5 | 1:5 |
| 2501 | <1:5 | <1:5 | 1:135 | <1:5 | 1:5 |
| 3001 | <1:5 | <1:5 | 1:1215 | <1:5 | <1:5 |
| 3002 | <1:5 | <1:5 | 1:135 | <1:5 | <1:5 |

Samples were collected 27-29 days post-dose from major organs (heart ventricles, liver lobes, lung cardiac lobes, kidneys, spleen, pancreas, and cervical lymph nodes) during schedule necroscopy. Punches were collected via eight millimeter punch and further processed as discussed below.

### Example 4: Biodistribution of eTF^{SCN1A} in the Brain

ddPCR was used to measure eTF^{SCN1A} biodistribution in the brain. Samples from various regions of cynomolgus macaque brain tissue (FC: Frontal cortex; PC: parietal cortex; TC: temporal cortex; Hip: hippocampus; Med: medulla; OC: occipital cortex) were measured for vector copy number to assess biodistribution of eTF^{SCN1A} under the control of a GABA selective regulatoryelement (RE^{GABA}-eTF^{SCN1A}) when administered in AAV9 by unilateral ICV. Tissue DNA was isolated with DNeasy Blood & Tissues kits (Qiagen). DNA quantity was determined and normalized using UV spectrophotometer. 20 nanograms of tissue DNA was added to a 20 microliter reaction along with ddPCR Super Mix for Probes (no dUTP) (Bio-Rad) and TaqMan primers and probes directed against regions of the eTF^{SCN1A} sequence. Droplets were generated and templates were amplified using automated droplet generator and thermo cycler (Bio-Rad). After the PCR step, the plate was loaded and read by QX2000 Droplet Reader to determine vector copy number in tissues. Monkey Albumin (MfAlb) gene served as an internal control for normalizing genomic DNA content and was amplified in the same reaction. Primers and probes for eTF^{SCN1A} and MfAlb are set forth in Table 10.

TABLE 10
Primers and probes for eTF^{SCN1A} and MfAlb

| Primers/ | | |
|---|---|---|
| probe | | |
| Name | | Sequence (5′-3′) |
| eTF^{SCN1A} | eTF^{SCN1A} Forward | GAATGTGGGAAATCATTCAGTCGC |
| | primer | (SEQ ID NO: 77) |
| | eTF^{SCN1A} Reverse | GCAAGTTATCCTCTCGTGAGAAGG |
| | primer | (SEQ ID NO: 78) |
| | eTF^{SCN1A} probe | GCGACAACCTGGTGAGACATCAAC |
| | | GCACC (SEQ ID NO: 79) |
| MfAlbumin | MfAlb Forward | GCTGTTATCTCTTGTGGGCTGT |
| | primer | (SEQ ID NO: 80) |
| | MfAlb Reverse | AAACTCATGGGAGCTGCCGGTT |
| | primer | (SEQ ID NO: 81) |
| | MfAlb probe | CCACACAAATCTCTCCCTGGCATTG |
| | | (SEQ ID NO: 82) |

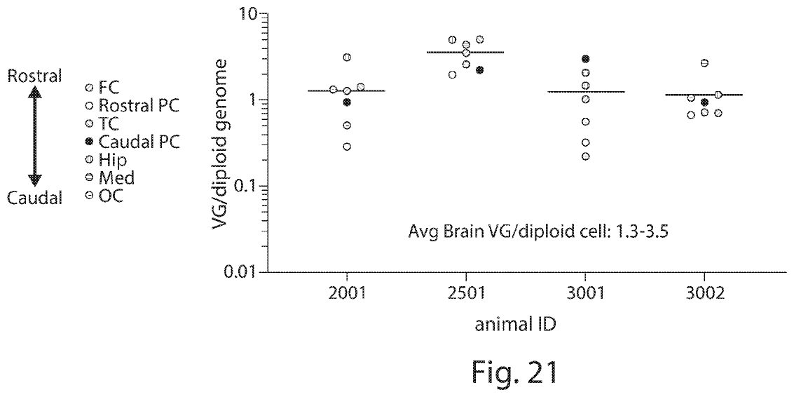

eTF^{SCN1A} was broadly distributed throughout the brain when dosed at 4.8E+13 viral genomes per animal with an average of 1.3-3.5 VG/diploid genome (FIG. 21). In addition, when comparing gene transfer throughout the brain of RE^{GABA}-eTF^{SCN1A} dosed at 4.8E+13 viral genomes per animal to gene transfer throughout the brain of eGFP dosed via ICV at various doses, an increase in VG/diploid genome was observed with increasing doses. This indicated that gene transfer in the brain occurred in a dose-dependent manner when administered in AAV9 via ICV.

### Example 5: eTF^{SCN1A} Transcription in the Brain

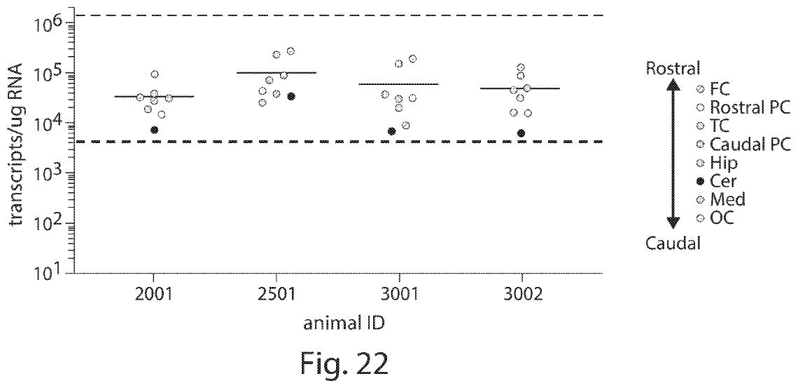

Transcription of eTF^{SCN1A} under the control of a GABA selective regulatory element, RE^{GABA} (RE^{GABA}-eTF^{SCN1A}), was assessed by measuring eTF^{SCN1A} mRNA using a ddPCR-based gene expression assay. Tissue RNA was isolated with RNeasy Plus Mini kits (Qiagen) or RNeasy Lipid Tissue Mini kits (Qiagen) for brain tissues. RNA quantity was determined and normalized using UV spectrophotometer and RNA quality (RIN) was checked using Bioanalyzer RNA Chip. One microgram of tissue RNA was used for DNase treatment and cDNA synthesis with SuperScript VILO cDNA synthesis kit with ezDNase™ Enzyme kits (Thermo Fisher). 50 micrograms of RNA was converted to cDNA. cDNA was added to a 20 microliter reaction along with ddPCR Super Mix for Probes (no dUTP) (Bio-Rad) and TaqMan primers and probes directed against regions of eTF^{SCN1A} sequence (Table 11). Droplets were generated and templates were amplified using automated droplet generator and thermo cycler (Bio-Rad). After PCR amplification, the plate was loaded and read by QX2000 Droplet Reader to provide gene expression levels in tissues. The monkey gene ARFGAP2 (MfARFGAP2) (Thermo Fisher Scientific) served as an endogenous control for normalizing gene expression levels and was amplified in the same reaction. Average transcripts for ARFGAP2 were 1.85E+6/ug RNA (FIG. 22, upper boundary). Limit of detection indicated by lower boundary.

eTF^{SCN1A} mRNA was observed throughout the brain in all animals, indicating that the GABA-selective promoter, RE^{GABA}, was transcriptionally active in the brain tissue for all AAV9-RE^{GABA}-eTF^{SCN1A} treated macaques (FIG. 22). FC: Frontal cortex; PC: parietal cortex; TC: temporal cortex; Hip: hippocampus; Med: medulla; OC: occipital cortex.

TABLE 11
TaqMan primers and probes directed against regions
of eTF^{SCN1A} sequence

| Primers/ | | |
|---|---|---|
| probe | | |
| Name | Description | Sequence (5′-3′) |
| eTF^{SCN1A} | eTF^{SCN1A} Forward | GAATGTGGGAAATCATTCAGTCGC |
| | primer | (SEQ ID NO: 83) |
| | eTF^{SCN1A} Reverse | GCAAGTTATCCTCTCGTGAGAAGG |
| | primer | (SEQ ID NO: 84) |
| | eTF^{SCN1A} probe | GCGACAACCTGGTGAGACATCAAC |
| | | GCACC (SEQ ID NO: 85) |
| MfARFGAP2 | Forward, Reverse | Thermo Fisher (Cat#: |
| | Primers, Probe | 4448491) |

### Example 6: eTF^{SCN1A} Biodistribution and Transcription in Peripheral Tissues

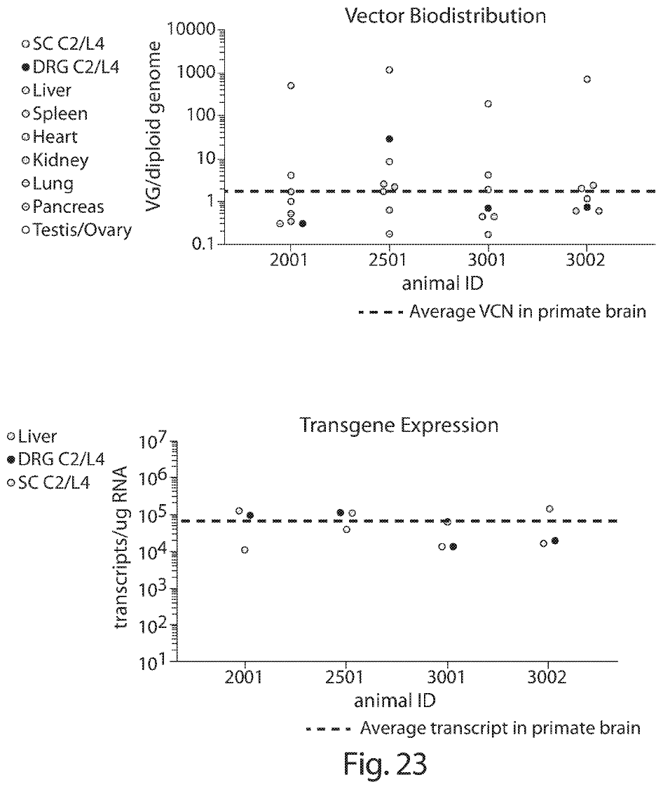

Vector copy number was further measured in various organs to evaluate transduction of RE^{GABA}-eTF^{SCN1A} in tissues throughout the body when administered in AAV9 by unilateral ICV. Transcript levels of eTF^{SCN1A} were also measured by ddPCR to assess transcriptional activity eTF^{SCN1A} under the control of the GABA-selective regulatory element RE^{GABA} in tissues throughout the body when administered in AAV9 by unilateral ICV. Both methods were performed as generally described above. RE^{GABA}-eTF^{SCN1A} transduction and transcription of eTF^{SCN1A} in the spinal cord (SC) and dorsal root ganglion (DRG) were comparable to levels observed in the brain. With the exception of the liver, RE^{GABA}-eTF^{SCN1A} transduction was lower in peripheral tissues outside of the brain (FIG. 23). Transduction of RE^{GABA}-eTF^{SCN1A} in the liver was higher than in the brain. Transcription of eTF^{SCN1A} was undetected in peripheral tissues, including the heart, lungs and gonads. However, eTF^{SCN1A} transcript levels in the liver were comparable to the levels of eTF^{SCN1A} measured in the brain. Furthermore, eTF^{SCN1A} transcription in the liver is extremely low when normalized to the number of vector copies present (approximately 1000-fold lower compared to transcription of eTF^{SCN1A} in the brain). Overall, this demonstrated that transcription of eTF^{SCN1A} under the control of the GABA-selective regulatory element RE^{GABA} is restricted to the CNS.

### I. Sequences

TABLE 5
List of exemplary regulateiy element nucleic acid sequences

| SEQ ID | | |
|---|---|---|
| NO: | Nucleic Acid Sequence | Length |
| 1 | GTAAGGTAAGAATTGAATTTCTCAGTTGAAGGATGCTTACACTC | 56 bp |
| | TTGTCCATCTAG | |
| 2 | GTGTGTATGCTCAGGGGCTGGGAAAGGAGGGGAGGGAGCTCCG | 49 bp |
| | GCTCAG | |
| 3 | GTGATGCGGTTTTGGCAGTACATCAATGGGCGTGGATAGCGGTT | 266 bp |
| | TGACTCACGGGGATTTCCAAGTCTCCACCCCATTGACGTCAATG | |
| | GGAGTTTGTTTTGGCACCAAAATCAACGGGACTTTCCAAAATGT | |
| | CGTAACAACTCCGCCCCATTGACGCAAATGGGCGGTAGGCGTG | |
| | TACGGTGGGAGGTCTATATAAGCAGAGCTGGTACCGTAAGGTA | |
| | AGAATTGAATTTCTCAGTTGAAGGATGCTTACACTCTTGTCCAT | |
| | CTAG | |
| 4 | GTGATGCGGTTTTGGCAGTACATCAATGGGCGTGGATAGCGGTT | 259 bp |
| | TGACTCACGGGGATTTCCAAGTCTCCACCCCATTGACGTCAATG | |
| | GGAGTTTGTTTTGGCACCAAAATCAACGGGACTTTCCAAAATGT | |
| | CGTAACAACTCCGCCCCATTGACGCAAATGGGCGGTAGGCGTG | |
| | TACGGTGGGAGGTCTATATAAGCAGAGCTGGTACCGTGTGTATG | |
| | CTCAGGGGCTGGGAAAGGAGGGGAGGGAGCTCCGGCTCAG | |
| 5 | GTGATGACGTGTCCCATAAGGCCCCTCGGTCTAAGGCTTCCCTA | 117 bp |
| | TTTCCTGGTTCGCCGGCGGCCATTTTGGGTGGAAGCGATAGCTG | |
| | AGTGGCGGCGGCTGCTGATTGTGTTCTAG | |
| 6 | GTGATGACGTGTCCCATACTTCCGGGTCAGGTGGGCCGGCTGTC | 117 bp |
| | TTGACCTTCTTTGCGGCTCGGCCATTTTGTCCCAGTCAGTCCGGA | |
| | GGCTGCGGCTGCAGAAGTACCGCCTGCG | |
| 7 | GTGATGACGTGTCCCATATTTTCATCTCGCGAGACTTGTGAGCG | 117 bp |
| | GCCATCTTGGTCCTGCCCTGACAGATTCTCCTATCGGGGTCACA | |
| | GGGACGCTAAGATTGCTACCTGGACTTTC | |
| 8 | GTGATGACGTGTCCCATGGCCTCATTGGATGAGAGGTCCCACCT | 117 bp |
| | CACGGCCCGAGGCGGGGCTTCTTTGCGCTTAAAAGCCGAGCCG | |
| | GGCCAATGTTCAAATGCGCAGCTCTTAGTC | |
| 9 | GTGATGACGTGTCCCATCCCCCCTCCACCCCCTAGCCCGCGGAG | 117 bp |
| | CACGCTGGGATTTGGCGCCCCCCTCCTCGGTGCAACCTATATAA | |
| | GGCTCACAGTCTGCGCTCCTGGTACACGC | |
| 10 | CCCCCCTCCACCCCCTAGCCCGCGGAGCACGCTGGGATTTGGCG | 100 bp |
| | CCCCCCTCCTCGGTGCAACCTATATAAGGCTCACAGTCTGCGCT | |
| | CCTGGTACACGC | |
| 11 | GGCCTCATTGGATGAGAGGTCCCACCTCACGGCCCGAGGCGGG | 100 bp |
| | GCTTCTTTGCGCTTAAAAGCCGAGCCGGGCCAATGTTCAAATGC | |
| | GCAGCTCTTAGTC | |
| 12 | GGGTGGGGCCCGCGCGTATAAAGGGGGCGCAGGCGGGCTGGGC | 100 bp |
| | GTTCCACAGGCCAAGTGCGCTGTGCTCGAGGGGTGCCGGCCAG | |
| | GCCTGAGCGAGCGA | |
| 13 | GGTGCGATATTCGGATTGGCTGGAGTCGGCCATCACGCTCCAGC | 100 bp |
| | TACGCCACTTCCTTTTCGTGGCACTATAAAGGGTGCTGCACGGC | |
| | GCTTGCATCTCT | |
| 14 | ACTTCCGGGTCAGGTGGGCCGGCTGTCTTGACCTTCTTTGCGGC | 100 bp |
| | TCGGCCATTTTGTCCCAGTCAGTCCGGAGGCTGCGGCTGCAGAA | |
| | GTACCGCCTGCG | |
| 15 | GCTGAGCGCGCGCGATGGGGCGGGAGGTTTGGGGTCAAGGAGC | 100 bp |
| | AAACTCTGCACAAGATGGCGGCGGTAGCGGCAGTGGCGGCGCG | |
| | TAGGAGGCGGTGAG | |
| 16 | ATTTTCATCTCGCGAGACTTGTGAGCGGCCATCTTGGTCCTGCC | 100 bp |
| | CTGACAGATTCTCCTATCGGGGTCACAGGGACGCTAAGATTGCT | |
| | ACCTGGACTTTC | |
| 17 | TGGGACCCCCGGAAGGCGGAAGTTCTAGGGCGGAAGTGGCCGA | 100 bp |
| | GAGGAGAGGAGAATGGCGGCGGAAGGCTGGATTTGGCGTTGGG | |
| | GCTGGGGCCGGCGG | |
| 18 | AAGGCCCCTCGGTCTAAGGCTTCCCTATTTCCTGGTTCGCCGGC | 100 bp |
| | GGCCATTTTGGGTGGAAGCGATAGCTGAGTGGCGGCGGCTGCT | |
| | GATTGTGTTCTAG | |
| 19 | AGTGACCCGGAAGTAGAAGTGGCCCTTGCAGGCAAGAGTGCTG | 100 bp |
| | GAGGGCGGCAGCGGCGACCGGAGCGGTAGGAGCAGCAATTTAT | |
| | CCGTGTGCAGCCCC | |
| 20 | GGGAGGGGCGCGCTGGGGAGCTTCGGCGCATGCGCGCTGAGGC | 100 bp |
| | CTGCCTGACCGACCTTCAGCAGGGCTGTGGCTACCATGTTCTCT | |
| | CGCGCGGGTGTCG | |
| 21 | ACTGCGCACGCGCGCGGTCGCACCGATTCACGCCCCCTTCCGGC | 100 bp |
| | GCCTAGAGCACCGCTGCCGCCATGTTGAGGGGGGGACCGCGAC | |
| | CAGCTGGGCCCCT | |
| 22 | CCCTCGAGGGGCGGAGCAAAAAGTGAGGCAGCAACGCCTCCTT | 100 bp |
| | ATCCTCGCTCCCGCTTTCAGTTCTCAATAAGGTCCGATGTTCGTG | |
| | TATAAATGCTCG | |
| 23 | CTTGGTGACCAAATTTGAAAAAAAAAAAAAACCGCGCCAACTC | 100 bp |
| | ATGTTGTTTTCAATCAGGTCCGCCAAGTTTGTATTTAAGGAACT | |
| | GTTTCAGTTCATA | |
| 24 | GGCTGAGCTATCCTATTGGCTATCGGGACAAAATTTGCTTGAGC | 100 bp |
| | CAATCAAAGTGCTCCGTGGACAATCGCCGTTCTGTCTATAAAAA | |
| | GGTGAAGCAGCG | |
| 25 | GGAAGTGCCAGACCGGAGGTGCGTCATTCACCGGCGACGCCGA | 100 bp |
| | TACGGTTCCTCCACCGAGGCCCATGCGAAGCTTTCCACTATGGC | |
| | TTCCAGCACTGTC | |
| 26 | CCCTCGAGGGGCGGAGCAAAAAGTGAGGCAGCAACGCCTCCTT | 100 bp |
| | ATCCTCGCTCCCGCTTTCAGTTCTCAATAAGGTCCGATGTTCGTG | |
| | TATAAATGCTCG | |
| 27 | CTTGGTGACCAAATTTGAAAAAAAAAAAAAACCGCGCCAACTC | 100 bp |
| | ATGTTGTTTTCAATCAGGTCCGCCAAGTTTGTATTTAAGGAACT | |
| | GTTTCAGTTCATA | |
| 28 | GGCTGAGCTATCCTATTGGCTATCGGGACAAAATTTGCTTGAGC | 100 bp |
| | CAATCAAAGTGCTCCGTGGACAATCGCCGTTCTGTCTATAAAAA | |
| | GGTGAAGCAGCG | |
| 29 | GGAAGTGCCAGACCGGAGGTGCGTCATTCACCGGCGACGCCGA | 100 bp |
| | TACGGTTCCTCCACCGAGGCCCATGCGAAGCTTTCCACTATGGC | |
| | TTCCAGCACTGTC | |

TABLE 6
Additional nucleic acid sequences disclosed herein

| | | Source / |
|---|---|---|
| SEQ ID | | Genomic |
| NO: | Nucleic Acid Sequence | Location |
| 30 | GTGATGCGGTTTTGGCAGTACATCAATGGGCGTGGATAGCGGTT | CMV |
| | TGACTCACGGGGATTTCCAAGTCTCCACCCCATTGACGTCAATG | Promoter |
| | GGAGTTTGTTTTGGCACCAAAATCAACGGGACTTTCCAAAATGT | |
| | CGTAACAACTCCGCCCCATTGACGCAAATGGGCGGTAGGCGTG | |
| | TACGGTGGGAGGTCTATATAAGCAGAGCT | |
| 31 | TCGAGGTGAGCCCCACGTTCTGCTTCACTCTCCCCATCTCCCCCC | CBA |
| | CCTCCCCACCCCCAATTTTGTATTTATTTATTTTTTAATTATTTTG | Promoter |
| | TGCAGCGATGGGGGCGGGGGGGGGGGGGGGGCGCGCGCCAGG | |
| | CGGGGCGGGGCGGGGCGAGGGGCGGGGCGGGGCGAGGCGGAG | |
| | AGGTGCGGCGGCAGCCAATCAGAGCGGCGCGCTCCGAAAGTTT | |
| | CCTTTTATGGCGAGGCGGCGGCGGCGGCGGCCCTATAAAAAGC | |
| | GAAGCGCGCGGCGGGCG | |
| 32 | GCGTTACATAACTTACGGTAAATGGCCCGCCTGGCTGACCGCCC | CMV |
| | AACGACCCCCGCCCATTGACGTCAATAATGACGTATGTTCCCAT | enhancer |
| | AGTAACGCCAATAGGGACTTTCCATTGACGTCAATGGGTGGAG | used |
| | TATTTACGGTAAACTGCCCACTTGGCAGTACATCAAGTGTATCA | upstream |
| | TATGCCAAGTACGCCCCCTATTGACGTCAATGACGGTAAATGGC | of CBA |
| | CCGCCTGGCATTATGCCCAGTACATGACCTTATGGGACTTTCCT | promoter |
| | ACTTGGCAGTACATCTACGTATTAGTCATCGCTATTACCATG | |
| 33 | GTACTTATATAAGGGGGTGGGGGCGCGTTCGTCCTCAGTCGCGA | SCP |
| | TCGAACACTCGAGCCGAGCAGACGTGCCTACGGACC | |
| 34 | GGGGAGGCTGCTGGTGAATATTAACCAAGGTCACCCCAGTTAT | SerpE_ |
| | CGGAGGAGCAAACAGGGGCTAAGTCCACGCTAGCGTCTGTCTG | TTR |
| | CACATTTCGTAGAGCGAGTGTTCCGATACTCTAATCTCCCTAGG | |
| | CAAGGTTCATATTTGTGTAGGTTACTTATTCTCCTTTTGTTGACT | |
| | AAGTCAATAATCAGAATCAGCAGGTTTGGAGTCAGCTTGGCAG | |
| | GGATCAGCAGCCTGGGTTGGAAGGAGGGGGTATAAAAGCCCCT | |
| | TCACCAGGAGAAGCCGTC | |
| 35 | GTTTGCTGCTTGCAATGTTTGCCCATTTTAGGGTGGACACAGGA | Proto1 |
| | CGCTGTGGTTTCTGAGCCAGGGCTAGCGGGCGACTCAGATCCCA | |
| | GCCAGTGGACTTAGCCCCTGTTTGCTCCTCCGATAACTGGGGTG | |
| | ACCTTGGTTAATATTCACCAGCAGCCTCCCCCGTTGCCCCTCTG | |
| | GATCCACTGCTTAAATACGGACGAGGACAGGGCCCTGTCTCCTC | |
| | AGCTTCAGGCACCACCACTGACCTGGGACAGTGAATCGCCAC | |
| 36 | TGATGCGGTTTTGGCAGTACATCAATGGGCGTGGATAGCGGTTT | minCMV |
| | GACTCACGGGGATTTCCAAGTCTCCACCCCATTGACGTCAATGG | |
| | GAGTTTGTTTTGGCACCAAAATCAACGGGACTTTCCAAAATGTC | |
| | GTAACAACTCCGCCCCATTGACGCAAATGGGCGGTAGGCGTGT | |
| | ACGGTGGGAGGTCTATATAAGCAGAGCT | |
| 37 | GTTTGCTGCTTGCAATGTTTGCCCATTTTAGGGTGGACACAGGA | UCL-HLP |
| | CGCTGTGGTTTCTGAGCCAGGGGGCGACTCAGATCCCAGCCAGT | |
| | GGACTTAGCCCCTGTTTGCTCCTCCGATAACTGGGGTGACCTTG | |
| | GTTAATATTCACCAGCAGCCTCCCCCGTTGCCCCTCTGGATCCA | |
| | CTGCTTAAATACGGACGAGGACAGGGCCCTGTCTCCTCAGCTTC | |
| | AGGCACCACCACTGACCTGGGACAGTGAATC | |
| 38 | CGTTACATAACTTACGGTAAATGGCCCGCCTGGCTGACCGCCCA | CMVe |
| | ACGACCCCCGCCCATTGACGTCAATAATGACGTATGTTCCCATA | |
| | GTAACGCCAATAGGGACTTTCCATTGACGTCAATGGGTGGAGT | |
| | ATTTACGGTAAACTGCCCACTTGGCAGTACATCAAGTGTATCAT | |
| | ATGCCAAGTACGCCCCCTATTGACGTCAATGACGGTAAATGGCC | |
| | CGCCTGGCATTATGCCCAGTACATGACCTTATGGGACTTTCCTA | |
| | CTTGGCAGTACATCTACGTATTAGTCATCGCTATTACCATG | |
| 39 | GTTACATAACTTACGGTAAATGGCCCGCCTGGCTGACCGCCCAA | CAG |
| | CGACCCCCGCCCATTGACGTCAATAATGACGTATGTTCCCATAG | |
| | TAACGCCAATAGGGACTTTCCATTGACGTCAATGGGTGGAGTAT | |
| | TTACGGTAAACTGCCCACTTGGCAGTACATCAAGTGTATCATAT | |
| | GCCAAGTACGCCCCCTATTGACGTCAATGACGGTAAATGGCCC | |
| | GCCTGGCATTATGCCCAGTACATGACCTTATGGGACTTTCCTAC | |
| | TTGGCAGTACATCTACGTATTAGTCATCGCTATTACCATGGGTC | |
| | GAGGTGAGCCCCACGTTCTGCTTCACTCTCCCCATCTCCCCCCC | |
| | CTCCCCACCCCCAATTTTGTATTTATTTATTTTTTAATTATTTTGT | |
| | GCAGCGATGGGGGCGGGGGGGGGGGGGGCGCGCGCCAGGCGG | |
| | GGCGGGGCGGGGCGAGGGGCGGGGCGGGGCGAGGCGGAGAGG | |
| | TGCGGCGGCAGCCAATCAGAGCGGCGCGCTCCGAAAGTTTCCT | |
| | TTTATGGCGAGGCGGCGGCGGCGGCGGCCCTATAAAAAGCGAA | |
| | GCGCGCGGCGGGCGGGAGTCGCTGCGTTGCCTTCGCCCCGTGCC | |
| | CCGCTCCGCGCCGCCTCGCGCCGCCCGCCCCGGCTCTGACTGAC | |
| | CGCGTTACTCCCACAGGTGAGCGGGCGGGACGGCCCTTCTCCTC | |
| | CGGGCTGTAATTAGCGCTTGGTTTAATGACGGCTCGTTTCTTTTC | |
| | TGTGGCTGCGTGAAAGCCTTAAAGGGCTCCGGGAGGGCCCTTT | |
| | GTGCGGGGGGGAGCGGCTCGGGGGGTGCGTGCGTGTGTGTGTG | |
| | CGTGGGGAGCGCCGCGTGCGGCCCGCGCTGCCCGGCGGCTGTG | |
| | AGCGCTGCGGGCGCGGCGCGGGGCTTTGTGCGCTCCGCGTGTG | |
| | CGCGAGGGGAGCGCGGCCGGGGGCGGTGCCCCGCGGTGCGGG | |
| | GGGGCTGCGAGGGGAACAAAGGCTGCGTGCGGGGTGTGTGCGT | |
| | GGGGGGGTGAGCAGGGGGTGTGGGCGCGGCGGTCGGGCTGTAA | |
| | CCCCCCCCTGCACCCCCCTCCCCGAGTTGCTGAGCACGGCCCGG | |
| | CTTCGGGTGCGGGGCTCCGTGCGGGGCGTGGCGCGGGGCTCGC | |
| | CGTGCCGGGCGGGGGGTGGCGGCAGGTGGGGGTGCCGGGCGG | |
| | GGCGGGGCCGCCTCGGGCCGGGGAGGGCTCGGGGGAGGGGCG | |
| | CGGCGGCCCCGGAGCGCCGGCGGCTGTCGAGGCGCGGCGAGCC | |
| | GCAGCCATTGCCTTTTATGGTAATCGTGCGAGAGGGCGCAGGG | |
| | ACTTCCTTTGTCCCAAATCTGGCGGAGCCGAAATCTGGGAGGCG | |
| | CCGCCGCACCCCCTCTAGCGGGCGCGGGCGAAGCGGTGCGGCG | |
| | CCGGCAGGAAGGAAATGGGCGGGGAGGGCCTTCGTGCGTCGCC | |
| | GCGCCGCCGTCCCCTTCTCCATCTCCAGCCTCGGGGCTGCCGCA | |
| | GGGGGACGGCTGCCTTCGGGGGGGACGGGGCAGGGCGGGGTTC | |
| | GGCTTCTGGCGTGTGACCGGCGGCTCTAGAGCCTCTGCTAACCA | |
| | TGTTCATGCCTTCTTCTTTTTCCTACAGCTCCTGGGCAACGTGCT | |
| | GGTTGTTGTGCTGTCTCATCATTTTGGCAAAGAATT | |
| 40 | GCTCCGGTGCCCGTCAGTGGGCAGAGCGCACATCGCCCACAGT | EFS |
| | CCCCGAGAAGTTGGGGGGAGGGGTCGGCAATTGAACCGGTGCC | |
| | TAGAGAAGGTGGCGCGGGGTAAACTGGGAAAGTGATGTCGTGT | |
| | ACTGGCTCCGCCTTTTTCCCGAGGGTGGGGGAGAACCGTATATA | |
| | AGTGCAGTAGTCGCCGTGAACGTTCTTTTTCGCAACGGGTTTGC | |
| | CGCCAGAACACAGG | |

TABLE 7
List of additional nucleic acid sequences disclosed herein.

| | | Source / |
|---|---|---|
| SEQ ID | | Genomic |
| NO: | Nucleic Acid Sequence | Location |
| 41 | GGAGGAAGCCATCAACTAAACTACAATGACTGTAAGATACAAA | Human; |
| | ATTGGGAATGGTAACATATTTTGAAGTTCTGTTGACATAAAGAA | hg19: chr2: |
| | TCATGATATTAATGCCCATGGAAATGAAAGGGCGATCAACACT | 171621900- |
| | ATGGTTTGAAAAGGGGGAAATTGTAGAGCACAGATGTGTTCGT | 171622580 |
| | GTGGCAGTGTGCTGTCTCTAGCAATACTCAGAGAAGAGAGAGA | |
| | ACAATGAAATTCTGATTGGCCCCAGTGTGAGCCCAGATGAGGTT | |
| | CAGCTGCCAACTTTCTCTTTCACATCTTATGAAAGTCATTTAAGC | |
| | ACAACTAACTTTTTTTTTTTTTTTTTTTTTTTGAGACAGAGTCTTG | |
| | CTCTGTTGCCCAGGACAGAGTGCAGTAGTGACTCAATCTCGGCT | |
| | CACTGCAGCCTCCACCTCCTAGGCTCAAACGGTCCTCCTGCATC | |
| | AGCCTCCCAAGTAGCTGGAATTACAGGAGTGGCCCACCATGCC | |
| | CAGCTAATTTTTGTATTTTTAATAGATACGGGGGTTTCACCATAT | |
| | CACCCAGGCTGGTCTCGAACTCCTGGCCTCAAGTGATCCACCTG | |
| | CCTCGGCCTCCCAAAGTGCTGGGATTATAGGCGTCAGCCACTAT | |
| | GCCCAACCCGACCAACCTTTTTTAAAATAAATATTTAAAAAATT | |
| | GGTATTTCACATATATACTAGT | |
| 42 | AGTTTGGACAAGAACTATAGTTCTAGCTTTCTCTGGGTCTCCAC | Mouse; |
| | CTTGCAGAGAATGCAGCTTTCATTATCTCATGAGCCAAACTCTC | mm10: chr2: |
| | ATCATCTCTTTCCATATATCTGTCGGTGCTCTTCCATGAGTACTC | 36053858- |
| | TAACACACACAGAAGGAGCACTTACACAGGCTGTTGTTTTTCTC | 36054359 |
| | TTATTATCATAGCTGTTGTTCAGACATGTGCATTCTGTTCTTGTT | |
| | GCTTCAATGCTAAAGGAGTCTCAGGATATGAGAACTGTACCAG | |
| | CCGAGGCATCAGGAAACATGGGTGGAAATTCCCACAGTACTAT | |
| | TTGTTCACTGTGTGACCTTGGGCCAGTCACATCCCTTTCCTGAG | |
| | GCTTCGATTCCCCAAGCTATAAAAGAAGCATCTCTTAACCTTTT | |
| | TTTAGGTCATGAGTCAGGCCCAGCACACTCTCAGGGAGACTCAT | |
| | GAGAGTACAGATCATTTCCCATAGAAAAACCATAGTTTTATATC | |
| | CAGAGGCTTTTCTGTAAG | |
| 43 | GGTTCCAGTTCAGAGGCAGAGCATTTGGGGTTCCCAGTCAGGA | Mouse; |
| | GCTTTCCTCTCTCCGCTCCTTAGTTTCCTCTCTTTAAAAAAAAAT | chr2: |
| | GGGTGATAGTATAGAAAGGAAGCTCTGGGCTCGGGGACCAGGG | 36,091,144- |
| | CCCTGGGATCCCCGCTCCCAGCCACTCGCTCCTGACCCTTCCAG | 36,091,966 |
| | GGACAAGCTCCCCCCCACCCCGTCCTTTCCAGGCTGCCACTAGA | |
| | AGAGATGGGGACGCGTGGTCAGCCGCTTCTGTCGCCCCCCAGG | |
| | GAACGGTCTCACGCTGGAGGGGGCAGTGCCCTCGGAACAGGAC | |
| | AGTCAGCCCAAGCCAGCCAAGCGCGCGCGGACGTCCTTCACCG | |
| | CAGAGCAATTGCAGGTACCCCGGGCAAGCCCCGAAGCGTGTGG | |
| | GCGGGGCTTCGGAGTGGGCGTGGTTGTTCGGGACTTGTGACTCC | |
| | GCCCCTTGTGCGGGGACCCGCGTGAGGCCGCTCCAAGGATGAA | |
| | GCTGCCTGGGGCGTGGCCTCGGACCCTGAGCCTCTGATTGGGCG | |
| | GAGGTCTCAGGGCCCTTCTGCGCCCCACAGGTTATGCAGGCGCA | |
| | GTTCGCGCAGGACAACAACCCGGACGCGCAGACGCTGCAGAAG | |
| | CTGGCGGACATGACGGGCCTCAGTCGCAGGGTCATCCAGGTGG | |
| | GGCTCCGGGGTCTCGGCCTTCAGGTCTAGGGTGAACCTTAGGGA | |
| | AGCGCTGAAGCTCGTAGTGGTACGGATGGTCGCGCGTGCACGT | |
| | GGCCGCCCCTCTCCAGTGTGGCCTAAGGACCCCAGTCGGCACG | |
| | GGTTGACCCTTTTCCTTGATTACTGAGAGTGCAGAGGCTGT | |
| 44 | TGGTGGGAAGACATGTCCAGGGAAGAAATGGCCTCCAGAGGCC | Mouse; |
| | TGAGGTGGGGAAATGCTGGAGGTGGAGAGAGGAACAACTGACT | chr2: |
| | GAAAATGAGCTTCCACTGTGGCTTAGTAGCCTATACCAAGTCTA | 36,095,396- |
| | GAGTATAGGGTAGGAGAAGATTAGGAAAGCGATGGGTCTGAGA | 36,096,028 |
| | ATGATGTGGCCTGTTGACTTTTGTAAACCCAAAGCACCTTGGAC | |
| | TAAACCCTATGAACAGTGTGGTGCCACCAAAGACTATAATGAG | |
| | CTCAGGGAACAGAATTCTGTGTGCATGGTGATTTTTTTTTTTTTT | |
| | TTCTGCTAACTGCAGTCTGGGTGATGCATTGACAAACCAATCCT | |
| | GGAAAGTAAGAGGCAAGGGCAGCTGGGACGGTGAGAGGAGCC | |
| | TGATGGGAACCAGGCCAAGCAGGGCAGCAGAGGCGATGAAGA | |
| | GGATGTGGTGCATCCAGAGACTCACTTCATTAGCTGGAGGCACT | |
| | GCTGGATAGGGTCTGAAGGTTCTGGTATCTGAGTTGGCGGGCTG | |
| | GGTGAGTGGTGGCTCTGCTTCCTGAACAGTGTGTGCAAGAGGA | |
| | AACAGGGTTAAGGGCTAGGACAGTCACAGGTGAGTCAGCCTCA | |
| | CAAGAGCAACCTTCCCCTAGTGCAGA | |
| 45 | GGAGGTCTCCTTTTGCCCCGGTTCCAACAAGAGAATGCAAGGCT | Mouse; |
| | GTATCTCAATTTCCTTGAGCCTCTCTGTATTATAGAAGAAAAGT | mm10: chr2: |
| | AGGGAAGCCATACGCCCCTTCTGAGCTTCAGTGTCTCTCTGTCT | 36102524- |
| | CTGCAAATGAGGCTGGGGAGGCTGGGGGCGGGCGTGAAAGAG | 36103193 |
| | GCCCGCGCCAAGCCGACCCCCACCTCTGCCCCCTCCCCAGGTCA | |
| | ACAACCTCATCTGGCACGTGCGGTGCCTCGAGTGCTCCGTGTGT | |
| | CGCACATCGCTGAGGCAGCAGAATAGCTGCTACATCAAGAACA | |
| | AGGAGATCTACTGCAAGATGGACTACTTCAGGTAGGCAGCGGC | |
| | CATCCCGCCAGCAAGCGCTGGAGCATGAACGCCTTGCACACGC | |
| | GTGCCTAGGCCACTTGTGTGGCCTGTGCTCTCCAATTCCTGAGC | |
| | CCTGCTGTTCAGAGTGCACAACGCGGCTCAGCGCACTGGCCCG | |
| | GCCCTCCTACTCAGCACGTCTTACACAGAAGGGAGCGCCAGTCT | |
| | CAGCCTGAGTTCTGGCGGGGGATCTGCCTCGGGTTCCTCCGATC | |
| | TGACAGGCGCTGGCCACGGGTCTGGTTCCATCTCTGGTCTTTTC | |
| | TGGCCCCGAGCACCAGTGTGTTCTGTTGAGCTCTGATGTCCGAG | |
| | GCTCTGGCCCGGATCA | |
| 46 | CTCTGGCTACCTCTTATCTTGGGCATTCACGACAATTTCTAATTG | Mouse; |
| | CAGGTAGTTTGTGTGTGTGCGCGTGTTTTTTTTCCCCCTCAGAGG | mm10: chr2: |
| | CTTGGATTGCAAAGGAACTAAGCGATTACTTCAAGAGCCACGG | 36103286- |
| | GTTAAGTGCAGGGAGAGGGGGAGAGAGAGGGAAAAAAACCCA | 36104328 |
| | ATCCAAATTCAAATTGCTTCATTAGAGAGACACCGCTTTTGTGG | |
| | GGAAGGGCTTTAAATGCCCACTACAAAGTTAGGACTCATTGTTC | |
| | AGCGCCGGTTTATATAACAGGCGAGGGGAGGCGCTGGGCTCTG | |
| | ACAGCTCCGAGCCAGTTCAGCAGCCGCCGTCGCCTGCATTCCCT | |
| | CCCCCTCCCCCAGGTGATGGCCCAGCCAGGGTCCGGCTGCAAA | |
| | GCGACCACCCGCTGTCTCGAAGGGACCGCTCCGCCTGCCATGGT | |
| | GAGTCCTTTCGGTCCTGCTTTCGGCCCCGAGTCCCCCCAACAGC | |
| | ACAGGCCAGGGCTTCTGGCTCAGCCTTCCGGCTACCAACCTCTA | |
| | CCCCTGCGCTGGAAAACTGCCGATAGGAGCCGCCTCTCGTTGAG | |
| | CCTTGGTTTTTCTGGCCTGGAATGTGAGCTTTGGCTGCTTCCTGC | |
| | ACCCAGGATGCGCTGTGTTAAAAGTTGGGGGCCGTCCCTTCTTC | |
| | TCCAATAGGTCCTTTCATTCTTGTACTCCAGCCTAGGGCGCGAC | |
| | ATCCCTGGCACATTTCGGTGTCAGTCGGTGCGCGAGGAAACCA | |
| | GATTCAACTCTGAGTACTCGGCTAAGCGCTTCGCTGTTCCTCTCT | |
| | CCCATTTCAGGCTCAGTCAGACGCAGAGGCCTTGGCAGGCGCTC | |
| | TGGACAAGGACGAAGGTAGAGCCTCCCCATGTACGCCCAGCAC | |
| | ACCGTCTGTCTGCTCGCCGCCCTCTGCTGCCTCTTCCGTGCCGTC | |
| | TGCCGGCAAGAATATCTGCTCCAGTTGCGGTCTGGAGATCCTGG | |
| | ACCGGTATCTGCTCAAGGTGAGTCAGGGTAGGTGTGCCTGCTTG | |
| | CCCACGGGTGTGGTTTGCAGCCCCAAGAGCTGT | |
| 47 | CAAGACTTTTAAAAGTTTAGATAAATAAACAAACATTTGACGGC | Mouse; |
| | TTTCCATCACATCTAGACTATAATCCAAAGATCTATATGGTCCC | mm10: chr2: |
| | AAACGACTTACACTTAACTACCGTCTCCCATATGGCTTCTTCCC | 36114311- |
| | CCATCAGTCATTGTCCTCAGCCATAGTGGCCTCCCTGTTCCTTTG | 36114817 |
| | GGTACAAGGGAACAACTCCCTGAGAGGTTCCATTAGCTGCTGTT | |
| | GCCTGAGATGCTCTTGAGCCCACACCATCTGCTCATTTCTCTCCT | |
| | CACGTGTCAGTGATTAAGAGGCTGTCCTTGGCCTCCCGTCAAAA | |
| | TTACATCCCTGCCGCTTTCCACTTCTTGCCTTCTTATTTTCTAAAT | |
| | AGAACTAACTCACCACTACCCAACATTCTATATAATTGGATATC | |
| | TGTCCTCTGTTTAAATATAATGTTGACTTCAAGAAAGAACGTTG | |
| | TCACTGCCCTGTCACCAGACTTTTAAACAGTGCCTATCGTGTGG | |
| | CACATGCTCAGTGAAATTG | |
| 48 | TCAACAGGGGGACACTTGGGAAAGAAGGATGGGGACAGAGCC | Mouse; |
| | GAGAGGACTGTTACACATTAGAGAAACATCAGTGACTGTGCCA | mm10: chr15: |
| | GCTTTGGGGTAGACTGCACAAAAGCCCTGAGGCAGCACAGGCA | 78179109- |
| | GGATCCAGTCTGCTGGTCCCAGGAAGCTAACCGTCTCAGACAG | 78179610 |
| | AGCACAAAGCACCGAGACATGTGCCACAAGGCTTGTGTAGAGA | |
| | GGTCAGAGGACAGCGTACAGGTCCCAGAGATCAAACTCAACCT | |
| | CACCAGGCTTGGCAGCAAGCCTTTACCAACCCACCCCCACCCCA | |
| | CCCACCCTGCACGCGCCCCTCTCCCCTCCCCATGGTCTCCCATG | |
| | GCTATCTCACTTGGCCCTAAAATGTTTAAGGATGACACTGGCTG | |
| | CTGAGTGGAAATGAGACAGCAGAAGTCAACAGTAGATTTTAGG | |
| | AAAGCCAGAGAAAAAGGCTTGTGCTGTTTTTAGAAAGCCAAGG | |
| | GACAAGCTAAGATAGGGCCCAAGTAAT | |
| 49 | AAATAGAACTGTGAGATAGGGGGAGAGGGGGCAGGAAGGACA | Mouse; |
| | AGAGACCCCTGTCTCATTGTGATCCCCACCTGTCTGCTCTGTGG | mm10: chr15: |
| | GAGGGTACCCATGAGGGCCAGCCCACAGCCCTTAGGTGGACAT | 78195347- |
| | TGTCTGGTCCTGTCTCACTGTCCCTCCCAGCAGCCCCAGAGGCC | 78196134 |
| | AGGAGACAGGGGTCTCAGTCCTCACTGAGAGATGTGTAAACTG | |
| | AGGCCCAGTGAATGTTGAGGGCCAGGGCATGCCCTTGGTGGGA | |
| | TGTGACCTGGGTCTCCTTCGCACGGGCTTCCTCCCCGAAGCCGA | |
| | GCTGAGCATTTGGAGTTTGAAATGTTTCCGTACTTAGCAATCTG | |
| | CTCCTCTATTCCCGGGCGGACTTCCGATAGCTCCGGCCTTATGC | |
| | TGCACTAGATAAGATGGAGCAGGGAGAGGACACGGCACTACTT | |
| | ATGTAACCGGCCTCTTGAAAAATGGAGCAGCGGTCAGGGCGGA | |
| | ACAAGACGTCCTCTCTCTACGCATCCCTCTCCTTTCCCTGCTAAG | |
| | GCTGCAGCTGGAGTCAGAGGCAGGGCTGTTCCAATCTGTCTTTG | |
| | ATCAGTAACGCAGCCAGCCTCCAGCCTCCGTCAGCCTCCTCATG | |
| | GCTGAGACCCGGCCTCAGTTTCCCCCACTTACATCCCGAGGATC | |
| | AGAGCCTGTGAGGATGAAATGGGATAAGGTAGCTGGAACCGTC | |
| | TGGCAGAGAGCGAGTCCTCAGGACTGTTGATGCCTGTGGCTGCC | |
| | TGGCTTGACCCCAAGTGACCCCGCCTCCTCATCCTGCAGCAGGA | |
| | GAA | |
| 50 | TCTATAGAATGTGTCCCCAGCCTTGTTTTCCACACTTGATACGC | Mouse; |
| | AAGGAATGCATACCACAGAGAGGGATGAGGGTAGCATCCAGCC | mm10: chr15: |
| | TGCTTCCTGTGTGTCGGGGCGCTACAGCCACATCTCCCCAGTCC | 78196305- |
| | ATCTCAGACCGTCACAGAGCTTCGCCGAATGTATAGCTTTGTTC | 78196806 |
| | TCTGTGCAGACAGGGAGACAGAGCCTTGGGAAGCATAGGTGCT | |
| | TGCTTCTTTGCCCACTGAGTCTTAGCTGGACTTGCACACCACAT | |
| | GCCTCACAGCCGGGCGCACTTGCATTTGTCACCCAGGCCCAGTG | |
| | ATGATGGCTCTGCTTGCTTTGTGCTTTGTGCCAACTACAGCTCCA | |
| | GCACCTGTGCCCTGGGTTTTCACTCCTTTAGTTGAACACGTAGTT | |
| | ACTGGGGTTGTAGGGATGGAGCCTTTCTGCTTCCTTCTGGCAAA | |
| | GTCCTTAGCGGCCTGCTGCGGGGGTGGGGGGTGTTCAGGGGAG | |
| | TGGTGATGAAGTATGACAG | |
| 51 | TCTCCAGTTGGAGAAACAGATGCTGTAACTGGGGCCACAGTAT | Mouse; |
| | AAAGAGAGCCCAGACATTGAACTGTCAACACAGAAGCCTGGCA | mm10: chr15: |
| | CACTGGAACTGGCAGTCCAGCTGGGAACAAGGGGTAGAGGCTG | 78205234- |
| | AGGCCACTAAGTCAACTGAGGCAGGAGACATAGGAGCTAAAGC | 78205766 |
| | AGCTGAAGGGTGCAGGACAGCTGGGGGGTCTGAAGTGGGCCTC | |
| | ATGCCCAGAGCTATGAAGTCAGGGGCTGTAGCCTAGGAGCCTT | |
| | GGAAGCCAGCTGGCAAGCTGTGGCCCAAAGACGCTGACTCACC | |
| | AGGAGGGGGCAGCTGGAGCCAGGCACTCCTAAGGTTTCCAGGA | |
| | AGGGCAGCCTTCCAGGGCTCAGCTAGGGGAGACAGTGTTGACA | |
| | GCAAGTTGTCAGGCAACTTGAGCTACTGGGCAGCTGGGAAGCT | |
| | GTCCCTTGGTCCCCAGTATCATCATCACCCCAGACGCTGCCCAC | |
| | CTGCCTCAGGTCCCACACAGTGATCCTCCCATCTTTAACACAAC | |
| | ACATGACCAGAGAGA | |
| 52 | GTCACCCTCCCCCCAAACAACCCCTTCTTCTCTGGTTCGAGAAA | Mouse; |
| | TTACAGGCATGAAAGATATAAATCGGGATGCTTGACTTGGGAA | mm10: chr15: |
| | TATAAATCACTAAAGCTTGGGGGCAGGGGTGGGCGACCTTTGT | 78224841- |
| | GACCGTCCTTGTGCGTGCCAGTAAATCCTGTGGTCCAGGGGAGA | 78225364 |
| | AGAAAAGGCTGTGTGGCTTCTGCTCACAAAGCTGCAGAAACCA | |
| | TTCTTTAAGCCCAAAAGCACTTCCAGAGAGAGCAGAGCATCCC | |
| | CAGGCTGCTGGCTCAGCAAGTTCACTGTGCTCAATCTCAGGAAG | |
| | TGAGGATAAGAGCAGTGCCTGGAGAGTGCCTGGTGCTGAGCTG | |
| | AGGGTTTCTGAACACATTAAAGCGGGGAGCATGGACCGGGCCT | |
| | CAGGAGGGGTGTTGAACATCCCTAGGCAGAGGAGTCTAGCTTC | |
| | CTGGGAAAAGATATCAGGTTAAGCACACACATGTCCTCTGGAA | |
| | TAAGATAATCTTTCTGATCACACACTATACACACACAAAAGCCT | |
| | GCTC | |
| 53 | GCCCTCTAGGCCACCTGACCAGGTCCCCTCAGTCCCCCCCTTCC | Mouse; |
| | CACACTCCCACACTCAGCCCCCCTCCCCCCCCCCCGACCCCTGC | mm10: chr15: |
| | AGGATTATCCTGTCTGTGTTCCTGACTCAGCCTGGGAGCCACCT | 78241348- |
| | GGGCAGCAGGGGCCAAGGGTGTCCTAGAAGGGACCTGGAGTCC | 78241856 |
| | ACGCTGGGCCAAGCCTGCCCTTTCTCCCTCTGTCTTCCGTCCCTG | |
| | CTTGCGGTTCTGCTGAATGTGGTTATTTCTCTGGCTCCTTTTACA | |
| | GAGAATGCTGCTGCTAATTTTATGTGGAGCTCTGAGGCAGTGTA | |
| | ATTGGAAGCCAGACACCCTGTCAGCAGTGGGCTCCCGTCCTGA | |
| | GCTGCCATGCTTCCTGCTCTCCTCCCGTCCCGGCTCCTCATTTCA | |
| | TGCAGCCACCTGTCCCAGGGAGAGAGGAGTCACCCAGGCCCCT | |
| | CAGTCCGCCCCTTAAATAAGAAAGCCTCCGTTGCTCGGCACACA | |
| | TACCAAGCAGCCGCTGGTGCAATCT | |
| 54 | GTGTTCTTCCCTTCCCCTTTGGACCCCCGAGACAAGCCAATAAA | Mouse; |
| | ATACTCGGCAGGGTGGCTTCTCTCCTTTTTTTGCCAGTAATAAA | mm10: chr9: |
| | CAGACTCAGAGCAAGTTAAGGGTCTGGTCCAAGGTCATGGCTG | 107340928- |
| | GGATCAGTGACAGAGCCCAGAAGAGAACCTGAGACTTCTTGCT | 107341325 |
| | GAGCCAAGCTGGAGAGGACAGAAAGGAATGCGTCTACTCCATG | |
| | CATGACCCTCTGCCAGCTTTGCTCCTTCCTAAGGGACCATGAAC | |
| | GATATGTGCACACCGCTCATACGTATGTGCACACCTGCAAGAG | |
| | GAGGCATCCCATGTACACCTATGAGACGCACAGAGAAACATAT | |
| | ATGTAGCCATAGGCTAGAAATTCTTTCTCTTTCTAGGTCTGCCCC | |
| | TCTGCA | |
| 55 | GGACCACTCAGTGTACACGGAATGTAGAATTGAGTCTGCCATTG | Mouse; |
| | GTCTTCCCTCAAAGTCTTGGAGGCTTGGGACTGATATTGGGAGC | mm10: chr9: |
| | ATCTGGGCAGAGAAGGCCACAAAGACAGGGTGGTTTTTCTACA | 107349227- |
| | CTGGGACATACTCGTGAGCATGCACAGAGGCGTGTCCCCAACTT | 107350036 |
| | CCCTGTCACCCCTGTCCTCTGCCGGCTAGAGGGGATGCGGGGGT | |
| | GGACATATGCTGCTATTGGGCAGATATCACATGTTAAGAGGTGG | |
| | GGGGGGGCTCAAGAGGCGGAGGGCTAGGAGCATCCCATGGGG | |
| | AGAGGTTCTGGTTTTCTTGCTGCCTCTAGCTGCTATAAATACGTT | |
| | AGCACTTGAGCAACTGGAAAGCTCTGAGTAATTTAGGATGCAC | |
| | AAAGCTGTAATTTAACTCCAGCATCTCAGTGTGCGAGAGCATTA | |
| | AAGATGTAATTAAGATGTTTACACAAAGAGATTGGAGTCTGTG | |
| | ACACTTGGGGTGCAAAACCCCAGGAAGGGACACAATGGGTGAG | |
| | GTGAGGATCTGTGGGAGGCCTGGGGACAGTCACTTGGATCCCA | |
| | GCTATGAGATGGCAGGCCACCCAGCTGTTTCTCCTTGGAAATGT | |
| | TTTGGCCTGGGGGTTGGGGGTGGGGCATCACACTTTGATATGGA | |
| | GATGGGGCAACAAAGCCTGCAATATCTGGGGGTGGAGAGGTCA | |
| | AGTGGATGGAGTCTTTTGAGATCATGTCAGGAAGAGGGCTCGA | |
| | TCCCCCAAAATCATGGTGACATATGGTGTCTCGGGGTTCACAGG | |
| | AGCTATGTCTAAAATACAAAAGTAAA | |
| 56 | TCTGCAGAAGCCTGCCATTCCACCATTTAAACCTGTGACTCCAG | Mouse; |
| | GCCTTAAGCCTGTTGAAGGTCGAGTCCCAGAAGGGTCATATGTG | mm10: chr9: |
| | CAACTGCCTAGGGAGAGTTCCCACTCGCAGGGCCAAGAGGAGT | 107399438- |
| | CCCCCGGTCTGAGGTGTGGGGGCGGGGACGTGCACTGGGCGCT | 107399639 |
| | GGGACCACGGCTGGGGCTCAGGACTCGC | |
| 57 | TGCCTCAGTTTCTTCGCCTAGAAAGCCGGGTCTAAGGGTACATG | Mouse; |
| | CCCTGATTCTTTTCTGGGGTGTCTCGAATTTTAAACAACACATA | mm10: chr9: |
| | CTGTTCTGGGCTGATGACAAGAGGAAGTACTGGTCGGTGGCTG | 107443292- |
| | ATGGACATCCACCATGGTGGCAACTGGAGGGAGGGGGAACGGA | 107444228 |
| | CGTTGAAACCCTGCCCTCCTGGAATCTGTCGCATGCACGCACGT | |
| | TGACAATGCTTGGCACTGGGGACAGGCTGGGATGGATGGAGCG | |
| | GAGCGTGAGGAGGAGTGGGCATGCAGGCCCGAGTGTCTGTTTT | |
| | GCTGATTGCTCCTTTTGCTTTCAAGGAGATTAAACTATTTTTAGT | |
| | CCATGCCTACTGCTGGTGAGACGCTGGAGGAAGCCTTTCCATCG | |
| | TTGAGATTTTCTGGAAGCTGCCAAGTGTGGTCTTCAGCTCAATT | |
| | CTGGGAGCCTCCCAGAGTGGGAGGGAGGAACATTTCCATCTGG | |
| | GGGCTTCGGGGACAGGCTAAGATCTTCCCTGGGGTCCTTGCTGC | |
| | GCTGGCCTCCTCAAACCACGCTGCCTCGGCCTGCATAAAGCAGT | |
| | AATCTGATGTGCCCGATGTTTGTAACGCTGTGTTTAAAAAAAGT | |
| | AATTTATTTTCTAATTATTCCTTGTCTTGCATAACCATGCATTGC | |
| | CAAAGTGTCGCTATTTAAAATATTTATCTCTCCACGCCGCAGGA | |
| | GCAGCTCTGGAGCGTGGAGGGGGAAGAAATAAAAGTCCGCGTG | |
| | CCAGTCGCAGGCATATTACTTTGACTCGTCCTGGTGGCTTTGAC | |
| | GTCTCCCTGTAAATACATTTATTTTTCATTAGGACGTTTCTGAGC | |
| | TTGTGGCCCCCGGAGAGCGGAGTGATTACGCTGTTCATCTGCAA | |
| | GCGATGCAATAGAGGGGTACTCGCAGAATGACTTCCGCCCAGA | |
| | GCATCCTGCGCCTGTCT | |
| 58 | TAAAATACCTTATTTTTTTCCAGTCTCTAAACTGCTAATCTCCCA | Mouse; |
| | GGCTAAGGGATTCTGGGACAAAGGCAAGGCCTGGAAGTGGAAA | mm10: chr9: |
| | TCTGTAAAATTAGCTTCAGCGGTATTAGTGTTTGCAGTTGAAGA | 107444825- |
| | TTGAAAAACTGCTTTCCCAGGGCCTGATTGGAGGCTCCACTCTC | 107445746 |
| | CTCCAGGAAGAGGCAAGGACTCTGGGCTGGCACTGAGGACAAA | |
| | TCCTGGGAGGCTGCTATGGGGCCTGGGAGCCAGGCTGCCTTGTG | |
| | CTAGAGGCCTAGAGAGTGTCTGTGTCCCAAGTCCCAAGCTACCC | |
| | CCAGCAGCTAACAGCTTTTCCAGTTCTCAGGCACAGCAGGTGCC | |
| | AAGATCACGCTCTGGAGTCCAGCTGGGCCCCTTCCTCTTCTTTTT | |
| | TTTTTTTTTTTTTTAAGACCTCCTGGACACTGTTCCTCTCCCCCCC | |
| | CCCGTGACCCCCCCCCTCAGTTCTCAAACACGTGAGGGTTGGGG | |
| | GAGGGTTCCACAGCCAGAGAGAGGGGCCAGCTCTGGTGCCTGT | |
| | GGGTACGCCCGCCCGTATGGCCCATCAGGCCTCTTGTGTGCTTG | |
| | ATTGCCTCTGATTGGCTGCAGCTGAATTCAGCAAAAGCTATTAT | |
| | TTGCCCTTGATGAGCCAATCAGATGGCCTCATTGGCCATTCAGA | |
| | GCAGGCACCGGAACCTGAGGGTGGGGTGGGGGGTGGGGGATG | |
| | GAGATGGGACTCAGTGAGGGGGTGGGAAGCTCTAAAACAGATG | |
| | CAGGACCTGAGCCTGTCTGTGTCCACCACGACCTTCACACAGGT | |
| | CACACCCCCTTCCCCTGACTTGTCACCCCAAACCAGGGCTTGTT | |
| | GCCCAACCCCACCTCACAATTCCCTCACTCTGTAACACCTTTCC | |
| | ATATACCTCTGCATGTCTAAACCCAAGACTTGCTCTATGAAATC | |
| 59 | AGACCCTGCTTAGCACAGCTCTTAGCGGGTCCTTTAGGGGGTCT | Mouse; |
| | CCCAGCGGGCCCAGTGGGAATGAGATAAGGAAGGACACAGCTG | mm10: chr9: |
| | TCCATTCTCCCGTGCCTGCTAAGGAGGAAATGGGGCCGCCTTAC | 107452080- |
| | ATAATTGGGGCAATTTGTTCCACTCTTGTCCTCCTGGTATCATGG | 107452718 |
| | CTATCACCCCCTCCTTGCTCAGGGAGTCCTTGATTGAGCGAGAA | |
| | GCTCAGGCCTCCCTCTCTCCCTCCTGCTGGGGGTTGCTGAACAG | |
| | AGGGTGTAGGAGCCATAGGCTCTGTCACTGCTGAGATCTGCCA | |
| | GATGTCTAGGCCAGGAGAAAATGGAAAGGGCTAAGTCACAGCA | |
| | TATGTGGCCACTCAGGCCTATAGCCCCAAATCTGCCTGGTAACC | |
| | CATTATGTCCCCAGAGAATTTGCATGGGCGGACACCCTCATGCC | |
| | GGGTCTCAGTAAGGGAAGGGGTGGGAGGCAAAAATATCCCTCC | |
| | CCACCCTGAATCTCCACCCCCTCCCCCCAGAAACTGACACTTGG | |
| | CCTTGTCTAAGGATGGGTTTTCCCAAAATCCTTCTGAAAAAAAC | |
| | AGAATTTCAAGAGTCACTCCCTCCGGGTCTCAGCCTAGAACATA | |
| | TGCAGTATCCCCTGACGTCCATAGGG | |
| 60 | AAACTGGCACAGTAATGGCGGGCTGACAGACAAGGGAGTCTGT | Mouse; |
| | AGCACCCGCTGCCTCCGCCCACCCCTTCTCCGAGCAATTAAAAG | mm10: chr9: |
| | GTGTTTATGTGGGGCTGGCAGTGGCTTCTGCCTCCCTTCCATTAC | 107470414- |
| | GAACATTAAGAGATCTTGACCCTTCCACTTTCCCCGCTCTTGAA | 107471129 |
| | AGGAGCTGCAGACACGTGGAGCCAATTAGGCGCACGCGTGGGC | |
| | GCCAAGGGCCTGAGCAGCTTTTTCTCCCTGATTGCGGCGTTTAC | |
| | AGCTGATTATTCTCCCCTCACCCAAACAGTGCTGCTTCCTGGCA | |
| | AGGTGCCACCCAGAGGAGCCGGCTGGGGGCCCCTGGGGACAGG | |
| | GGAGGACTGGATTAGTAAATGGGCATCTATCGAATGGCTTTCAT | |
| | ATGTGTGGCTGGAAGGGAGAAGGGTAGGGCCAGGAATGGTGGC | |
| | AGCAAGGGCCCAGGTAGCAATGAGGGTTCTTCTAACCCACCAT | |
| | TTAGGGATAGCGATCAGAAAAGGGCCCTCGAGGAGGTGACCTA | |
| | AATGTGTGTAGAAGCTGACGGCCACTACACACACACACACACA | |
| | CACACACACACATACACAAGCATCCTTGTCCTTGGAGTCGGTCA | |
| | GCATGAGCAAGAGAAAGATGTTCCCAGTGGCCATGAGAGTGGA | |
| | GCCCTCCTCCCTACTTACATCCAGGTTGGATGGCCAGGAGATCC | |
| | TGAGATCCTTCAAGACTCC | |
| 61 | AAGCCACATCCTGGGTGGAAATATATGGCTTCAATTCCCACTCT | Mouse; |
| | TCCGGATGACCTCTGTGGGGAGCCCTGGCTTCACCTTGGTCCAG | mm10: chr9: |
| | CTTCATCCCTTAGCCTCGCTGCCAGGAAGGCAGTGAGGTCAGAG | 107484887- |
| | GCTGGTGCTGGCGTG | 107485033 |
| 62 | CCTACCTGGTGCCCGCCAACATCTGGGGGCCATCCTGGCCAGCG | Mouse; |
| | CCAGCGTGGTGGTGAAGGCACTGTGCGCCGTGGTACTGTTTCTC | mm10: chr9: |
| | TACCTGCTTTCCTTCGCTGTGGACACGGGCTGCCTGGCCGTCAC | 107534490- |
| | CCCAGGCTACCTTTTCCCACCCAACTTCTGGATCTGGACCCTGG | 107534786 |
| | CCACCCACGGGCTCATGGAACAGCACGTGTGGGACGTGGCCAT | |
| | TAGCCTGGCCACAGTGGTTGTGGCCGGGCGATTACTGGAGCCCC | |
| | TCTGGGGAGCCTTGGAGCTGCTCATCTTCTTCTC | |
| 63 | AAACGGACGGGCCTCCGCTGAACCAGTGAGGCCCCAGACGTGC | Human; |
| | GCATAAATAACCCCTGCGTGCTGCACCACCTGGGGAGAGGGGG | hg19: |
| | AGGACCACGGTAAAT | chr2: |
| | | 171672063- |
| | | 171672163 |
| 64 | GGAGCGAGCGCATAGCAAAAGGGACGCGGGGTCCTTTTCTCTG | Human; |
| | CCGGTGGCACTGGGTAGCTGTGGCCAGGTGTGGTACTTTGATGG | hg19: |
| | GGCCCAGGGCTGGA | chr2: |
| | | 171672697- |
| | | 171672797 |
| 65 | GCTCAAGGAAGCGTCGCAGGGTCACAGATCTGGGGGAACCCCG | Human; |
| | GGGAAAAGCACTGAGGCAAAACCGCCGCTCGTCTCCTACAATA | hg19: |
| | TATGGGAGGGGGAGG | chr2: |
| | | 171672918- |
| | | 171673018 |
| 66 | TTGAGTACGTTCTGGATTACTCATAAGACCTTTTTTTTTTCCTTC | Human; |
| | CGGGCGCAAAACCGTGAGCTGGATTTATAATCGCCCTATAAAG | hg19: |
| | CTCCAGAGGCGGTCAGGCACCTGCAGAGGAGCCCCGCCGCTCC | chr2: |
| | GCCGACTAGCTGCCCCCGCGAGCAACGGCCTCGTGATTTCCCCG | 171673150- |
| | CCGATCCGGTCCCCGCCTCCCCACTCTGCCCCCGCCTACCCCGG | 171673696 |
| | AGCCGTGCAGCCGCCTCTCCGAATCTCTCTCTTCTCCTGGCGCTC | |
| | GCGTGCGAGAGGGAACTAGCGAGAACGAGGAAGCAGCTGGAG | |
| | GTGACGCCGGGCAGATTACGCCTGTCAGGGCCGAGCCGAGCGG | |
| | ATCGCTGGGCGCTGTGCAGAGGAAAGGCGGGAGTGCCCGGCTC | |
| | GCTGTCGCAGAGCCGAGGTGGGTAAGCTAGCGACCACCTGGAC | |
| | TTCCCAGCGCCCAACCGTGGCTTTTCAGCCAGGTCCTCTCCTCC | |
| | CGCGGCTTCTCAACCAACCCCATCCCAGCGCCGGCCACCCAACC | |
| | TCCCGAAATGAGTGCTTCCTGCCC | |
| 67 | CAGCAGCCGAAGGCGCTACTAGGAACGGTAACCTGTTACTTTTC | Human; |
| | CAGGGGCCGTAGTCGACCCGCTGCCCGAGTTGCTGTGCGACTGC | hg19: |
| | GCGCGCGGGGCTA | chr2: |
| | | 171673900- |
| | | 171674000 |
| 68 | GAGTGCAAGGTGACTGTGGTTCTTCTCTGGCCAAGTCCGAGGGA | Human; |
| | GAACGTAAAGATATGGGCCTTTTTCCCCCTCTCACCTTGTCTCA | hg19: |
| | CCAAAGTCCCTAGTCCCCGGAGCAGTTAGCCTCTTTCTTTCCAG | chr2: |
| | GGAATTAGCCAGACACAACAACGGGAACCAGACACCGAACCA | 171674400- |
| | GACATGCCCGCCCCGTGCGCCCTCCCC | 171674600 |
| 69 | GCTCGCTGCCTTTCCTCCCTCTTGTCTCTCCAGAGCCGGATCTTC | Human; |
| | AAGGGGAGCCTCCGTGCCCCCGGCTGCTCAGTCCCTCCGGTGTG | hg19: |
| | CAGGACCCCGGAAGTCCTCCCCGCACAGCTCTCGCTTCTCTTTG | chr2: |
| | CAGCCTGTTTCTGCGCCGGACCAGTCGAGGACTCTGGACAGTAG | 171674903- |
| | AGGCCCCGGGACGACCGAGCTG | 171675101 |
| 70 | AAACGGACGGGCCTCCGCTGAACCAGTGAGGCCCCAGACGTGC | Human |
| | GCATAAATAACCCCTGCGTGCTGCACCACCTGGGGAGAGGGGG | |
| | AGGACCACGGTAAATGGAGCGAGCGCATAGCAAAAGGGACGC | |
| | GGGGTCCTTTTCTCTGCCGGTGGCACTGGGTAGCTGTGGCCAGG | |
| | TGTGGTACTTTGATGGGGCCCAGGGCTGGAGCTCAAGGAAGCG | |
| | TCGCAGGGTCACAGATCTGGGGGAACCCCGGGGAAAAGCACTG | |
| | AGGCAAAACCGCCGCTCGTCTCCTACAATATATGGGAGGGGGA | |
| | GGTTGAGTACGTTCTGGATTACTCATAAGACCTTTTTTTTTTCCT | |
| | TCCGGGCGCAAAACCGTGAGCTGGATTTATAATCGCCCTATAAA | |
| | GCTCCAGAGGCGGTCAGGCACCTGCAGAGGAGCCCCGCCGCTC | |
| | CGCCGACTAGCTGCCCCCGCGAGCAACGGCCTCGTGATTTCCCC | |
| | GCCGATCCGGTCCCCGCCTCCCCACTCTGCCCCCGCCTACCCCG | |
| | GAGCCGTGCAGCCGCCTCTCCGAATCTCTCTCTTCTCCTGGCGC | |
| | TCGCGTGCGAGAGGGAACTAGCGAGAACGAGGAAGCAGCTGG | |
| | AGGTGACGCCGGGCAGATTACGCCTGTCAGGGCCGAGCCGAGC | |
| | GGATCGCTGGGCGCTGTGCAGAGGAAAGGCGGGAGTGCCCGGC | |
| | TCGCTGTCGCAGAGCCGAGGTGGGTAAGCTAGCGACCACCTGG | |
| | ACTTCCCAGCGCCCAACCGTGGCTTTTCAGCCAGGTCCTCTCCT | |
| | CCCGCGGCTTCTCAACCAACCCCATCCCAGCGCCGGCCACCCAA | |
| | CCTCCCGAAATGAGTGCTTCCTGCCCCAGCAGCCGAAGGCGCTA | |
| | CTAGGAACGGTAACCTGTTACTTTTCCAGGGGCCGTAGTCGACC | |
| | CGCTGCCCGAGTTGCTGTGCGACTGCGCGCGCGGGGCTAGAGT | |
| | GCAAGGTGACTGTGGTTCTTCTCTGGCCAAGTCCGAGGGAGAA | |
| | CGTAAAGATATGGGCCTTTTTCCCCCTCTCACCTTGTCTCACCAA | |
| | AGTCCCTAGTCCCCGGAGCAGTTAGCCTCTTTCTTTCCAGGGAA | |
| | TTAGCCAGACACAACAACGGGAACCAGACACCGAACCAGACAT | |
| | GCCCGCCCCGTGCGCCCTCCCCGCTCGCTGCCTTTCCTCCCTCTT | |
| | GTCTCTCCAGAGCCGGATCTTCAAGGGGAGCCTCCGTGCCCCCG | |
| | GCTGCTCAGTCCCTCCGGTGTGCAGGACCCCGGAAGTCCTCCCC | |
| | GCACAGCTCTCGCTTCTCTTTGCAGCCTGTTTCTGCGCCGGACC | |
| | AGTCGAGGACTCTGGACAGTAGAGGCCCCGGGACGACCGAGCT | |
| | G | |
| 71 | GGAGGAAGCCATCAACTAAACTACAATGACTGTAAGATACAAA | Human |
| | ATTGGGAATGGTAACATATTTTGAAGTTCTGTTGACATAAAGAA | |
| | TCATGATATTAATGCCCATGGAAATGAAAGGGCGATCAACACT | |
| | ATGGTTTGAAAAGGGGGAAATTGTAGAGCACAGATGTGTTCGT | |
| | GTGGCAGTGTGCTGTCTCTAGCAATACTCAGAGAAGAGAGAGA | |
| | ACAATGAAATTCTGATTGGCCCCAGTGTGAGCCCAGATGAGGTT | |
| | CAGCTGCCAACTTTCTCTTTCACATCTTATGAAAGTCATTTAAGC | |
| | ACAACTAACTTTTTTTTTTTTTTTTTTTTTTTGAGACAGAGTCTTG | |
| | CTCTGTTGCCCAGGACAGAGTGCAGTAGTGACTCAATCTCGGCT | |
| | CACTGCAGCCTCCACCTCCTAGGCTCAAACGGTCCTCCTGCATC | |
| | AGCCTCCCAAGTAGCTGGAATTACAGGAGTGGCCCACCATGCC | |
| | CAGCTAATTTTTGTATTTTTAATAGATACGGGGGTTTCACCATAT | |
| | CACCCAGGCTGGTCTCGAACTCCTGGCCTCAAGTGATCCACCTG | |
| | CCTCGGCCTCCCAAAGTGCTGGGATTATAGGCGTCAGCCACTAT | |
| | GCCCAACCCGACCAACCTTTTTTAAAATAAATATTTAAAAAATT | |
| | GGTATTTCACATATATACTAGTATTTACATTTATCCACACAAAA | |
| | CGGACGGGCCTCCGCTGAACCAGTGAGGCCCCAGACGTGCGCA | |
| | TAAATAACCCCTGCGTGCTGCACCACCTGGGGAGAGGGGGAGG | |
| | ACCACGGTAAATGGAGCGAGCGCATAGCAAAAGGGACGCGGG | |
| | GTCCTTTTCTCTGCCGGTGGCACTGGGTAGCTGTGGCCAGGTGT | |
| | GGTACTTTGATGGGGCCCAGGGCTGGAGCTCAAGGAAGCGTCG | |
| | CAGGGTCACAGATCTGGGGGAACCCCGGGGAAAAGCACTGAGG | |
| | CAAAACCGCCGCTCGTCTCCTACAATATATGGGAGGGGGAGGT | |
| | TGAGTACGTTCTGGATTACTCATAAGACCTTTTTTTTTTCCTTCC | |
| | GGGCGCAAAACCGTGAGCTGGATTTATAATCGCCCTATAAAGC | |
| | TCCAGAGGCGGTCAGGCACCTGCAGAGGAGCCCCGCCGCTCCG | |
| | CCGACTAGCTGCCCCCGCGAGCAACGGCCTCGTGATTTCCCCGC | |
| | CGATCCGGTCCCCGCCTCCCCACTCTGCCCCCGCCTACCCCGGA | |
| | GCCGTGCAGCCGCCTCTCCGAATCTCTCTCTTCTCCTGGCGCTCG | |
| | CGTGCGAGAGGGAACTAGCGAGAACGAGGAAGCAGCTGGAGG | |
| | TGACGCCGGGCAGATTACGCCTGTCAGGGCCGAGCCGAGCGGA | |
| | TCGCTGGGCGCTGTGCAGAGGAAAGGCGGGAGTGCCCGGCTCG | |
| | CTGTCGCAGAGCCGAGGTGGGTAAGCTAGCGACCACCTGGACT | |
| | TCCCAGCGCCCAACCGTGGCTTTTCAGCCAGGTCCTCTCCTCCC | |
| | GCGGCTTCTCAACCAACCCCATCCCAGCGCCGGCCACCCAACCT | |
| | CCCGAAATGAGTGCTTCCTGCCCCAGCAGCCGAAGGCGCTACT | |
| | AGGAACGGTAACCTGTTACTTTTCCAGGGGCCGTAGTCGACCCG | |
| | CTGCCCGAGTTGCTGTGCGACTGCGCGCGCGGGGCTAGAGTGC | |
| | AAGGTGACTGTGGTTCTTCTCTGGCCAAGTCCGAGGGAGAACGT | |
| | AAAGATATGGGCCTTTTTCCCCCTCTCACCTTGTCTCACCAAAG | |
| | TCCCTAGTCCCCGGAGCAGTTAGCCTCTTTCTTTCCAGGGAATT | |
| | AGCCAGACACAACAACGGGAACCAGACACCGAACCAGACATG | |
| | CCCGCCCCGTGCGCCCTCCCCGCTCGCTGCCTTTCCTCCCTCTTG | |
| | TCTCTCCAGAGCCGGATCTTCAAGGGGAGCCTCCGTGCCCCCGG | |
| | CTGCTCAGTCCCTCCGGTGTGCAGGACCCCGGAAGTCCTCCCCG | |
| | CACAGCTCTCGCTTCTCTTTGCAGCCTGTTTCTGCGCCGGACCA | |
| | GTCGAGGACTCTGGACAGTAGAGGCCCCGGGACGACCGAGCTG | |
| 72 | TCAACAGGGGGACACTTGGGAAAGAAGGATGGGGACAGAGCC | Human and |
| | GAGAGGACTGTTACACATTAGAGAAACATCAGTGACTGTGCCA | mouse |
| | GCTTTGGGGTAGACTGCACAAAAGCCCTGAGGCAGCACAGGCA | |
| | GGATCCAGTCTGCTGGTCCCAGGAAGCTAACCGTCTCAGACAG | |
| | AGCACAAAGCACCGAGACATGTGCCACAAGGCTTGTGTAGAGA | |
| | GGTCAGAGGACAGCGTACAGGTCCCAGAGATCAAACTCAACCT | |
| | CACCAGGCTTGGCAGCAAGCCTTTACCAACCCACCCCCACCCCA | |
| | CCCACCCTGCACGCGCCCCTCTCCCCTCCCCATGGTCTCCCATG | |
| | GCTATCTCACTTGGCCCTAAAATGTTTAAGGATGACACTGGCTG | |
| | CTGAGTGGAAATGAGACAGCAGAAGTCAACAGTAGATTTTAGG | |
| | AAAGCCAGAGAAAAAGGCTTGTGCTGTTTTTAGAAAGCCAAGG | |
| | GACAAGCTAAGATAGGGCCCAAGTAATGCTAGTATTTACATTTA | |
| | TCCACACAAAACGGACGGGCCTCCGCTGAACCAGTGAGGCCCC | |
| | AGACGTGCGCATAAATAACCCCTGCGTGCTGCACCACCTGGGG | |
| | AGAGGGGGAGGACCACGGTAAATGGAGCGAGCGCATAGCAAA | |
| | AGGGACGCGGGGTCCTTTTCTCTGCCGGTGGCACTGGGTAGCTG | |
| | TGGCCAGGTGTGGTACTTTGATGGGGCCCAGGGCTGGAGCTCA | |
| | AGGAAGCGTCGCAGGGTCACAGATCTGGGGGAACCCCGGGGAA | |
| | AAGCACTGAGGCAAAACCGCCGCTCGTCTCCTACAATATATGG | |
| | GAGGGGGAGGTTGAGTACGTTCTGGATTACTCATAAGACCTTTT | |
| | TTTTTTCCTTCCGGGCGCAAAACCGTGAGCTGGATTTATAATCG | |
| | CCCTATAAAGCTCCAGAGGCGGTCAGGCACCTGCAGAGGAGCC | |
| | CCGCCGCTCCGCCGACTAGCTGCCCCCGCGAGCAACGGCCTCGT | |
| | GATTTCCCCGCCGATCCGGTCCCCGCCTCCCCACTCTGCCCCCG | |
| | CCTACCCCGGAGCCGTGCAGCCGCCTCTCCGAATCTCTCTCTTC | |
| | TCCTGGCGCTCGCGTGCGAGAGGGAACTAGCGAGAACGAGGAA | |
| | GCAGCTGGAGGTGACGCCGGGCAGATTACGCCTGTCAGGGCCG | |
| | AGCCGAGCGGATCGCTGGGCGCTGTGCAGAGGAAAGGCGGGA | |
| | GTGCCCGGCTCGCTGTCGCAGAGCCGAGGTGGGTAAGCTAGCG | |
| | ACCACCTGGACTTCCCAGCGCCCAACCGTGGCTTTTCAGCCAGG | |
| | TCCTCTCCTCCCGCGGCTTCTCAACCAACCCCATCCCAGCGCCG | |
| | GCCACCCAACCTCCCGAAATGAGTGCTTCCTGCCCCAGCAGCCG | |
| | AAGGCGCTACTAGGAACGGTAACCTGTTACTTTTCCAGGGGCCG | |
| | TAGTCGACCCGCTGCCCGAGTTGCTGTGCGACTGCGCGCGCGGG | |
| | GCTAGAGTGCAAGGTGACTGTGGTTCTTCTCTGGCCAAGTCCGA | |
| | GGGAGAACGTAAAGATATGGGCCTTTTTCCCCCTCTCACCTTGT | |
| | CTCACCAAAGTCCCTAGTCCCCGGAGCAGTTAGCCTCTTTCTTT | |
| | CCAGGGAATTAGCCAGACACAACAACGGGAACCAGACACCGA | |
| | ACCAGACATGCCCGCCCCGTGCGCCCTCCCCGCTCGCTGCCTTT | |
| | CCTCCCTCTTGTCTCTCCAGAGCCGGATCTTCAAGGGGAGCCTC | |
| | CGTGCCCCCGGCTGCTCAGTCCCTCCGGTGTGCAGGACCCCGGA | |
| | AGTCCTCCCCGCACAGCTCTCGCTTCTCTTTGCAGCCTGTTTCTG | |
| | CGCCGGACCAGTCGAGGACTCTGGACAGTAGAGGCCCCGGGAC | |
| | GACCGAGCTG | |
| 73 | ATTTACATTTATCCACACA | Human |
| 74 | TGCCGCTGGACTCTCTTCCAAGGAACTAGGAGAACCAAGATCC | Mouse; |
| | GTTTTTCTGCCAAGGGCTGCCCCCCCCACGCCCCCAACCCCCTC | chr9: |
| | ACCCCGATCCCCACAGAAAGAAATCTTGAGGTAGCTGGAGCTT | 107,399,268- |
| | CTTCTGTGGGTGTGACAGGACTGCCATTCTCCTCTGTAGTCTGC | 107,400,067 |
| | AGAAGCCTGCCATTCCACCATTTAAACCTGTGACTCCAGGCCTT | |
| | AAGCCTGTTGAAGGTCGAGTCCCAGAAGGGTCATATGTGCAAC | |
| | TGCCTAGGGAGAGTTCCCACTCGCAGGGCCAAGAGGAGTCCCC | |
| | CGGTCTGAGGTGTGGGGGCGGGGACGTGCACTGGGCGCTGGGA | |
| | CCACGGCTGGGGCTCAGGACTCGCGAGCTTGGATTCGGATCGG | |
| | TTTGCGCGAGCCAGTAGGGCAGGCTCCGGGGTGAACGGGGACG | |
| | AGGGGCGCGCGGGCACAGGCGGGCGCGTGACCGCGGCGGGGG | |
| | CGCGCGGAGGCGGGCCGGCCAAGGAGAGGGAGGGAGGGAATG | |
| | AGGGAGGGAGCGACAGGGGAGGGCGGCGCCGGCAGGTTGGCG | |
| | GCGGCCGCTATTTGAGCGCAGGTCCCGGGCCAGGCGCTCAAAG | |
| | CGCTTGGAGCCAGCGCGGCGGGGAGATCGCTGCGCGCAGCCCG | |
| | CAGAGGCGCTGCGGCCAGTGCAGCCCCGGAGGCCCCGCGCGGA | |
| | GAAGGAGGTGGAGAAGAGGCCGGCTTTCCGCCCGCCGCCCGCG | |
| | CCCCCCCACCTCCATCCCGCCGCCGCCGTCCCCCCTCCCTCCCC | |
| | GCGGCGCCGCATCTTGAATGGAAAC | |
| 75 | GAGTAATTCATACAAAAGGACTCGCCCCTGCCTTGGGGAATCCC | |
| | AGGGACCGTCGTTAAACTCCCACTAACGTAGAACCCAGAGATC | |
| | GCTGCGTTCCCGCCCCCTCACCCGCCCGCTCTCGTCATCACTGA | |
| | GGTGGAGAAGAGCATGCGTGAGGCTCCGGTGCCCGTCAGTGGG | |
| | CAGAGCGCACATCGCCCACAGTCCCCGAGAAGTTGGGGGGAGG | |
| | GGTCGGCAATTGAACCGGTGCCTAGAGAAGGTGGCGCGGGGTA | |
| | AACTGGGAAAGTGATGTCGTGTACTGGCTCCGCCTTTTTCCCGA | |
| | GGGTGGGGGAGAACCGTATATAAGTGCAGTAGTCGCCGTGAAC | |
| | GTTCTTTTTCGCAACGGGTTTGCCGCCAGAACACAGGTAAGTGC | |
| | CGTGTGTGGTTCCCGCGGGCCTGGCCTCTTTACGGGTTATGGCC | |
| | CTTGCGTGCCTTGAATTACTTCCACGCCCCTGGCTGCAGTACGT | |
| | GATTCTTGATCCCGAGCTTCGGGTTGGAAGTGGGTGGGAGAGTT | |
| | CGAGGCCTTGCGCTTAAGGAGCCCCTTCGCCTCGTGCTTGAGTT | |
| | GAGGCCTGGCTTGGGCGCTGGGGCCGCCGCGTGCGAATCTGGT | |
| | GGCACCTTCGCGCCTGTCTCGCTGCTTTCGATAAGTCTCTAGCC | |
| | ATTTAAAATTTTTGATGACCTGCTGCGACGCTTTTTTTCTGGCAA | |
| | GATAGTCTTGTAAATGCGGGCCAAGATCTGCACACTGGTATTTC | |
| | GGTTTTTGGGGCCGCGGGCGGCGACGGGGCCCGTGCGTCCCAG | |
| | CGCACATGTTCGGCGAGGCGGGGCCTGCGAGCGCGGCCACCGA | |
| | GAATCGGACGGGGGTAGTCTCAAGCTGGCCGGCCTGCTCTGGT | |
| | GCCTGGCCTCGCGCCGCCGTGTATCGCCCCGCCCTGGGCGGCAA | |
| | GGCTGGCCCGGTCGGCACCAGTTGCGTGAGCGGAAAGATGGCC | |
| | GCTTCCCGGCCCTGCTGCAGGGAGCTCAAAATGGAGGACGCGG | |
| | CGCTCGGGAGAGCGGGCGGGTGAGTCACCCACACAAAGGAAA | |
| | AGGGCCTTTCCGTCCTCAGCCGTCGCTTCATGTGACTCCACGGA | |
| | GTACCGGGCGCCGTCCAGGCACCTCGATTAGTTCTCGAGCTTTT | |
| | GGAGTACGTCGTCTTTAGGTTGGGGGGAGGGGTTTTATGCGATG | |
| | GAGTTTCCCCACACTGAGTGGGTGGAGACTGAAGTTAGGCCAG | |
| | CTTGGCACTTGATGTAATTCTCCTTGGAATTTGCCCTTTTTGAGT | |
| | TTGGATCTTGGTTCATTCTCAAGCCTCAGACAGTGGTTCAAAGT | |
| | TTTTTTCTTCCATTTCAGGTGTCGTGA | |
| 76 | GGAGGAAGCCATCAACTAAACTACAATGACTGTAAGATACAAA | |
| | ATTGGGAATGGTAACATATTTTGAAGTTCTGTTGACATAAAGAA | |
| | TCATGATATTAATGCCCATGGAAATGAAAGGGCGATCAACACT | |
| | ATGGTTTGAAAAGGGGGAAATTGTAGAGCACAGATGTGTTCGT | |
| | GTGGCAGTGTGCTGTCTCTAGCAATACTCAGAGAAGAGAGAGA | |
| | ACAATGAAATTCTGATTGGCCCCAGTGTGAGCCCAGATGAGGTT | |
| | CAGCTGCCAACTTTCTCTTTCACATCTTATGAAAGTCATTTAAGC | |
| | ACAACTAACTTTTTTTTTTTTTTTTTTTTTTTGAGACAGAGTCTTG | |
| | CTCTGTTGCCCAGGACAGAGTGCAGTAGTGACTCAATCTCGGCT | |
| | CACTGCAGCCTCCACCTCCTAGGCTCAAACGGTCCTCCTGCATC | |
| | AGCCTCCCAAGTAGCTGGAATTACAGGAGTGGCCCACCATGCC | |
| | CAGCTAATTTTTGTATTTTTAATAGATACGGGGGTTTCACCATAT | |
| | CACCCAGGCTGGTCTCGAACTCCTGGCCTCAAGTGATCCACCTG | |
| | CCTCGGCCTCCCAAAGTGCTGGGATTATAGGCGTCAGCCACTAT | |
| | GCCCAACCCGACCAACCTTTTTTAAAATAAATATTTAAAAAATT | |
| | GGTATTTCACATATATACTAGTATTTACATTTATCCACACAAAA | |
| | CGGACGGGCCTCCGCTGAACCAGTGAGGCCCCAGACGTGCGCA | |
| | TAAATAACCCCTGCGTGCTGCACCACCTGGGGAGAGGGGGAGG | |
| | ACCACGGTAAATGGAGCGAGCGCATAGCAAAAGGGACGCGGG | |
| | GTCCTTTTCTCTGCCGGTGGCACTGGGTAGCTGTGGCCAGGTGT | |
| | GGTACTTTGATGGGGCCCAGGGCTGGAGCTCAAGGAAGCGTCG | |
| | CAGGGTCACAGATCTGGGGGAACCCCGGGGAAAAGCACTGAGG | |
| | CAAAACCGCCGCTCGTCTCCTACAATATATGGGAGGGGGAGGT | |
| | TGAGTACGTTCTGGATTACTCATAAGACCTTTTTTTTTTCCTTCC | |
| | GGGCGCAAAACCGTGAGCTGGATTTATAATCGCCCTATAAAGC | |
| | TCCAGAGGCGGTCAGGCACCTGCAGAGGAGCCCCGCCGCTCCG | |
| | CCGACTAGCTGCCCCCGCGAGCAACGGCCTCGTGATTTCCCCGC | |
| | CGATCCGGTCCCCGCCTCCCCACTCTGCCCCCGCCTACCCCGGA | |
| | GCCGTGCAGCCGCCTCTCCGAATCTCTCTCTTCTCCTGGCGCTCG | |
| | CGTGCGAGAGGGAACTAGCGAGAACGAGGAAGCAGCTGGAGG | |
| | TGACGCCGGGCAGATTACGCCTGTCAGGGCCGAGCCGAGCGGA | |
| | TCGCTGGGCGCTGTGCAGAGGAAAGGCGGGAGTGCCCGGCTCG | |
| | CTGTCGCAGAGCCGAGGTGGGTAAGCTAGCGACCACCTGGACT | |
| | TCCCAGCGCCCAACCGTGGCTTTTCAGCCAGGTCCTCTCCTCCC | |
| | GCGGCTTCTCAACCAACCCCATCCCAGCGCCGGCCACCCAACCT | |
| | CCCGAAATGAGTGCTTCCTGCCCCAGCAGCCGAAGGCGCTACT | |
| | AGGAACGGTAACCTGTTACTTTTCCAGGGGCCGTAGTCGACCCG | |
| | CTGCCCGAGTTGCTGTGCGACTGCGCGCGCGGGGCTAGAGTGC | |
| | AAGGTGACTGTGGTTCTTCTCTGGCCAAGTCCGAGGGAGAACGT | |
| | AAAGATATGGGCCTTTTTCCCCCTCTCACCTTGTCTCACCAAAG | |
| | TCCCTAGTCCCCGGAGCAGTTAGCCTCTTTCTTTCCAGGGAATT | |
| | AGCCAGACACAACAACGGGAACCAGACACCGAACCAGACATG | |
| | CCCGCCCCGTGCGCCCTCCCCGCTCGCTGCCTTTCCTCCCTCTTG | |
| | TCTCTCCAGAGCCGGATCTTCAAGGGGAGCCTCCGTGCCCCCGG | |
| | CTGCTCAGTCCCTCCGGTGTGCAGGACCCCGGAAGTCCTCCCCG | |
| | CACAGCTCTCGCTTCTCTTTGCAGCCTGTTTCTGCGCCGGACCA | |
| | GTCGAGGACTCTGGACAGTAGAGGCCCCGGGACGACCGAGCTG | |
| 77 | GAATGTGGGAAATCATTCAGTCGC | eTF^{SCN1A} |
| | | Forward |
| | | primer |
| 78 | GCAAGTTATCCTCTCGTGAGAAGG | eTF^{SCN1A} |
| | | Reverse |
| | | primer |
| 79 | GCGACAACCTGGTGAGACATCAACGCACC | eTF^{SCN1A} |
| | | probe |
| 80 | GCTGTTATCTCTTGTGGGCTGT | MfAlb |
| | | Forward |
| | | primer |
| 81 | AAACTCATGGGAGCTGCCGGTT | MfAlb |
| | | Reverse |
| | | primer |
| 82 | CCACACAAATCTCTCCCTGGCATTG | MfAlb |
| | | probe |
| 83 | GAATGTGGGAAATCATTCAGTCGC | eTF^{SCN1A} |
| | | Forward |
| | | primer |
| 84 | GCAAGTTATCCTCTCGTGAGAAGG | eTF^{SCN1A} |
| | | Reverse |
| | | primer |
| 85 | GCGACAACCTGGTGAGACATCAACGCACC | eTF^{SCN1A} |
| | | probe |

### INCORPORATION BY REFERENCE

All publications, patents, and patent applications mentioned in this specification are herein incorporated by reference to the same extent as if each individual publication, patent, or patent application was specifically and individually indicated to be incorporated by reference.

What is claimed is:

1. A method of administering a vector to a primate, comprising intracerebroventricular (ICV) administration of a vector to the primate, wherein the vector comprises a cell-type selective regulatory element.

2. A method of administering a vector to a primate, comprising intracerebroventricular (ICV) administration of a vector to the primate, wherein the vector comprises a regulatory element, wherein the regulatory element results in increased transgene expression by at least 2 fold as compared to expression of the transgene when operably linked to a CMV promoter.

3. A method of administering a vector to a primate, comprising intracerebroventricular (ICV) administration of a vector to the primate, wherein the vector is administered unilaterally.

4. A method of administering a vector to a primate, comprising intracerebroventricular (ICV) administration of a vector to the primate, wherein the vector is not a self-complementary AAV.

5. The method of claim 1, wherein the primate is a human.

6. The method of claim 1, wherein the primate is a non-human primate.

7. The method of claim 6, wherein the non-human primate is an old world monkey, an orangutan, a gorilla, a chimpanzee, a crab-eating macaque, a rhesus macaque or a pig-tailed macaque.

8. The method of any one of claims 3-7, wherein the vector comprises a nucleotide sequence operably linked to a regulatory element.

9. The method of claim 1, 2 or 8, wherein the regulatory element is selectively expressed in neuronal cells.

10. The method of claim 9, wherein the neuronal cells are selected from the group consisting of unipolar, bipolar, multipolar, or pseudounipolar neurons.

11. The method of claim 9, wherein the neuronal cells are GABAergic neurons.

12. The method of claim 2 or 8, wherein the regulatory element is selectively expressed in glial cells.

13. The method of claim 12, wherein the glial cells are selected from the group consisting of astrocytes, oligodendrocytes, ependymal cells, Schwann cells, and satellite cells.

14. The method of claim 2 or 8, wherein the regulatory element is selectively expressed in non-neuronal cells.

15. The method of any one of claims 1-14, wherein the vector is administered to more than one ventricle of the brain.

16. The method of any one of claims claim 1-2 or 4-15, wherein the vector is administered bilaterally.

17. The method of claim 15 or 16, wherein the vector is administered simultaneously.

18. The method of claim 15 or 16, wherein the vector is administered sequentially.

19. The method of claim 18, wherein each dose of the vector is administered at least 24 hours apart.

20. The method of any one of claims 1-14, wherein the vector is administered to one ventricle of the brain.

21. The method of any one of claims 1-20, wherein the primate further receives an intravenous administration of the vector.

22. The method of any one of claims 1-21, wherein the primate further receives an intrathecal administration of the vector.

23. The method of claim 22, wherein the intrathecal administration comprises intrathecal cisternal administration or intrathecal lumbar administration.

24. The method of any one of claims 1-23, wherein the vector comprises a nucleotide sequence encoding a polypeptide.

25. The method of claim 24, wherein the polypeptide is a DNA binding protein.

26. The method of claim 25, wherein the DNA binding protein is selected from the group consisting of a zinc finger protein (ZFP), a zinc finger nuclease (ZFN), or a transcription activator-like effector nuclease (TALEN).

27. The method of any one of claims 24-26, wherein the nucleotide sequence is a codon-optimized variant and/or a fragment thereof.

28. The method of any one of claims 1-23, wherein the vector comprises a nucleotide sequence encoding a guide RNA (gRNA).

29. The method of any one of claims 1-28, wherein the vector comprises a nucleotide sequence encoding an interfering RNA (RNAi) that reduces expression of a target gene.

30. The method of claim 29, wherein the RNAi reduces expression of a target gene selected from the group consisting of SOD1, HTT, Tau, or alpha-synuclein.

31. The method of any one of claims 1-30, wherein the vector comprises a nucleotide sequence encoding an antisense oligonucleotide that reduces expression of a target gene.

32. The method of any one of claim 31, wherein the vector is selected from the group consisting of a lentivirus, retrovirus, plasmid, or herpes simplex virus (HSV).

33. The method of any one of claim 1-3 or 5-31, wherein the vector is an adeno-associated viral (AAV) vector.

34. The method of claim 33, wherein the AAV is a single-stranded AAV.

35. The method of claim 33, wherein the AAV is a self-complementary AAV.

36. The method of any one of claims 33-35, wherein the adeno-associated viral vector is any one of AAV1, scAAV1, AAV2, AAV3, AAV4, AAV5, scAAV5, AAV6, AAV7, AAV8, AAV9, scAAV9, AAV10, AAV11, AAV12, rh10, avian AAV, bovine AAV, canine AAV, equine AAV, primate AAV, non-primate AAV, or ovine AAV, or any hybrids thereof.

37. The method of any one of claims 33-36, wherein the AAV vector is AAV5.

38. The method of any one of claims 33-36, wherein the AAV vector is AAV9.

39. The method of any one of claims 33-38, wherein the vector comprises a 5′ AAV inverted terminal repeat (ITR) sequence and a 3′ AAV ITR sequence.

40. The method of any one of claims 1-39, wherein the vector is administered in a pharmaceutically acceptable carrier.

41. The method of any one of claims 1-40, wherein the vector is administered in combination with a contrast agent.

42. The method of any one of claims 1-40, wherein the vector is not administered in combination with a contrast agent.

43. The method of any one of claims 1-42, wherein the administration is by route of injection.

44. The method of any one of claims 1-43, wherein the administration is by route of infusion.

45. A method for expressing a gene of interest or a biologically active variant and/or fragment thereof comprising administering to a primate a therapeutically effective amount of an adeno-associated virus 1 (AAV1) vector or an adeno-associated virus 5 (AAV5) vector encoding the gene of interest, wherein the route of administration is selected from the group consisting of intravenous administration, intrathecal administration, intracerebroventricular administration, intraparenchymal administration, or combinations thereof.

46. The method of claim 45, wherein the primate is a human.

47. The method of claim 45, wherein the primate is a non-human primate.

48. The method of claim 47, wherein the non-human primate is an old world monkey, an orangutan, a gorilla, a chimpanzee, a crab-eating macaque, a rhesus macaque or a pig-tailed macaque.

49. The method of any one of claims 45-48, wherein the AAV1 vector or AAV5 vector comprises a nucleotide sequence operably linked to a regulatory element.

50. The method of claim 49, wherein the regulatory element is cell-type selective.

51. The method of claim 50, wherein the regulatory element is selectively expressed in a neuronal cell.

52. The method of claim 51, wherein the neuronal cells are selected from the group consisting of unipolar, bipolar, multipolar, or pseudounipolar neurons.

53. The method of claim 51, wherein the neuronal cells are GABAergic neurons.

54. The method of claim 50, wherein the regulatory element is selectively expressed in glial cells.

55. The method of claim 54, wherein the glial cells are selected from the group consisting of astrocytes, oligodendrocytes, ependymal cells, Schwann cells, and satellite cells.

56. The method of claim 49, wherein the regulatory element is selectively expressed in non-neuronal cells.

57. The method of any one of claims 45-56, wherein the AAV1 or AAV5 is administered to more than one ventricle of the brain.

58. The method of any one of claims claim 45-57, wherein the AAV1 or AAV5 is administered bilaterally.

59. The method of claim 57 or 58, wherein the AAV1 or AAV5 is administered simultaneously.

60. The method of claim 57 or 58, wherein the AAV1 or AAV5 is administered sequentially.

61. The method of claim 60, wherein each dose of the AAV1 or AAV5 is administered at least 24 hours apart.

62. The method of any one of claims 45-56, wherein the AAV1 or AAV5 is administered to one ventricle of the brain.

63. The method of any one of claims 45-62, wherein the AAV1 or AAV5 comprises a nucleotide sequence encoding a polypeptide.

64. The method of claim 63, wherein the polypeptide is a DNA binding protein.

65. The method of claim 64, wherein the DNA binding protein is selected from the group consisting of a zinc finger protein (ZFP), a zinc finger nuclease (ZFN), or a transcription activator-like effector nuclease (TALEN).

66. The method of any one of claims 63-65, wherein the nucleotide sequence is a codon-optimized variant and/or a fragment thereof.

67. The method of any one of claims 45-66, wherein the vector comprises a nucleotide sequence encoding a guide RNA (gRNA).

68. The method of any one of claims 45-68, wherein the AAV1 or AAV5 comprises a nucleotide sequence encoding an interfering RNA (RNAi) that reduces expression of a target gene.

69. The method of claim 68, wherein the RNAi reduces expression of a target gene selected from the group consisting of SOD1, HTT, Tau, or alpha-synuclein.

70. The method of any one of claims 45-69, wherein the AAV1 or AAV5 comprises a nucleotide sequence encoding an antisense oligonucleotide that reduces expression of a target gene.

71. The method of any one of claims 45-70, wherein the vector is selected from the group consisting of a lentivirus, retrovirus, plasmid, or herpes simplex virus (HSV).

72. The method of any one of claims 45-71, wherein the AAV1 or AAV5 is administered in a pharmaceutically acceptable carrier.

73. The method of any one of claims 45-72, wherein the vector is administered in combination with a contrast agent.

74. The method of any one of claims 45-72, wherein the vector is not administered in combination with a contrast agent.

75. The method of any one of claims 45-74, wherein the administration is by route of injection.

76. The method of any one of claims 45-74, wherein the administration is by route of infusion.

77. A method to inhibit or treat one or more symptoms associated with a neuronal disease in a primate in need thereof, comprising administering an adeno-associated vector (AAV) selected from the group consisting of adeno-associated vector 1 (AAV1) or adeno-associated vector 5 (AAV5) to the primate, wherein the route of administration is selected from the group consisting of intravenous administration, intrathecal administration, intracerebroventricular administration, intraparenchymal administration, or combinations thereof.

78. The method of claim 77, wherein the neuronal disease is selected from the group consisting of a lysosomal storage disease, Dravet syndrome, Alzheimer's disease, Parkinson's disease, Huntington's disease, amyotrophic lateral sclerosis (ALS), spinal muscular atrophy (SMA), epilepsy, neurodegeneration, motor disorders, movement disorders, or mood disorders.

79. The method of claim 77 or 78, wherein the primate is a human.

80. The method of claim 77 or 78, wherein the primate is a non-human primate.

81. The method of claim 80, wherein the non-human primate is an old world monkey, an orangutan, a gorilla, a chimpanzee, a crab-eating macaque, a rhesus macaque or a pig-tailed macaque.

82. A method of administering a vector to a primate, comprising intracerebroventricular (ICV) administration of a vector to the primate, wherein the vector comprises a transgene, and wherein ICV administration results in increased transgene expression in the central nervous system (CNS).

83. A method of administering a vector to a primate, comprising intracerebroventricular (ICV) administration of a vector to the primate, wherein the vector comprises a transgene, and wherein ICV administration results in increased transgene expression in the central nervous system (CNS) by at least 1.25-fold as compared to expression of the transgene when the vector is administered by any other route of administration.

84. The method of claim 82 or 83, wherein ICV administration produces at least 1.5-fold, 1.75-fold, 2-fold, 3-fold, 5-fold, 10-fold, 15-fold, 20-fold, 25-fold, 30-fold, 35-fold, 40-fold, 45-fold, 50-fold, 55-fold, 60-fold, 65-fold, 70-fold, or 75-fold greater expression of the transgene sequence in the central nervous system (CNS) as compared to expression of the transgene when the vector is administered by any other route of administration.

85. The method of claim 82 or 83, wherein ICV administration produces at least 20-90 fold, 20-80 fold, 20-70 fold, 20-60 fold, 30-90 fold, 30-80 fold, 30-70 fold, 30-60 fold, 40-90 fold, 40-80 fold, 40-70 fold, 40-60 fold, 50-90 fold, 50-80 fold, 50-70 fold, 50-60 fold, 60-90 fold, 60-80 fold, 60-70 fold, 70-90 fold, 70-80 fold, 80-90 fold greater expression of the transgene sequence in the central nervous system (CNS) as compared to expression of the transgene when the vector is administered by any other route of administration.

86. The method of any one of claim 1-44 or 82-85, wherein ICV administration results in gene transfer throughout the brain.

87. The method of claim 86, wherein the gene transfer occurs in the frontal cortex, parietal cortex, temporal cortex, hippocampus, medulla, and occipital cortex.

88. The method of any one of claim 86 or 87, wherein the gene transfer is dose dependent.

89. The method of any one of claims 82-85, wherein the vector further comprises a cell-type selective regulatory element.

90. The method of claim 89, wherein the regulatory element is selectively expressed in the brain.

91. The method of claim 90, wherein the regulatory element is selectively expressed in the frontal cortex, parietal cortex, temporal cortex, hippocampus, medulla, and/or occipital cortex.

92. The method of claim 89, wherein the regulatory element is selectively expressed in the spine.

93. The method of claim 92, wherein the regulatory element is selectively expressed in the spinal cord and/or dorsal root ganglion.

94. The method of claim 89, wherein the regulatory element is selectively expressed in neuronal cells.

95. The method of claim 94, wherein the neuronal cells are selected from the group consisting of unipolar, bipolar, multipolar, or pseudounipolar neurons.

96. The method of claim 94, wherein the neuronal cells are GABAergic neurons.

97. The method of claim 89, wherein the regulatory element is selectively expressed in non-neuronal cells.

98. The method of claim 89, wherein the regulatory element is selectively expressed in glial cells.

99. The method of claim 98, wherein the glial cells are selected from the group consisting of astrocytes, oligodendrocytes, ependymal cells, Schwann cells, and satellite cells.
